SEQUENCE LISTING (6 sequences)

SEQ ID NO: 1 - DNA, 3324 bp, Danio rerio
  CDS (1)..(3324)
atgcagtttggaaaggagttagtgaggcgaaacagcttctacaaatgtgtgataaacgaggacgaaacggaggagtctgcagatgtctttgagttggctttcaaaggaaaggcttctgccatcgatcgtctgatccagaaaggtccagaacacctaagcctcagggatgagaatggagccagtctgctgcactatgcttcagctggaggaaacctggatatcattcggctcattgtttccattgtgggccctgaggtgctgaatgtgcaggatgagcagggccgaactcctctgcactgggctgtggagcaggatcaacaacagagctgcgctgtactgctggatctgggtgctgaccccaacattctcaacaatgccctgatgggccctctgcacctggctgtcaccaaacaatacaatcatctagcagaggtccttctgtcttgtgacaaaaccaatagcaatctggagggggatctgggaaacacccctgtcatgctggcctgctccaataacaactgccaagctctccagatcctgatcaagcgcggtgccaagatgtgtattcagaacaagctgggtcactatcccattcatactgttgctttcgcaggtgccaaagaggccatggagatggtattgaaaattggagaagaacttggtgtatcatctacattgcacattaattatttggataagtccaaaagcacacctcttcatctggctgtacgcggaggcaatatcgaggtgatcaaactctgcatcttgaaaggagccaaagtagaacaacatcagagtggcaaatgcacggccctccactttgcctgcagtcagggttctctggaggctgtcaaaatcatgctttcctcttacaacagaacagaagatatcgtcaacattcgggatggagccaatcggacacctttgcatagagcaacgctgtttgatcacgttgagttggctgaatatcttatttcaaagggagcagaaattgactctattgactgtaagggactgtctccgcttctgctcgcctccagctgcagcgcttggaaaacagttgcatatctgttgtccattggtgcagattttaaaatcaaagaaaaaacaggtcgcaacttcctccactttgtcattcttcaacccaaaggcctgaaaaatcttcctgagacggttttgcagagcaccgccgtgaaggagatgttgtctgatgaggatgttgaaggctgcactcctctgcactacgcctgcaagcttggcatccacgattcggtcaaaaatatgctgggtctcaatatctgcttgggccaaaagtcacgggagaagaaatcagcgcttcacttcgctgcagaatatgggcgtataaatacatgccaccggctgctggagaccctcacagactctaaaatgctgaatgattgggatgaaaagggtctgacgcccctccatcttgcttcaagggcaggacacgctcaagtagtggatctgctgcttaggaagggagcactgtttcaaagtgattacaagggctggacctgtttacatcatgcagcagctgaaggatacacacagacgatgaaaatcctgctagcagccaatgtgaaactactggatgaaaaaaatgaagatgggaacacagcccttcatatagcagcacaagctggacatgtaagtgctgttttgctgttattggacagaggagctgaaatcgctctcaacgatgctgacaactcttttttgcatgaagctgtgcggaatgagagaagagaagtagtaaacgccaccattgaacatgagcggtgtgatgagtcaatgacgtcatttaaagcgaaatcccgctgtgttgtgctggacatcatagagttccttccagaatcttttcagcaccttctggaccagtgtattacagaatctgatcatgatgccaatagtcaggattatcatatcatgtacaatttccaatggcttcaggctcccatacagttgaagaaatatgctaaaacagacaaaacaaaagcatttcaaccactggcagcattaaatgccatggtgcggtacaaccgtctggagctcctcattcaccccttgagccgaaaatacctggagatgaagtggacggcatatggaagcaaagttcatttcctaaacttggcgatatacctgcttggattgttgcccctcacatacctcatcctcaatctgagacccagccaggacttctccaaaggcaatggcacatctgtcatcatggtgcccgtatcttttagcgagcaacaatatttaatctccgtgtgtataatcatggtgatcgtcatgaatgtgtattcgatttgcaaagaggtggtacagctggctcagcagcgagtaaattacttcacagatttctcaaaccctgcggactggagtgcagctattagcgctttggtttttgttgtacccatgtgctgcagtgtggaggccacatggcaatgggaagctggagcttatgctattctgacatcatggattggctttctgctttactttcagagatttgagcggattgggatttacgtggtgatgtttaatggaatcgtacggaccctggtgtgcatcatggttctttttgtcttccttttgctggctttcggcttggctttctacgctctgatgctccacaggccggagttcagttccatcagcctagcgttagcgcaaacgtttgtgatgacagtaggagagctaaactaccaaagcacatttcttaactcctatgaagaaggccacatggctttcccagccatcacttatttagtttttgtgttttttgttcttctcatgcccattcttctcatgaacctaatgattggtttggctgtaggagacattgcagaggtacagagaaacgcagagctgaaaaggattgctatgcagatagatcttcacactgcccttgaggagaaactaccttactggttcctgaagcgagtggacaaaccctctaccgttgtctatcccaataaatgcaagaaagtgcttctggaggccttggtgcatggtgagattcagaacacagttcgaactcgtctaaacctttgctcacggaaggaagggcttttggagagagagctgcacaagcagaagaacaggttgaaagaaatgtcgtgcatgctggaaaaacagcataacctgttgaagctgattatccagaagatggaaattacatctgaagctgatgaatatgatggtccacagaatcatggagctctaaaacagcacacctcatccacctcacagaaatcaaagtgggtcccgttactgcaagcaatcaaggccaagaagtga

SEQ ID NO: 2 - protein, 1107 aa, Danio rerio
MQFGKELVRRNSFYKCVINEDETEESADVFELAFKGKASAIDRLIQKGPEHLSLRDENGASLLHYASAGGNLDIIRLIVSIVGPEVLNVQDEQGRTPLHWAVEQDQQQSCAVLLDLGADPNILNNALMGPLHLAVTKQYNHLAEVLLSCDKTNSNLEGDLGNTPVMLACSNNNCQALQILIKRGAKMCIQNKLGHYPIHTVAFAGAKEAMEMVLKIGEELGVSSTLHINYLDKSKSTPLHLAVRGGNIEVIKLCILKGAKVEQHQSGKCTALHFACSQGSLEAVKIMLSSYNRTEDIVNIRDGANRTPLHRATLFDHVELAEYLISKGAEIDSIDCKGLSPLLLASSCSAWKTVAYLLSIGADFKIKEKTGRNFLHFVILQPKGLKNLPETVLQSTAVKEMLSDEDVEGCTPLHYACKLGIHDSVKNMLGLNICLGQKSREKKSALHFAAEYGRINTCHRLLETLTDSKMLNDWDEKGLTPLHLASRAGHAQVVDLLLRKGALFQSDYKGWTCLHHAAAEGYTQTMKILLAANVKLLDEKNEDGNTALHIAAQAGHVSAVLLLLDRGAEIALNDADNSFLHEAVRNERREVVNATIEHERCDESMTSFKAKSRCVVLDIIEFLPESFQHLLDQCITESDHDANSQDYHIMYNFQWLQAPIQLKKYAKTDKTKAFQPLAALNAMVRYNRLELLIHPLSRKYLEMKWTAYGSKVHFLNLAIYLLGLLPLTYLILNLRPSQDFSKGNGTSVIMVPVSFSEQQYLISVCIIMVIVMNVYSICKEVVQLAQQRVNYFTDFSNPADWSAAISALVFVVPMCCSVEATWQWEAGAYAILTSWIGFLLYFQRFERIGIYVVMFNGIVRTLVCIMVLFVFLLLAFGLAFYALMLHRPEFSSISLALAQTFVMTVGELNYQSTFLNSYEEGHMAFPAITYLVFVFFVLLMPILLMNLMIGLAVGDIAEVQRNAELKRIAMQIDLHTALEEKLPYWFLKRVDKPSTVVYPNKCKKVLLEALVHGEIQNTVRTRLNLCSRKEGLLERELHKQKNRLKEMSCMLEKQHNLLKLIIQKMEITSEADEYDGPQNHGALKQHTSSTSQKSKWVPLLQAIKAKK

SEQ ID NO: 3 - DNA, 7689 bp, Artificial Sequence
  Synthetic
  CDS (1480)..(4893): Trpa1b-FLAG
gtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatatacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgattacgccaagctcgaaattaaccctcactaaagggaacaaaagctggagctccaccgcggtggcggccgctctagcccgggcggatcccccgggctgcaggaattcgatatcaagcttatcgataccgtcgacctcgaggccaccatgcagtttggaaaggagttagtgaggcgaaacagcttctacaaatgtgtgataaacgaggacgaaacggaggagtctgcagatgtctttgagttggctttcaaaggaaaggcttctgccatcgatcgtctgatccagaaaggtccagaacacctaagcctcagggatgagaatggagccagtctgctgcactatgcttcagctggaggaaacctggatatcattcggctcattgtttccattgtgggccctgaggtgctgaatgtgcaggatgagcagggccgaactcctctgcactgggctgtggagcaggatcaacaacagagctgcgctgtactgctggatctgggtgctgaccccaacattctcaacaatgccctgatgggccctctgcacctggctgtcaccaaacaatacaatcatctagcagaggtccttctgtcttgtgacaaaaccaatagcaatctggagggggatctgggaaacacccctgtcatgctggcctgctccaataacaactgccaagctctccagatcctgatcaagcgcggtgccaagatgtgtattcagaacaagctgggtcactatcccattcatactgttgctttcgcaggtgccaaagaggccatggagatggtattgaaaattggagaagaacttggtgtatcatctacattgcacattaattatttggataagtccaaaagcacacctcttcatctggctgtacgcggaggcaatatcgaggtgatcaaactctgcatcttgaaaggagccaaagtagaacaacatcagagtggcaaatgcacggccctccactttgcctgcagtcagggttctctggaggctgtcaaaatcatgctttcctcttacaacagaacagaagatatcgtcaacattcgggatggagccaatcggacacctttgcatagagcaacgctgtttgatcacgttgagttggctgaatatcttatttcaaagggagcagaaattgactctattgactgtaagggactgtctccgcttctgctcgcctccagctgcagcgcttggaaaacagttgcatatctgttgtccattggtgcagattttaaaatcaaagaaaaaacaggtcgcaacttcctccactttgtcattcttcaacccaaaggcctgaaaaatcttcctgagacggttttgcagagcaccgccgtgaaggagatgttgtctgatgaggatgttgaaggctgcactcctctgcactacgcctgcaagcttggcatccacgattcggtcaaaaatatgctgggtctcaatatctgcttgggccaaaagtcacgggagaagaaatcagcgcttcacttcgctgcagaatatgggcgtataaatacatgccaccggctgctggagaccctcacagactctaaaatgctgaatgattgggatgaaaagggtctgacgcccctccatcttgcttcaagggcaggacacgctcaagtagtggatctgctgcttaggaagggagcactgtttcaaagtgattacaagggctggacctgtttacatcatgcagcagctgaaggatacacacagacgatgaaaatcctgctagcagccaatgtgaaactactggatgaaaaaaatgaagatgggaacacagcccttcatatagcagcacaagctggacatgtaagtgctgttttgctgttattggacagaggagctgaaatcgctctcaacgatgctgacaactcttttttgcatgaagctgtgcggaatgagagaagagaagtagtaaacgccaccattgaacatgagcggtgtgatgagtcaatgacgtcatttaaagcgaaatcccgctgtgttgtgctggacatcatagagttccttccagaatcttttcagcaccttctggaccagtgtattacagaatctgatcatgatgccaatagtcaggattatcatatcatgtacaatttccaatggcttcaggctcccatacagttgaagaaatatgctaaaacagacaaaacaaaagcatttcaaccactggcagcattaaatgccatggtgcggtacaaccgtctggagctcctcattcaccccttgagccgaaaatacctggagatgaagtggacggcatatggaagcaaagttcatttcctaaacttggcgatatacctgcttggattgttgcccctcacatacctcatcctcaatctgagacccagccaggacttctccaaaggcaatggcacatctgtcatcatggtgcccgtatcttttagcgagcaacaatatttaatctccgtgtgtataatcatggtgatcgtcatgaatgtgtattcgatttgcaaagaggtggtacagctggctcagcagcgagtaaattacttcacagatttctcaaaccctgcggactggagtgcagctattagcgctttggtttttgttgtacccatgtgctgcagtgtggaggccacatggcaatgggaagctggagcttatgctattctgacatcatggattggctttctgctttactttcagagatttgagcggattgggatttacgtggtgatgtttaatggaatcgtacggaccctggtgtgcatcatggttctttttgtcttccttttgctggctttcggcttggctttctacgctctgatgctccacaggccggagttcagttccatcagcctagcgttagcgcaaacgtttgtgatgacagtaggagagctaaactaccaaagcacatttcttaactcctatgaagaaggccacatggctttcccagccatcacttatttagtttttgtgttttttgttcttctcatgcccattcttctcatgaacctaatgattggtttggctgtaggagacattgcagaggtacagagaaacgcagagctgaaaaggattgctatgcagatagatcttcacactgcccttgaggagaaactaccttactggttcctgaagcgagtggacaaaccctctaccgttgtctatcccaataaatgcaagaaagtgcttctggaggccttggtgcatggtgagattcagaacacagttcgaactcgtctaaacctttgctcacggaaggaagggcttttggagagagagctgcacaagcagaagaacaggttgaaagaaatgtcgtgcatgctggaaaaacagcataacctgttgaagctgattatccagaagatggaaattacatctgaagctgatgaatatgatggtccacagaatcatggagctctaaaacagcacacctcatccacctcacagaaatcaaagtgggtcccgttactgcaagcaatcaaggccaagaagtcaggttggaccggtgccaccatggactataaggaccacgacggagactacaaggatcatgatattgattacaaagacgatgacgataagtagggtaccaggtaagtgtacccaattcgccctatagtgagtcgtattacaattcactcgatcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagaatcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagaacatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagaacttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagc

SEQ ID NO: 4 - protein, 1137 aa, Artificial Sequence
  Synthetic Construct
MQFGKELVRRNSFYKCVINEDETEESADVFELAFKGKASAIDRLIQKGPEHLSLRDENGASLLHYASAGGNLDIIRLIVSIVGPEVLNVQDEQGRTPLHWAVEQDQQQSCAVLLDLGADPNILNNALMGPLHLAVTKQYNHLAEVLLSCDKTNSNLEGDLGNTPVMLACSNNNCQALQILIKRGAKMCIQNKLGHYPIHTVAFAGAKEAMEMVLKIGEELGVSSTLHINYLDKSKSTPLHLAVRGGNIEVIKLCILKGAKVEQHQSGKCTALHFACSQGSLEAVKIMLSSYNRTEDIVNIRDGANRTPLHRATLFDHVELAEYLISKGAEIDSIDCKGLSPLLLASSCSAWKTVAYLLSIGADFKIKEKTGRNFLHFVILQPKGLKNLPETVLQSTAVKEMLSDEDVEGCTPLHYACKLGIHDSVKNMLGLNICLGQKSREKKSALHFAAEYGRINTCHRLLETLTDSKMLNDWDEKGLTPLHLASRAGHAQVVDLLLRKGALFQSDYKGWTCLHHAAAEGYTQTMKILLAANVKLLDEKNEDGNTALHIAAQAGHVSAVLLLLDRGAEIALNDADNSFLHEAVRNERREVVNATIEHERCDESMTSFKAKSRCVVLDIIEFLPESFQHLLDQCITESDHDANSQDYHIMYNFQWLQAPIQLKKYAKTDKTKAFQPLAALNAMVRYNRLELLIHPLSRKYLEMKWTAYGSKVHFLNLAIYLLGLLPLTYLILNLRPSQDFSKGNGTSVIMVPVSFSEQQYLISVCIIMVIVMNVYSICKEVVQLAQQRVNYFTDFSNPADWSAAISALVFVVPMCCSVEATWQWEAGAYAILTSWIGFLLYFQRFERIGIYVVMFNGIVRTLVCIMVLFVFLLLAFGLAFYALMLHRPEFSSISLALAQTFVMTVGELNYQSTFLNSYEEGHMAFPAITYLVFVFFVLLMPILLMNLMIGLAVGDIAEVQRNAELKRIAMQIDLHTALEEKLPYWFLKRVDKPSTVVYPNKCKKVLLEALVHGEIQNTVRTRLNLCSRKEGLLERELHKQKNRLKEMSCMLEKQHNLLKLIIQKMEITSEADEYDGPQNHGALKQHTSSTSQKSKWVPLLQAIKAKKSGWTGATMDYKDHDGDYKDHDIDYKDDDDK

SEQ ID NO: 5 - DNA, 7112 bp, Artificial Sequence
  Synthetic
  CDS (780)..(4877): Trpa1b-2A-mCherry
catagaaaaatatttataacatatataacatttagaattctgcaacaaaccaaaacaatttggccttaaataatatttgacagatatgatgtgatataacaaaatacattagaaaaatgtgcaaaatgtgtcttgcagcctcaggccacttttgctgaagtccattcttttcgatttcctcttgagatttaagttaaagtgggcttgttgcatgcaggacagacagcaacagctccgctcgcgtggccggacagttctcctgctctctgattggccagcggtgattacaggcacgcttcagagggcgaacaaacaacaggcataaattatgctaatgagatggtcgtgcggcacggggcagacctgatgcaaaccattcattcatccttcacaaaaatgccaaagcgaggggggtatgtccattgtcccatcagggtatataaggtaggtgaggccagtcgtgtgccaagctcacaagctcacacgagctgattgagggttaagagcaagacagacgcgtgccattatcttcaacacaaactatctgcaggattctgcaaaacctcaagcatctcccagcccaccaataaggtgcgtattgggtcacgcagataacttgaaaggactttgcacgactgatgttgttttttgtttcttttctggcgtattcctaacctgattttgtaaatttctccaggttatcaaccggtggagctccagcttttgttccctttagtgagggttaattcaagtttgtacaaaaaagcggcttcgccaccatgcagtttggaaaggagttagtgaggcgaaacagcttctacaaatgtgtgataaacgaggacgaaacggaggagtctgcagatgtctttgagttggctttcaaaggaaaggcttctgccatcgatcgtctgatccagaaaggtccagaacacctaagcctcagggatgagaatggagccagtctgctgcactatgcttcagctggaggaaacctggatatcattcggctcattgtttccattgtgggccctgaggtgctgaatgtgcaggatgagcagggccgaactcctctgcactgggctgtggagcaggatcaacaacagagctgcgctgtactgctggatctgggtgctgaccccaacattctcaacaatgccctgatgggccctctgcacctggctgtcaccaaacaatacaatcatctagcagaggtccttctgtcttgtgacaaaaccaatagcaatctggagggggatctgggaaacacccctgtcatgctggcctgctccaataacaactgccaagctctccagatcctgatcaagcgcggtgccaagatgtgtattcagaacaagctgggtcactatcccattcatactgttgctttcgcaggtgccaaagaggccatggagatggtattgaaaattggagaagaacttggtgtatcatctacattgcacattaattatttggataagtccaaaagcacacctcttcatctggctgtacgcggaggcaatatcgaggtgatcaaactctgcatcttgaaaggagccaaagtagaacaacatcagagtggcaaatgcacggccctccactttgcctgcagtcagggttctctggaggctgtcaaaatcatgctttcctcttacaacagaacagaagatatcgtcaacattcgggatggagccaatcggacacctttgcatagagcaacgctgtttgatcacgttgagttggctgaatatcttatttcaaagggagcagaaattgactctattgactgtaagggactgtctccgcttctgctcgcctccagctgcagcgcttggaaaacagttgcatatctgttgtccattggtgcagattttaaaatcaaagaaaaaacaggtcgcaacttcctccactttgtcattcttcaacccaaaggcctgaaaaatcttcctgagacggttttgcagagcaccgccgtgaaggagatgttgtctgatgaggatgttgaaggctgcactcctctgcactacgcctgcaagcttggcatccacgattcggtcaaaaatatgctgggtctcaatatctgcttgggccaaaagtcacgggagaagaaatcagcgcttcacttcgctgcagaatatgggcgtataaatacatgccaccggctgctggagaccctcacagactctaaaatgctgaatgattgggatgaaaagggtctgacgcccctccatcttgcttcaagggcaggacacgctcaagtagtggatctgctgcttaggaagggagcactgtttcaaagtgattacaagggctggacctgtttacatcatgcagcagctgaaggatacacacagacgatgaaaatcctgctagcagccaatgtgaaactactggatgaaaaaaatgaagatgggaacacagcccttcatatagcagcacaagctggacatgtaagtgctgttttgctgttattggacagaggagctgaaatcgctctcaacgatgctgacaactcttttttgcatgaagctgtgcggaatgagagaagagaagtagtaaacgccaccattgaacatgagcggtgtgatgagtcaatgacgtcatttaaagcgaaatcccgctgtgttgtgctggacatcatagagttccttccagaatcttttcagcaccttctggaccagtgtattacagaatctgatcatgatgccaatagtcaggattatcatatcatgtacaatttccaatggcttcaggctcccatacagttgaagaaatatgctaaaacagacaaaacaaaagcatttcaaccactggcagcattaaatgccatggtgcggtacaaccgtctggagctcctcattcaccccttgagccgaaaatacctggagatgaagtggacggcatatggaagcaaagttcatttcctaaacttggcgatatacctgcttggattgttgcccctcacatacctcatcctcaatctgagacccagccaggacttctccaaaggcaatggcacatctgtcatcatggtgcccgtatcttttagcgagcaacaatatttaatctccgtgtgtataatcatggtgatcgtcatgaatgtgtattcgatttgcaaagaggtggtacagctggctcagcagcgagtaaattacttcacagatttctcaaaccctgcggactggagtgcagctattagcgctttggtttttgttgtacccatgtgctgcagtgtggaggccacatggcaatgggaagctggagcttatgctattctgacatcatggattggctttctgctttactttcagagatttgagcggattgggatttacgtggtgatgtttaatggaatcgtacggaccctggtgtgcatcatggttctttttgtcttccttttgctggctttcggcttggctttctacgctctgatgctccacaggccggagttcagttccatcagcctagcgttagcgcaaacgtttgtgatgacagtaggagagctaaactaccaaagcacatttcttaactcctatgaagaaggccacatggctttcccagccatcacttatttagtttttgtgttttttgttcttctcatgcccattcttctcatgaacctaatgattggtttggctgtaggagacattgcagaggtacagagaaacgcagagctgaaaaggattgctatgcagatagatcttcacactgcccttgaggagaaactaccttactggttcctgaagcgagtggacaaaccctctaccgttgtctatcccaataaatgcaagaaagtgcttctggaggccttggtgcatggtgagattcagaacacagttcgaactcgtctaaacctttgctcacggaaggaagggcttttggagagagagctgcacaagcagaagaacaggttgaaagaaatgtcgtgcatgctggaaaaacagcataacctgttgaagctgattatccagaagatggaaattacatctgaagctgatgaatatgatggtccacagaatcatggagctctaaaacagcacacctcatccacctcacagaaatcaaagtgggtcccgttactgcaagcaatcaaggccaagaagggatccggagccacgaacttctctctgttaaagcaagcaggagacgtggaagaaaaccccggtcctatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaagacccagctttcttgtacaaagtgggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttccaactttattatacatagttgataattcactggccgtcgttttacggtaccatcgatgatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagatcagatctgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctggttcttgaccccctaccttcagcaagcccagcagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagg

SEQ ID NO: 6 - protein, 1365 aa, Artificial Sequence
  Synthetic Construct
MQFGKELVRRNSFYKCVINEDETEESADVFELAFKGKASAIDRLIQKGPEHLSLRDENGASLLHYASAGGNLDIIRLIVSIVGPEVLNVQDEQGRTPLHWAVEQDQQQSCAVLLDLGADPNILNNALMGPLHLAVTKQYNHLAEVLLSCDKTNSNLEGDLGNTPVMLACSNNNCQALQILIKRGAKMCIQNKLGHYPIHTVAFAGAKEAMEMVLKIGEELGVSSTLHINYLDKSKSTPLHLAVRGGNIEVIKLCILKGAKVEQHQSGKCTALHFACSQGSLEAVKIMLSSYNRTEDIVNIRDGANRTPLHRATLFDHVELAEYLISKGAEIDSIDCKGLSPLLLASSCSAWKTVAYLLSIGADFKIKEKTGRNFLHFVILQPKGLKNLPETVLQSTAVKEMLSDEDVEGCTPLHYACKLGIHDSVKNMLGLNICLGQKSREKKSALHFAAEYGRINTCHRLLETLTDSKMLNDWDEKGLTPLHLASRAGHAQVVDLLLRKGALFQSDYKGWTCLHHAAAEGYTQTMKILLAANVKLLDEKNEDGNTALHIAAQAGHVSAVLLLLDRGAEIALNDADNSFLHEAVRNERREVVNATIEHERCDESMTSFKAKSRCVVLDIIEFLPESFQHLLDQCITESDHDANSQDYHIMYNFQWLQAPIQLKKYAKTDKTKAFQPLAALNAMVRYNRLELLIHPLSRKYLEMKWTAYGSKVHFLNLAIYLLGLLPLTYLILNLRPSQDFSKGNGTSVIMVPVSFSEQQYLISVCIIMVIVMNVYSICKEVVQLAQQRVNYFTDFSNPADWSAAISALVFVVPMCCSVEATWQWEAGAYAILTSWIGFLLYFQRFERIGIYVVMFNGIVRTLVCIMVLFVFLLLAFGLAFYALMLHRPEFSSISLALAQTFVMTVGELNYQSTFLNSYEEGHMAFPAITYLVFVFFVLLMPILLMNLMIGLAVGDIAEVQRNAELKRIAMQIDLHTALEEKLPYWFLKRVDKPSTVVYPNKCKKVLLEALVHGEIQNTVRTRLNLCSRKEGLLERELHKQKNRLKEMSCMLEKQHNLLKLIIQKMEITSEADEYDGPQNHGALKQHTSSTSQKSKWVPLLQAIKAKKGSGATNFSLLKQAGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

(19) United States
(12) Patent Application Publication
(10) Pub. No.: US 2022/0280644 A1
(43) Pub. Date: Sep. 8, 2022

(54) "TRPswitch" - A STEP FUNCTION CHEMO-OPTOGENETIC LIGAND

(71) Applicants: UNIVERSITY OF UTAH RESEARCH FOUNDATION, Salt Lake City, UT (US); IMPERIAL COLLEGE INNOVATIONS LIMITED, London (GB)

(72) Inventors: Randall Theodore Peterson, Salt Lake City, UT (US); Pui Ying Lam, Salt Lake City, UT (US); Matthew J. Fuchter, London (GB); Aditya Raymond Thawani, London (GB)

(21) Appl. No.: 17/632,332

(22) PCT Filed: Aug. 7, 2020
(86) PCT No.: PCT/US2020/045317

§ 371 Date: Feb. 2, 2022

Related U.S. Application Data
(60) Provisional application No. 62/885,102, filed on Aug. 9, 2019.

(51) Int. Cl. A61K 41/00; C07D 307/68; C07D 333/38; C07D 405/14; C07D 409/14

(52) U.S. Cl. CPC A61K 41/0057; C07D 307/68; C07D 333/38; C07D 405/14; C07D 409/14

(57) ABSTRACT

Described herein are photoswitchable compounds that can activate TRPA1 channels in neuronal and non-neuronal cells. The TRPswitch molecules allow for optical control of both the activation and deactivation of TRPA1 channels. Such compounds can be used as research tools or therapeutics.

### CROSS-REFERENCE TO RELATED APPLICATIONS

This application claims priority to U.S. Provisional Patent Application No. 62/885,102, filed on Aug. 9, 2019, which is incorporated by reference herein in its entirety.

### FEDERALLY SPONSORED RESEARCH

This invention was made with United States government support under National Institutes of Health grant numbers R01DK082871 and K99NS112599. The United States government has certain rights in the invention.

### REFERENCE TO SEQUENCE LISTING

This application is filed with a Computer Readable Form of a Sequence Listing in accord with 37 C.F.R. § 1.821(c). The text file submitted by EFS, “026389-9276-WO01_sequence_listing_5 Aug. 2020_ST25.txt,” was created on Aug. 5, 2020, contains 6 sequences, has a file size of 92.3 Kbytes, and is hereby incorporated by reference in its entirety.

### TECHNICAL FIELD

Described herein are photoswitchable compounds that can activate the TRPA1 channel in neuronal and non-neuronal cells. The TRPswitch molecules allow for optical control of both the activation and deactivation of the TRPA1 channel. Such compounds can be used as research tools or therapeutics.

### BACKGROUND

Optogenetics has proven to be a transformative technology for various fields of basic research, particularly in neuroscience. It allows for a non-invasive, localized, and temporally selective optical modulation of selected cells within an animal. Optogenetic technologies hold great promise for clinical applications. For example, preclinical animal models have demonstrated potential utility in treating retinitis pigmentosa with AAV-delivered channelrhodopsin2 (ChR2) [1]. However, high expression of the light-gated ion channel ChR2, originating from green algae, has cytotoxic effects [2]. It remains unclear whether or not expression of ChR2 in humans will result in immune rejection or inflammation. Therefore, to advance the utility of optogenetics in clinical applications, development of optogenetic actuators that are vertebrate in origin or endogenous to humans, as well as those based on ion channels with high unitary conductance, will be advantageous. The use of vertebrate protein actuators should reduce the risk of immunological reactions due to long-term expression of exogenous proteins. High conductance actuators should also decrease the amount of channel expression needed for sufficient light-controlled activity. Some progress has been made toward these goals, including the development of an endogenous protein-targeting photoswitch that confers light-sensitivity on endogenous neuronal ion channels, proposed to have the potential to restore vision to the blind [3-5].

Discovery of new optogenetic actuators that possess novel and unique properties will undoubtedly enhance the ability to dissect biological systems such as the complex neuronal networks of the brain. For example, optogenetic experiments that require long activation periods would benefit greatly from “step function” optogenetic tools that allow stable, bi-directional on and off switching of channels. Step function opsins (SFOs) are engineered versions of ChR2 that provide longer off-time constants [6-8]. As such, they are useful in modifying the spontaneous firing rate of neurons, as well as in applications where behavioral analysis without continuous optic fiber tethering is desired [9-10]. However, as mentioned above, the non-vertebrate origin of these opsins present potential challenges in clinical applications. As a complementary approach, chemo-optogenetic tools that combine chemicals and optogenetics have been under rapid development. Using a chemical design approach, photochromic soluble ligands (PCLs) for various wild-type ion channels have been synthesized to allow for light-controlled channel activity [11-15]. These photoswitchable PCLs block and unblock their corresponding ion channel either in their E or Z configuration. Examples include PCLs for the TRPV1 channel [11], NMDA receptor [16-17], and kainate receptor [18-19].

Transient Receptor Potential Ankyrin 1 (TRPA1) is a member of the Transient Receptor Potential (TRP) channel family. It is a non-selective cation channel that plays an important role in inflammatory and neuropathic pain, itch, and respiratory diseases [20-22]. Photoactivable ligands for TRPA1, such as optovin, have the ability to act as chemo-optogenetic tools [23-24]. TRPA1 has a channel conductance of approximately 100 pS [25], 1,000 times greater than ChR2, making it ideal for applications where high conductance or low expression levels are desired. Importantly however, while photochemical stimulation of optovin-class chemo-optogenetic ligands activate TRPA1 rapidly (in low millisecond time scales), channel deactivation depends upon spontaneous (i.e. non-photochemical) reversal of ligand action, which occurs on the time scale of seconds. In other words, the approach is somewhat akin to photodecaging, where a “protected” ligand is activated (“deprotected”) following a light stimulus [26], but where deactivation requires spontaneous and often slow dissipation of the ligand. It would be a great advancement to develop a chemo-optogenetic system that preserves the high conductance of TRPA1 and the rapid activation of optovin-class ligands, but enables rapid, light-controlled channel deactivation: a photoreversible/photoswitchable system.

What is needed are photoswitchable compounds that can bind to and activate or deactivate the TRPA1 channel in neuronal and non-neuronal cells.

### SUMMARY

One embodiment described herein is a photoactive compound of Formula (I):

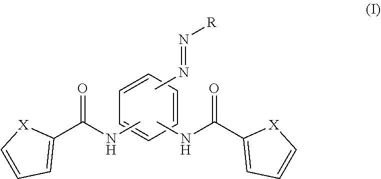

wherein each X is independently O or S and R is a substituted or unsubstituted heteroaryl moiety or a substituted phenyl moiety.

In another embodiment, the compound is one of Formulae (II), (Ill), or (IV):

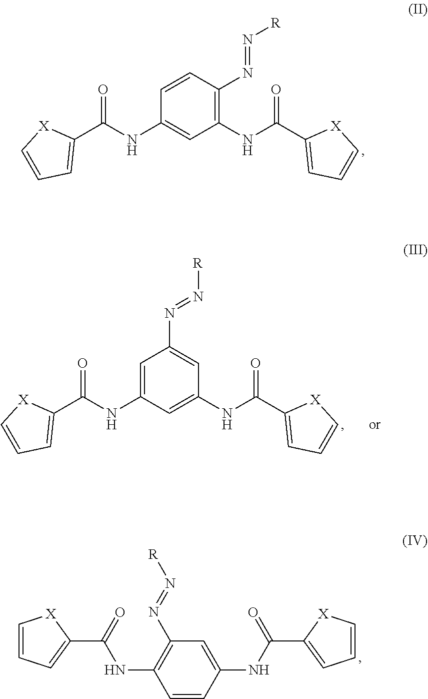

wherein each X is independently O or S and R is a substituted or unsubstituted heteroaryl moiety or a substituted phenyl moiety. In one aspect, each X is independently O or S and R is a mono- or bi-cyclic aryl ring or a 5-10 membered mono or bi-cyclic heteroaryl ring optionally substituted with one or more of Q₁-(R₁)ₙ; Q₁ is a covalent bond, H, O, halogen, cyano, —NR₃—, —CONR₂—, —NR₂CO—, oxo, nitro, —S(O)ₘ—, —C₁₋₆ haloalkyl, —C₁₋₆ alkoxy, —C₁₋₆ haloalkoxy, —C₁₋₆ hydroxyalkyl, —C₁₋₆ cyanoalkyl, —CO—, —SO₂R₃, —NR₃R₄, —NR₃COR₄, —NR₂CONR₃R₄, —CONR₃R₄, —CO₂R₃, —NR₃CO₂R₄, —SO₂NR₃R₄, —CONR₃, —C(O)R₃, —NR₃SO₂R₄, —NR₂SO₂NR₃R₄, —SO₂NR₃, optionally substituted —C₁₋₆ alkylene, optionally substituted —C₂₋₆ alkenylene, or optionally substituted —C₁₋₆ alkyl; R₁ is halogen, oxo, cyano, nitro, optionally substituted —C₁₋₆ haloalkyl, optionally substituted —C₁₋₆ alkoxy, optionally substituted —C₁₋₆ haloalkoxy, optionally substituted —C₁₋₆ alkyl, optionally substituted —C₂₋₆ alkenyl, optionally substituted —C₂₋₆ alkynyl, —C₁-C₆ hydroxyalkyl, optionally substituted heterocyclyl, optionally substituted cycloalkyl, optionally substituted heteroaryl, optionally substituted aryl, -Q₂-NR₅CONR₆R₇, -Q₂-NR₅R₆, -Q₂-NR₅COR₆, -Q₂-COR₅, -Q₂-SO₂R₅, -Q₂-CONR₅, -Q₂-CONR₅R₆, -Q₂-CO₂R₅, -Q₂-SO₂NR₅R₆, -Q₂-NR₅SO₂R₆, or -Q₂-NR₅SO₂NR₆R₇; Q₂ is a covalent bond, —C₁₋₆ alkyl, —C₁₋₆ alkylene, or —C₂₋₆ alkenylene; R₂, R₃, and R₄ are each independently hydrogen, optionally substituted —C₁₋₆ alkyl, or optionally substituted —C₁₋₆ alkylene; R₅, R₆, and R₇ are each independently H, optionally substituted —C₁₋₆ alkyl, optionally substituted heterocyclyl, optionally substituted heteroaryl, optionally substituted aryl, or optionally substituted cycloalkyl; n is 0, 1, 2, 3, or 4; when n is 1, 2, 3, or 4, R₁ is an optionally substituted 3-10 membered heterocyclyl, heteroaryl, aryl, or a mono- or bi-cycloalkyl ring; and wherein n is 0, Q is present and R₁ is absent; m is 0, 1, or 2; and any of the compounds designated as “optionally substituted” may be substituted with halogen, —C₁₋₆ alkyl, —C₁₋₆ alkenyl, —C₁₋₆ alkynyl, —C₁₋₆ alkoxy, —C₀₋₆ amine, —C₀₋₆ amide, —C₀₋₆—OH, —C₀₋₆—COOH, —C₀₋₆ CN, or C₁₋₆ halogen. In one aspect, each X is S. In another aspect, each X is O.

In another embodiment, the compound is Formula (V):

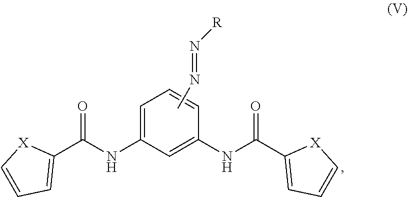

wherein each X is S or O; R is

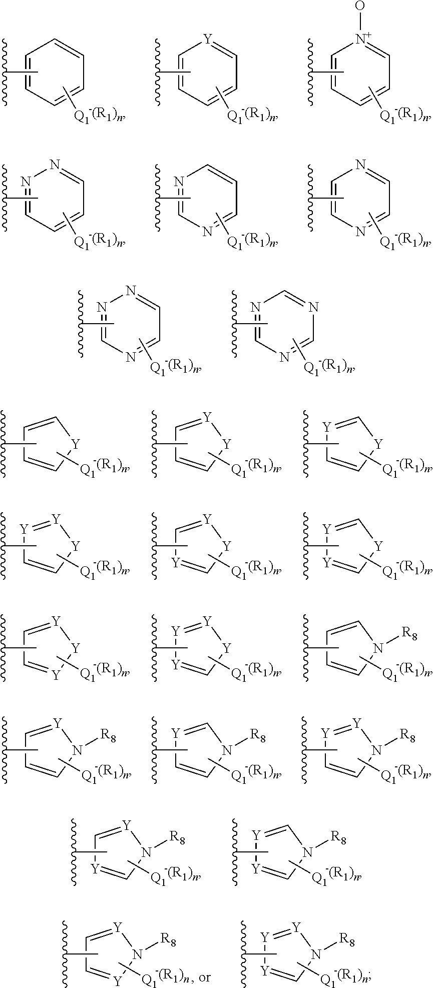

Y is independently O, S, or N; Q₁ is a covalent bond, H, O, halogen, cyano, —NR₃—, —CONR₂—, —NR₂CO—, oxo, nitro, —S(O)ₘ—, C₁₋₆ haloalkyl, C₁₋₆ alkoxy, C₁₋₆ haloalkoxy, C₁₋₆ hydroxyalkyl, C₁₋₆ cyanoalkyl, —CO—, —SO₂R₃, —NR₃R₄, —NR₃COR₄, —NR₂CONR₃R₄, —CONR₃R₄, —CO₂R₃, —NR₃CO₂R₄, —SO₂NR₃R₄, —CONR₃, —C(O)R₃, —NR₃SO₂R₄, —NR₂SO₂NR₃R₄, —SO₂NR₃, optionally substituted —C₁₋₆ alkylene, optionally substituted —C₂₋₆ alkenylene, or optionally substituted —C₁₋₆ alkyl; R₁ is halogen, oxo, cyano, nitro, optionally substituted —C₁₋₆ haloalkyl, optionally substituted —C₁₋₆ alkoxy, optionally substituted —C₁₋₆ haloalkoxy, optionally substituted —C₁₋₆ alkyl, optionally substituted —C₂₋₆ alkenyl, optionally substituted —C₂₋₆ alkynyl, —C₁-C₆ hydroxyalkyl, optionally substituted heterocyclyl, optionally substituted cycloalkyl, optionally substituted heteroaryl, optionally substituted aryl, -Q₂-NR₅CONR₆R₇, -Q₂-NR₅R₆, -Q₂-NR₅COR₆, -Q₂-COR₅, -Q₂-SO₂R₅, -Q₂-CONR₅, -Q₂-CONR₅R₆, -Q₂-CO₂R₅, -Q₂-SO₂NR₅R₆, -Q₂-NR₅SO₂R₆, or -Q₂-NR₅SO₂NR₆R₇; Q₂ is a covalent bond, —C₁₋₆ alkyl, —C₁₋₆ alkylene, or —C₂₋₆ alkenylene; R₂, R₃, and R₄ are each independently hydrogen, optionally substituted —C₁₋₆ alkyl, or optionally substituted —C₁₋₆ alkylene; R₅, R₆, and R₇ are each independently H, optionally substituted —C₁₋₆ alkyl, optionally substituted heterocyclyl, optionally substituted heteroaryl, optionally substituted aryl, or optionally substituted cycloalkyl; R₃ is a covalent bond, hydrogen, halogen, oxygen, oxo, nitro, cyano, —NR₃—, —CONR₃—, —NR₃CO—, —S(O)ₘ—, C₁-C₆ haloalkyl, —C₁-C₆ alkoxy, —C₁-C₆ haloalkoxy, —C₁-C₆ hydroxyalkyl, —C₁-C₆ cyanoalkyl, —CO—, —SO₂R₄, —NR₃R₄, —NR₃COR₄, —NR₂CONR₃R₄, —CONR₃R₄, —CO₂R₃, —NR₃CO₂R₄, —SO₂NR₃R₄, —CONR₃, —C(O)R₃, —NR₃SO₂R₄, —NR₂SO₂NR₃R₄, —SO₂NR₃, optionally substituted C₁-C₆ alkylene, optionally substituted —C₂-C₆ alkenylene, or optionally substituted —C₁-C₆ alkyl; n is 0, 1, 2, 3, or 4; when n is 1, 2, 3, or 4, R₁ is an optionally substituted 3-10 membered heterocyclyl, heteroaryl, aryl, or a mono- or bi-cycloalkyl ring; and wherein n is 0, Q is present and R₁ is absent; m is 0, 1, or 2; and any of the compounds designated as “optionally substituted” may be substituted with halogen, —C₀₋₆ alkyl, —C₁₋₆ alkenyl, —C₁₋₆ alkynyl, —C₁₋₆ alkoxy, —C₀₋₆ amine, —C₀₋₆ amide, —C₀₋₆—OH, —C₀₋₆—COOH, —C₀₋₆ CN, or C₁₋₆ halogen.
In one aspect, each X is S or O and R is:

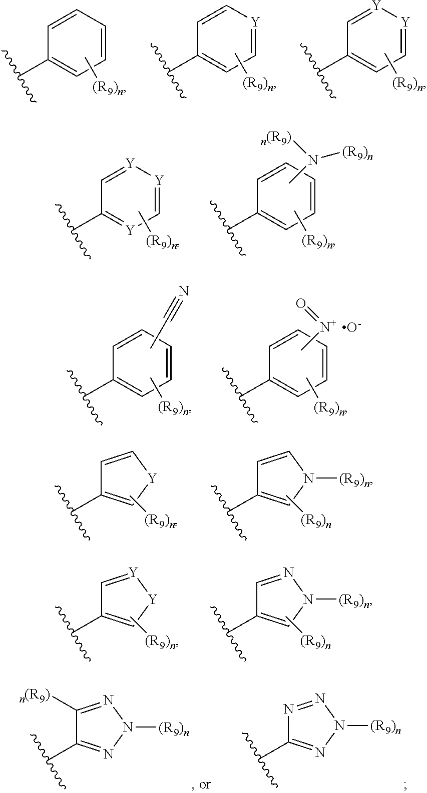

Y is independently O, S, or N; R₉ is independently H, halogen, C₁₋₆ alkyl, C₁₋₆ alkenyl, C₁₋₆ alkynyl, C₁₋₆ alkoxy, C₀₋₆ amine, C₀₋₆ amide, C₀₋₆—OH, C₀₋₆—COOH, C₀₋₆ CN, C₁₋₆ halogen, or —CF₃; and n is 0, 1, 2, 3, or 4. In another aspect, each X is S or O and R is:

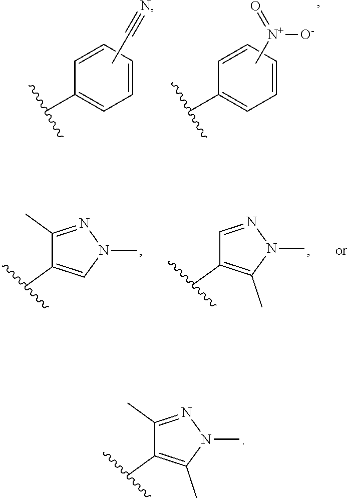

In another aspect, each X is S or O and R is:

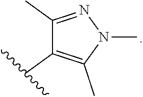 [SMILES: *c1c(C)nn(C)c1C]

In another embodiment, the compound is selected from:

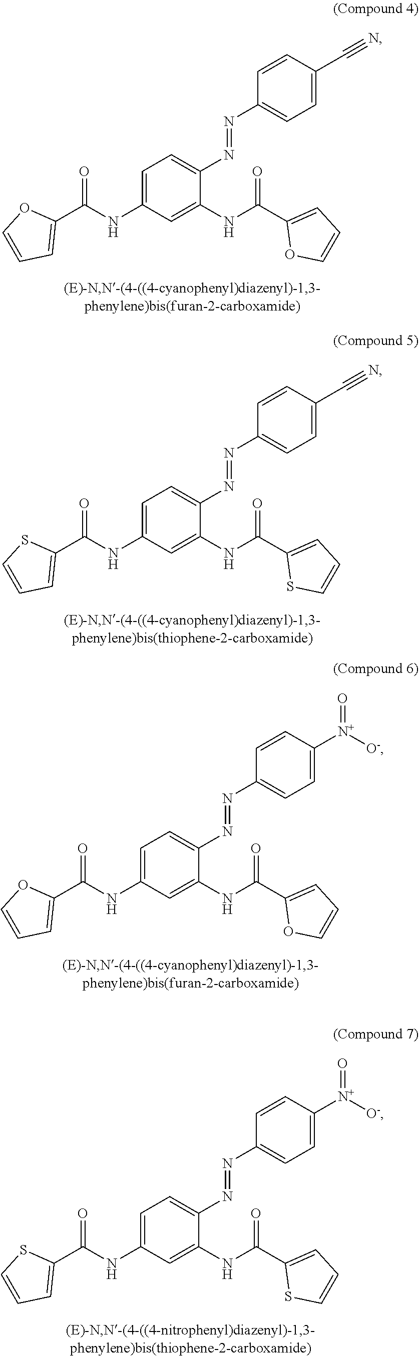

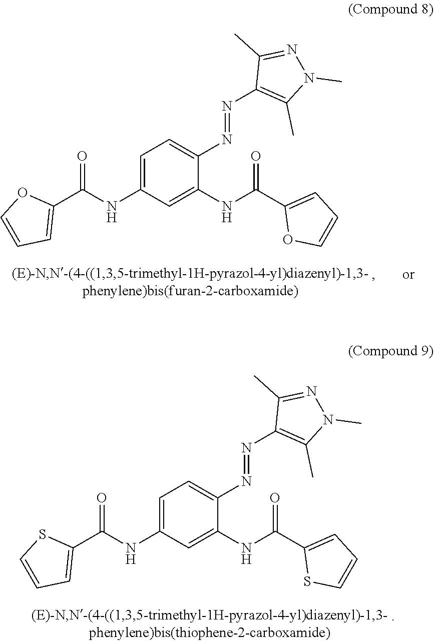 [SMILES: Cc1nn(C)c(C)c1/N=N/c1ccc(NC(=O)c2ccco2)cc1NC(=O)c1ccco1 ; Cc1nn(C)c(C)c1/N=N/c1ccc(NC(=O)c2cccs2)cc1NC(=O)c1cccs1]

In another aspect, each X is S or O, and R is a substituted or unsubstituted arylazopyrazole.
In another aspect, each X is S or O and R is

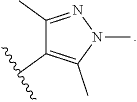

In another embodiment, the compound is:

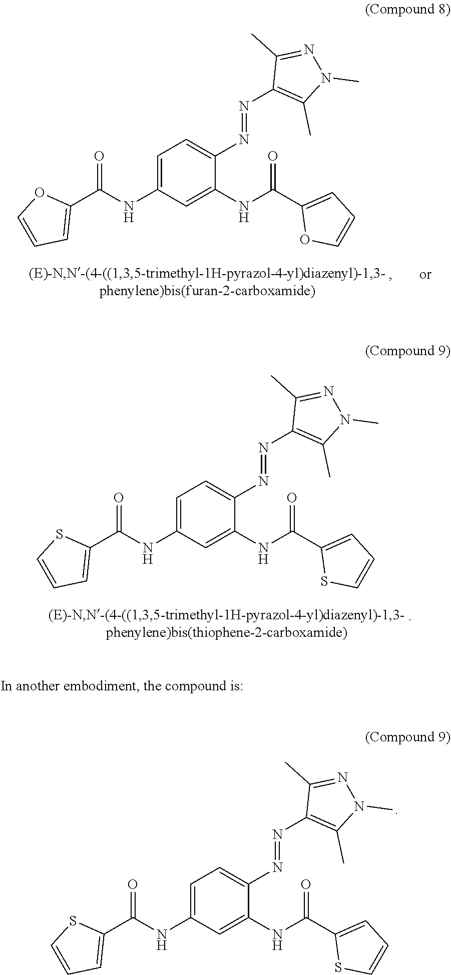 [SMILES: Cc1nn(C)c(C)c1/N=N/c1ccc(NC(=O)c2ccco2)cc1NC(=O)c1ccco1 ; Cc1nn(C)c(C)c1/N=N/c1ccc(NC(=O)c2cccs2)cc1NC(=O)c1cccs1 ; Cc1nn(C)c(C)c1/N=N/c1ccc(NC(=O)c2cccs2)cc1NC(=O)c1cccs1]

Another embodiment described herein is a compound is a reversible photoswitch that acts on a TRPA1 channel.

Another embodiment described herein is a research tool comprising any of the compounds described herein.

Another embodiment described herein is a method for reversibly activating or deactivating a TRPA1 channel, the method comprising: contacting a TRPA1 channel with an E isomer of any of the compounds described herein; pulse illuminating the compound with violet light (˜350-405 nm) to induce an E→Z isomerization and activate the TRPA1 channel; and subsequently, pulse illuminating the compound with green light (˜500-600 nm) to induce a Z→E isomerization and deactivate the TRPA1 channel. In one aspect, the compound is:

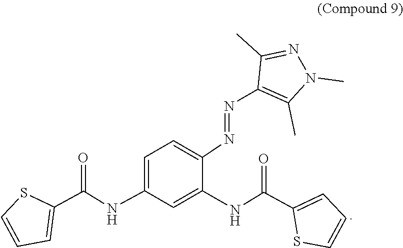 [SMILES: Cc1nn(C)c(C)c1/N=N/c1ccc(NC(=O)c2cccs2)cc1NC(=O)c1cccs1]

In another aspect, the compound has a concentration of about 10-20 μM. In another aspect, the compound is administered to an organism, part thereof, or cell culture, and the organism, part thereof, or cell culture is pulse illuminated with violet light to activate and subsequently green light to deactivate the TRPA1 channel. In another aspect, the TRPA1 channel is a Trpa1b channel. In another aspect, the TRPA1 channel is a vertebrate Trpa1b channel. In another aspect, the TRPA1 channel is a zebrafish (Danio rerio) Trpa1b channel. In another aspect, activation of the TRPA1 channel leads to an increase in current and deactivation leads to a decrease in current.

Another embodiment described herein is a means for the activation or deactivation of a TRPA1 channel comprising contacting a TRPA1 channel with of any of the compounds described herein and pulse illuminating the compound with violet light to activate the TRPA1 channel or subsequently green light to deactivate the Trap1b channel.

Another embodiment described herein is the use of any of the compounds described herein for the reversible activation or deactivation of a TRPA1 channel.

Another embodiment described herein is a method for synthesizing of any of the compounds described herein, the method comprising: (a) reacting a pyrazole amine or a phenyl amine with a diazotizing mixture comprising sodium nitrite and one or more of HCl, H₂SO₄, HBF₄, AcOH, or tosic acid and incubating for a period of time to produce a product; (b) adding benzene-1,3-diamine and sodium acetate in a methanol/water mixture to the product of (a); (c) performing an organic extraction and purifying the product of (b); (d) combining the purified product of (c) in pyridine with propylphosphonic anhydride (T3P) in ethyl acetate and heating for a period of time to produce a product; and (e) performing an organic extraction and purifying the product of (d); or (a1) reacting aniline with potassium peroxymonosulfate in a biphasic mixture of organic solvent and water under an oxygen free atmosphere and incubating for a period of time at room temperature to produce at product; (b1) performing an organic extraction of the product of (a1) to form an extracted product; (c1) reacting a nitrobenzene amine with either 2-furoyl chloride or 2-thiophenecarbonyl chloride and heating for a period of time to produce a product; (d1) purifying the product of (c1); (e1) combining the purified product of (d1) with a mixture of organic solvent, iron and an aqueous solution of ammonium chloride, and heating for a period of time to produce a product; (f1) purifying the product of (e1); (g1) reacting the purified product of (f1) with the extracted product of (b1) in an acid and an organic solvent and heating for a period of time to produce a product; and (h1) performing an organic extraction and purifying the product of (g1); or (a2) reacting an azobenzene amine with either 2-furoic acid or 2-thiophenecarboxylic acid in pyridine and propylphosphonic anhydride (T3P) in ethyl acetate and heating for a period of time to produce a product; and (b2) performing an organic extraction and purifying the product of (a2).

Another embodiment described herein is a reversible photoswitch compound synthesized by any of the methods described herein.

Another embodiment described herein is a kit comprising two or more of: Compound 9, a Trpa1b plasmid (pCMV-zTrpa1b-FLAG; SEQ ID NO:3); Tol2-ngn1-Trpa1b-2A-mCherry (partial vector sequence in SEQ ID NO:5); a zebrafish Trpa1b^{-/-} embryo; a HEK293T cell expressing zebrafish Trpa1b; transfection reagents; buffers and reagents; a light source capable of illuminating in the violet and green wavelengths; packaging, containers, and instructions for use.

### DESCRIPTION OF THE DRAWINGS

FIG. 1 shows the crystal structure of TRPswitch-B (50% probability ellipsoids).

FIG. 2 shows that photoswitching of TRPswitch-A activates the Trpa1b channel. FIG. 2A shows a schematic diagram of the behavioral screening assay setup. Screening was performed using a 96-well plate format. Three wild-type zebrafish larvae at 3 days post fertilization (dpf) were placed in each well. Individual small molecules were added to each well. A series of different wavelengths of light from WL1-WL4 were applied to each well and the motion activity from the well was recorded. WL1, 450-500 nm; WL2, 415-455 nm; WL3, 352-402 nm; WL4, white light. The screening assay was performed on a motorized inverted compound microscope. A hit is defined as having motion in the well after photoactivation. It also shows an image of one well on the 96-well plate with 3 zebrafish larvae. FIG. 2B is a line graph showing the zebrafish behavioral response of TRPswitch-A treated animals. The vertical bars indicate the timing of a 1 s light stimulus at the indicated wavelength. FIG. 2C shows the electrophysiology analysis of TRPswitch-A in HEK293T cells expressing zebrafish Trpa1b. Current density-Voltage relationships of Trpa1b current without treatment (black trace), 10 μM TRPswitch-A with violet light stimulus (grey dotted trace), and then followed by green light stimulus (grey trace). FIG. 2D shows isomerization of TRPswitch-A as indicated by the absorbance profile of TRPswitch before irradiation (black dotted line), after violet light (352-402 nm) irradiation (grey dotted line), and subsequently after green light (500-600 nm) irradiation (grey line). FIG. 2E shows basal zTrpa1b currents (black trace) are activated (grey dotted trace) and subsequently desensitize (grey trace) with AITC.

FIG. 3 shows the TRPswitch structure-activity relationship analysis. FIG. 3A shows the DMSO control and Compounds 1-4. FIG. 3B shows Compounds 5-9. The structure of individual compounds and their in vivo activity are shown in the bar graph side by side for comparison. The assay was performed as described in FIG. 2. Each bar in the graph represents the motion response after photoactivation using individual wavelength sets as indicated in the lower x-axis. WL1, 450-500 nm; WL2, 415-455 nm; WL3, 352-402 nm; WL4, white light. 1% DMSO was used as a control. Values are means±SEM from at least 3 experimental setups. Compounds with robust biological activity are labeled.

FIG. 4 shows the Properties of TRPswitch. FIG. 4A shows the thermal kinetics of TRPswitch-A (Compound 1) and TRPswitch-B (Compound 9) at room temperature. The natural log of the Z isomer percentage is plotted against time. FIG. 4B shows that the activity of TRPswitch requires Trpa1b. Bar graph showing the average light-induced motion response of the different treatment groups. WT, Wild-type; Trpa1b^{-/-}, Trpa1b mutant. Values are means±SEM from 3 experiments. FIG. 4C is a line graph indicating the positive correlation between the percentage of wild-type larvae that were pretreated with TRPswitch and showed light-induced motion response with increasing duration of light stimulus. Values are means±SEM from 3 experiments. FIG. 4D is a bar graph showing the response latency (time from the beginning of light pulse to the first motion response) with various intensities of light stimulation as indicated. Values are means±SEM 3 experiments. FIG. 4E is a dose response curve showing TRPswitch's effects on animal behavior (n=3). FIG. 4F shows the isomerization of TRPswitch-B as indicated by the absorbance profile of TRPswitch before irradiation (black dotted line), after violet light (352-402 nm) irradiation (grey dotted line), and subsequently after green light (500-600 nm) irradiation (grey line). FIG. 4G and FIG. 4H show electrophysiology analysis of TRPswitch. Representative peak whole cell Trpa1b current densities measured at −100 mV (grey trace) and +100 mV (black trace) with TRPswitch-A (Compound 1) (FIG. 4G) and TRPswitch-B (Compound 9) (FIG. 4H) treatment. Violet light and green light illumination were applied at the time indicated by the light grey and dark grey vertical boxes, respectively. FIG. 4I shows that the basal zTrpa1b current density (dark grey) increases with light activated TRPswitches or the positive control, AITC (light grey). Values are means±SEM taken at +100 or −100 mV. *<0.05. FIG. 4J shows the mechanism by which TRPswitch activates Trpa1b is distinct from optovin. DABCO abolished optovin/light-induced motion response but had no effect on TRPswitch-B. Values are means±SEM from at least 3 experiments. ****<0.005.

FIG. 5A shows the thermal isomerisation kinetics for TRPswitch-A (Compound 1) at 25° C. in 30% water:DMSO.

FIG. 5B shows the thermal isomerisation kinetics for TRPswitch-B (Compound 9) at 25° C. in 30% water:DMSO.

FIG. 6A shows the UV-Vis spectra of TRPswitch-A (Compound 1) in 30% water:DMSO at various PSS, including estimated pure Z-spectrum.

FIG. 6B shows the UV-Vis spectra of TRPswitch-B (Compound 9) in 30% water:DMSO at various PSS, including estimated pure Z-spectrum.

FIG. 7 shows TRPswitch-A (Compound 1) 420 nm PSS with relevant peaks for E and Z isomers labelled.

FIG. 8 shows TRPswitch-A (Compound 1) 365 nm PSS with relevant peaks for E and Z isomers labelled.

FIG. 9 shows TRPswitch-A (Compound 1) 495 nm PSS with relevant peaks for E and Z isomers labelled.

FIG. 10 shows TRPswitch-B (Compound 9) 420 nm PSS with relevant peaks for E and Z isomers labelled.

FIG. 11 shows TRPswitch-B (Compound 9) 365 nm PSS with relevant peaks for E and Z isomers labelled.

FIG. 12 shows TRPswitch-B (Compound 9) 495 nm PSS with relevant peaks for E and Z isomers labelled.

FIG. 13 shows the reversible and repeatable activity of TRPswitch. FIG. 13A-FIG. 13D show light-controlled heartbeat interruption in zebrafish larvae in vivo. Experiments were performed on Trpa1b^{-/-} larvae expressing Trpa1b in cardiomyocytes. Larvae were pre-incubated with TRPswitch for 1 h in the dark before experimental manipulation. FIG. 13A shows an image of zebrafish heart showing the ventricle chamber and the position where ventricle width measurements were made and displayed in FIG. 13B. FIG. 13B shows a representative line graph showing the ventricle width over time in larvae treated with TRPswitch-B. The dark grey and light grey vertical rectangles indicate photoactivation with a 1 s pulse of violet light and green light, respectively. FIG. 13C and FIG. 13D show repetitive stopping and re-starting of ventricle heart contractions by violet and green light, respectively. Dotted plot showing the ventricle contraction frequency in individual larvae illuminated with a series of light flashes as indicated in the x-axis. Each dot represents measurement from one larva. Larvae were treated with 20 μM TRPswitch-A (FIG. 13C) or TRPswitch-B (FIG. 13D). FIG. 13E shows that TRPswitch induced a sustained heart contraction after a brief pulse of violet light. Transgenic zebrafish expressing Trpa1b in cardiomyocytes were pre-treated with 20 μM TRPswitch. A 1 s pulse of violet light was applied to the heart and the ventricle heart diastolic width ratio (comparing the diastolic width before and after photoactivation) was quantified. Values are means±SEM from 3 experiments.

FIG. 14 shows heterologous utility of zebrafish Trpa1b/TRPswitch. FIG. 14A shows whole cell current measurement in untransfected CCD-18Co cells with endogenous human TRPA1 expression. Current density-Voltage relationship of human TRPA1 current with AITC (light grey).

FIG. 14B shows whole cell current measurement in untransfected CCD-18Co cells in the presence of TRPswitch-B and after violet and green light illumination. FIG. 14C shows whole cell current measurement in CCD-18Co cells transfected with zebrafish Trpa1b. Current density-Voltage relationship of zebrafish Trpa1b current in the presence of TRPswitch-B and after violet or green light illumination.

### DETAILED DESCRIPTION

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are those that are well known and commonly used in the art. In case of conflict, the present document, including definitions, will control. Preferred methods and materials are described below, although methods and materials similar or equivalent to those described herein can be used in practice or testing of the present invention.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more than 3 standard deviations, per the practice in the art. Alternatively, such as with respect to biological systems or processes, the term “about” can mean within an order of magnitude, in some embodiments within 5-fold, and in some embodiments within 2-fold, of a value.

All ranges disclosed herein include both end points as discrete values as well as all integers and fractions specified within the ranges. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points. As used herein, the symbol “˜” means “about.” As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” “reference level,” and “reference” are used herein interchangeably. The reference level may be a predetermined value or range, which is employed as a benchmark against which to assess the measured result. “Control group” as used herein refers to a group of control subjects.

As used herein, the term “dose” denotes any form of the active ingredient formulation or composition that contains an amount sufficient to produce a therapeutic effect with at least a single administration. “Formulation” and “composition” are used interchangeably herein.

As used herein, the term “prophylaxis” refers to preventing or reducing the progression of a disorder, either to a statistically significant degree or to a degree detectable to one skilled in the art.

As used herein, the terms “effective amount” or “therapeutically effective amount,” refers to a substantially non-toxic, but sufficient amount of an agent or a composition being administered that will prevent, treat, or ameliorate to some extent one or more of the symptoms of the disease or condition being treated. The result can be reduction or alleviation of the signs, symptoms, or causes of a disease, or any other desired alteration of a biological system. An effective amount may be based on factors individual to each patient, including, but not limited to, the patient's age, size, type or extent of disease, stage of the disease, route of administration, the type or extent of supplemental therapy used, ongoing disease process and type of treatment desired.

As used herein, “sample” or “test sample” as used herein can mean any sample in which the presence and/or level of a target is to be detected or determined or any sample comprising a compound or ion channel, or component thereof as described herein. Samples may include liquids, solutions, emulsions, or suspensions. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. The sample can be used directly as obtained from a subject or can be pre-treated, such as by filtration, distillation, extraction, concentration, centrifugation, inactivation of interfering components, addition of reagents, and the like, to modify the character of the sample in some manner as discussed herein or otherwise as is known in the art.

As used herein, the term “subject” refers to an animal. Typically, the animal is a mammal. A subject also refers to, for example, primates (e.g., humans, male or female; infant, adolescent, or adult), pigs, cows, sheep, goats, horses, dogs, cats, rabbits, rats, mice, fish, birds, and the like. In one embodiment, the subject is a human. In another embodiment, the subject is a fish.

As used herein, a subject is “in need of” a treatment if such subject would benefit biologically, medically, or in quality of life from such treatment.

As used herein, the terms “inhibit,” “inhibition,” or “inhibiting” refer to the reduction or suppression of a given condition, symptom, or disorder, or disease, or a significant decrease in the baseline activity of a biological activity or process.

As used herein, “treatment” or “treating” refers to means suppressing, repressing, reversing, alleviating, ameliorating, or inhibiting the progress of disease, or completely eliminating a disease. A treatment may be either performed in an acute or chronic way. The term also refers to reducing the severity of a disease or symptoms associated with such disease prior to affliction with the disease. Preventing the disease involves administering a composition or compound of the present invention to a subject prior to onset of the disease. Suppressing the disease involves administering a composition or compound of the present invention to a subject after induction of the disease but before its clinical appearance. Repressing or ameliorating the disease involves administering a composition or compound of the present invention to a subject after clinical appearance of the disease.

Definitions of specific functional groups and chemical terms are described in more detail herein. The chemical elements are identified in accordance with the Periodic Table of the Elements, CAS version, Handbook of Chemistry and Physics, 75^{th} ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} ed, John Wiley & Sons, Inc., New York, 2001; Larock, Comprehensive Organic Transformations, VCH Publishers, Inc., New York, 1989; and Carruthers, Some Modern Methods of Organic Synthesis, 3rd ed, Cambridge University Press, Cambridge, 1987.

As used herein, the term “alkyl” refers to a radical of a straight chain or branched saturated hydrocarbon group having from 1 to 6 carbon atoms (“C₁₋₆ alkyl”). In some embodiments, an alkyl group has 1 to 5 carbon atoms (“C₁₋₆ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C_{2-g} alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tertiary amyl), and hexyl (C₆) (e.g., n-hexyl).

As used herein, the term “alkylene” refers to a divalent radical of an alkyl group, e.g., —CH₂—, —CH₂CH₂—, and —CH₂CH₂CH₂—.

As used herein, the term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 10 carbon atoms and 1 or more heteroatoms within the parent chain (“heteroC₁₋₁₀ alkyl”). In certain embodiments, the heteroalkyl group is an unsubstituted heteroC₁₋₁₀ alkyl. In certain embodiments, the heteroalkyl group is a substituted heteroC₁₋₁₀ alkyl.

As used herein, the term “heteroalkylene” refers to a divalent radical of a heteroalkyl group.

As used herein, the terms “alkoxy” or “alkoxyl” refers to an —O-alkyl radical. In some embodiments, the alkoxy groups are methoxy, ethoxy, n-propoxy, isopropoxy, n-butoxy, tert-butoxy, sec-butoxy, n-pentoxy, n-hexoxy, and 1,2-dimethylbutoxy. In some embodiments, alkoxy groups are lower alkoxy, i.e., with between 1 and 6 carbon atoms. In some embodiments, alkoxy groups have between 1 and 4 carbon atoms.

As used herein, the term “aryl” refers to a stable, aromatic, mono- or bicyclic ring radical having the specified number of ring carbon atoms. Examples of aryl groups include, but are not limited to, phenyl, 1-naphthyl, 2-naphthyl, and the like. The related term “aryl ring” likewise refers to a stable, aromatic, mono- or bicyclic ring having the specified number of ring carbon atoms.

As used herein, the term “heteroaryl” refers to a stable, aromatic, mono- or bicyclic ring radical having the specified number of ring atoms and comprising one or more heteroatoms individually selected from nitrogen, oxygen, or sulfur. The heteroaryl radical may be bonded via a carbon atom or heteroatom. Examples of heteroaryl groups include, but are not limited to, furyl, pyrrolyl, thienyl, pyrazolyl, imidazolyl, thiazolyl, isothiazolyl, oxazolyl, isoxazolyl, triazolyl, tetrazolyl, pyrazinyl, pyridazinyl, pyrimidyl, pyridyl, quinolinyl, isoquinolinyl, indolyl, indazolyl, oxadiazolyl, benzothiazolyl, quinoxalinyl, and the like. The related term “heteroaryl ring” likewise refers to a stable, aromatic, mono- or bicyclic ring having the specified number of ring atoms and comprising one or more heteroatoms individually selected from nitrogen, oxygen, or sulfur. In certain embodiments, the heteroaryl group is an unsubstituted heteroaryl. In some embodiments, the heteroaryl group is a substituted heteroaryl. The substitution can be on any atom with the ability to accept a substitution.

As used herein, the term “carbocyclyl” refers to a stable, saturated, or unsaturated, non-aromatic, mono- or bicyclic (fused, bridged, or spiro) ring radical having the specified number of ring carbon atoms. Examples of carbocyclyl groups include, but are not limited to, the cycloalkyl groups identified above, cyclobutenyl, cyclopentenyl, cyclohexenyl, and the like. In an embodiment, the specified number is C₃-C₁₂ carbons. The related term “carbocyclic ring” likewise refers to a stable, saturated, or unsaturated, non-aromatic, mono- or bicyclic (fused, bridged, or spiro) ring having the specified number of ring carbon atoms.

As used herein, the term “heterocyclyl” refers to a stable, saturated or unsaturated, non-aromatic, mono- or bicyclic (fused, bridged, or spiro) ring radical having the specified number of ring atoms and comprising one or more heteroatoms individually selected from nitrogen, oxygen and sulfur. The heterocyclyl radical may be bonded via a carbon atom or heteroatom. In an embodiment, the specified number is C₃-C₁₂ carbons. Examples of heterocyclyl groups include, but are not limited to, azetidinyl, oxetanyl, pyrrolinyl, pyrrolidinyl, tetrahydrofuryl, tetrahydrothienyl, piperidyl, piperazinyl, tetrahydropyranyl, morpholinyl, perhydroazepinyl, tetrahydropyridinyl, tetrahydroazepinyl, octahydropyrrolopyrrolyl, and the like. The related term “heterocyclic ring” likewise refers to a stable, saturated or unsaturated, non-aromatic, mono- or bicyclic (fused, bridged, or spiro) ring having the specified number of ring atoms and comprising one or more heteroatoms individually selected from nitrogen, oxygen and sulfur.

As used herein the terms “halogen” or “halo” refers to fluorine (fluoro, —F), chlorine (chloro, —Cl), bromine (bromo, —Br), or iodine (iodo, —I).

As used herein the term “substituted,” whether preceded by the term “optionally” or not, means that one or more hydrogens of the designated moiety are replaced with a suitable substituent. Exemplary substitutents include H, halogen, C₁₋₆ alkyl, C₁₋₆ alkenyl, C₁₋₆ alkynyl, C₁₋₆ alkoxy, C₀₋₆ amine, C₀₋₆ amide, C₀₋₆—OH, C₀₋₆—COOH, or C₀₋₆ CN.

As used herein, the definition of each expression, e.g., alkyl, m, n, etc., when it occurs more than once in any structure, is intended to be independent of its definition elsewhere in the same structure.

Described herein are TRPswitch compounds and methods for activating and deactivating ion channels with such compounds.

A customized, light-responsive chemical library coupled with a behavior-based screening assay with zebrafish larvae was used to discover “TRPswitch” azoarene photoswitchable ligands for the TRPA1 channel. TRPswitch molecules allow for optical control of both the activation and deactivation of the TRPA1 channel. This analysis suggests that the zebrafish Trpa1b channel is necessary and sufficient for TRPswitch light-induced activity. Channel activation and deactivation can be controlled by violet light and green light illumination, respectively. The TRPswitch/light induced TRPA1 channel activity is reversible and repeatable in vivo, and sustained channel activation is achieved after only a short pulse of light illumination. This appears to be the first described photoswitchable TRPA1 system. These data show that this TRPA1/TRPswitch system is a robust chemo-optogenetic tool that can be applied to both neuronal and non-neuronal cells. The TRPA1/TRPswitch system's step function properties, along with its high unitary conductance, make it a complementary alternative to existing chemo-optogenetic tools.

Using a zebrafish behavior-based screening strategy, “TRPswitch,” a photoswitchable non-electrophilic ligand scaffold for the TRPA1 channel was discovered. TRPA1 exhibits high unitary channel conductance, making it an ideal target for chemo-optogenetic tool development. Key molecular determinants for the activity of TRPswitch were elucidated and allowed for replacement of the TRPswitch azobenzene with a next generation azoheteroarene. The TRPswitch compounds enable reversible, repeatable, and nearly quantitative light-induced activation and deactivation of the vertebrate TRPA1 channel with violet and green light, respectively. The utility of TRPswitch compounds was demonstrated in larval zebrafish hearts exogenously expressing zebrafish Trpa1b, where heartbeat could be controlled using TRPswitch and light. Therefore, TRPA1/TRPswitch represents a novel step-function chemo-optogenetic system with a unique combination of high conductance, high efficiency, activity against an unmodified vertebrate channel, and capacity for bidirectional optical switching. This chemo-optogenetic system is particularly applicable in systems where a large depolarization current is needed, or sustained channel activation is desirable.

One embodiment described herein is a photoactive compound of Formula (I):

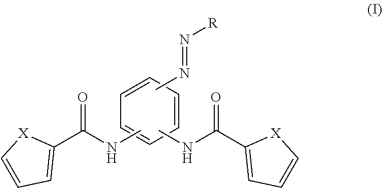

wherein each X is independently O or S and R is a substituted or unsubstituted heteroaryl moiety or a substituted phenyl moiety.

In another embodiment, the compound is one of Formulae (II), (Ill), or (IV):

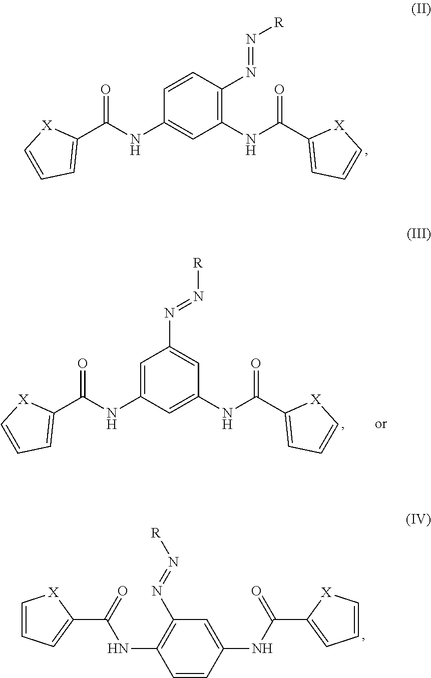

wherein each X is independently O or S and R is a substituted or unsubstituted heteroaryl moiety or a substituted phenyl moiety. In one aspect, each X is independently O or S and R is a mono- or bi-cyclic aryl ring or a 5-10 membered mono or bi-cyclic heteroaryl ring optionally substituted with one or more of Q₁-(R₁)ₙ; Q₁ is a covalent bond, H, O, halogen, cyano, —NR₃—, —CONR₂—, —NR₂CO—, oxo, nitro, —S(O)ₘ—, —C₁₋₆ haloalkyl, —C₁₋₆ alkoxy, —C₁₋₆ haloalkoxy, —C₁₋₆ hydroxyalkyl, —C₁₋₆ cyanoalkyl, —CO—, —SO₂R₃, —NR₃R₄, —NR₃COR₄, —NR₂CONR₃R₄, —CONR₃R₄, —CO₂R₃, —NR₃CO₂R₄, —SO₂NR₃R₄, —CONR₃, —C(O)R₃, —NR₃SO₂R₄, —NR₂SO₂NR₃R₄, —SO₂NR₃, optionally substituted —C₁₋₆ alkylene, optionally substituted —C₂₋₆ alkenylene, or optionally substituted —C₁₋₆ alkyl; R₁ is halogen, oxo, cyano, nitro, optionally substituted —C₁₋₆ haloalkyl, optionally substituted —C₁₋₆ alkoxy, optionally substituted —C₁₋₆ haloalkoxy, optionally substituted —C₁₋₆ alkyl, optionally substituted —C₂₋₆ alkenyl, optionally substituted —C₂₋₆ alkynyl, —C₁-C₆ hydroxyalkyl, optionally substituted heterocyclyl, optionally substituted cycloalkyl, optionally substituted heteroaryl, optionally substituted aryl, -Q₂-NR₅CONR₆R₇, -Q₂-NR₅R₆, -Q₂-NR₅COR₆, -Q₂-COR₅, -Q₂-SO₂R₅, -Q₂—CONR₅, -Q₂-CONR₅R₆, -Q₂-CO₂R₅, -Q₂-SO₂NR₅R₆, -Q₂-NR₅SO₂R₆, or -Q₂-NR₅SO₂NR₆R₇; Q₂ is a covalent bond, —C₁₋₆ alkyl, —C₁₋₆ alkylene, or —C₂₋₆ alkenylene; R₂, R₃, and R₄ are each independently hydrogen, optionally substituted —C₁₋₆ alkyl, or optionally substituted —C₁₋₆ alkylene; R₅, R₆, and R₇ are each independently H, optionally substituted —C₁₋₆ alkyl, optionally substituted heterocyclyl, optionally substituted heteroaryl, optionally substituted aryl, or optionally substituted cycloalkyl; n is 0, 1, 2, 3, or 4; when n is 1, 2, 3, or 4, R₁ is an optionally substituted 3-10 membered heterocyclyl, heteroaryl, aryl, or a mono- or bi-cycloalkyl ring; and wherein n is 0, Q is present and R₁ is absent; m is 0, 1, or 2; and any of the compounds designated as “optionally substituted” may be substituted with halogen, —C₁₋₆ alkyl, —C₁₋₆ alkenyl, —C₁₋₆ alkynyl, —C₁₋₆ alkoxy, —C₀₋₆ amine, —C₀₋₆ amide, —C₀₋₆—OH, —C₀₋₆—COOH, —C₀₋₆ CN, or C₁₋₆ halogen. In one aspect, each X is S. In another aspect, each X is O.

In another embodiment, the compound is Formula (V):

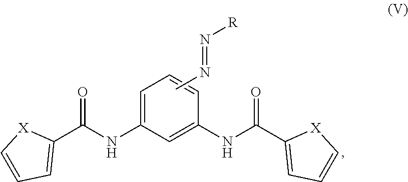

wherein each X is S or O; R is

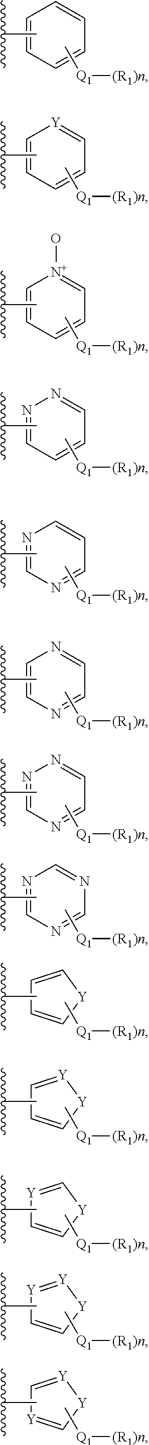

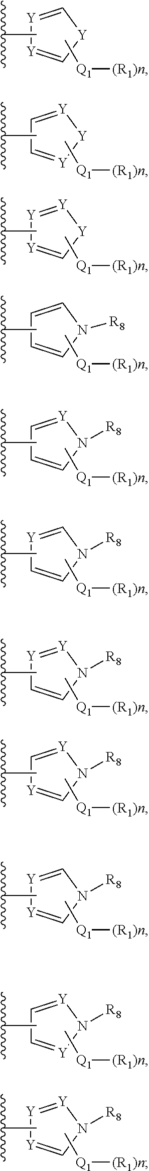

Y is independently O, S, or N; Q₁ is a covalent bond, H, O, halogen, cyano, —NR₃—, —CONR₂—, —NR₂CO—, oxo, nitro, —S(O)ₘ—, C₁₋₆ haloalkyl, C₁₋₆ alkoxy, C₁₋₆ haloalkoxy, C₁₋₆ hydroxyalkyl, C₁₋₆ cyanoalkyl, —CO—, —SO₂R₃, —NR₃R₄, —NR₃COR₄, —NR₂CONR₃R₄, —CONR₃R₄, —CO₂R₃, —NR₃CO₂R₄, —SO₂NR₃R₄, —CONR₃, —C(O)R₃, —NR₃SO₂R₄, —NR₂SO₂NR₃R₄, —SO₂NR₃, optionally substituted —C₁₋₆ alkylene, optionally substituted —C₂₋₆ alkenylene, or optionally substituted —C₁₋₆ alkyl; R₁ is halogen, oxo, cyano, nitro, optionally substituted —C₁₋₆ haloalkyl, optionally substituted —C₁₋₆ alkoxy, optionally substituted —C₁₋₆ haloalkoxy, optionally substituted —C₁₋₆ alkyl, optionally substituted —C₂₋₆ alkenyl, optionally substituted —C₂₋₆ alkynyl, —C₁-C₆ hydroxyalkyl, optionally substituted heterocyclyl, optionally substituted cycloalkyl, optionally substituted heteroaryl, optionally substituted aryl, -Q₂-NR₅CONR₆R₇, -Q₂-NR₅R₆, -Q₂-NR₅COR₆, -Q₂-COR₅, -Q₂-SO₂R₅, -Q₂-CONR₅, -Q₂-CONR₅R₆, -Q₂-CO₂R₅, -Q₂-SO₂NR₅R₆, -Q₂-NR₅SO₂R₆, or -Q₂-NR₅SO₂NR₆R₇; Q₂ is a covalent bond, —C₁₋₆ alkyl, —C₁₋₆ alkylene, or —C₂₋₆ alkenylene; R₂, R₃, and R₄ are each independently hydrogen, optionally substituted —C₁₋₆ alkyl, or optionally substituted —C₁₋₆ alkylene; R₅, R₆, and R₇ are each independently H, optionally substituted —C₁₋₆ alkyl, optionally substituted heterocyclyl, optionally substituted heteroaryl, optionally substituted aryl, or optionally substituted cycloalkyl; R₃ is a covalent bond, hydrogen, halogen, oxygen, oxo, nitro, cyano, —NR₃—, —CONR₃—, —NR₃CO—, —S(O)ₘ—, C₁-C₆ haloalkyl, —C₁-C₆ alkoxy, —C₁-C₆ haloalkoxy, —C₁-C₆ hydroxyalkyl, —C₁-C₆ cyanoalkyl, —CO—, —SO₂R₄, —NR₃R₄, —NR₃COR₄, —NR₂CONR₃R₄, —CONR₃R₄, —CO₂R₃, —NR₃CO₂R₄, —SO₂NR₃R₄, —CONR₃, —C(O)R₃, —NR₃SO₂R₄, —NR₂SO₂NR₃R₄, —SO₂NR₃, optionally substituted C₁-C₆ alkylene, optionally substituted —C₂-C₆ alkenylene, or optionally substituted —C₁-C₆ alkyl; n is 0, 1, 2, 3, or 4; when n is 1, 2, 3, or 4, R₁ is an optionally substituted 3-10 membered heterocyclyl, heteroaryl, aryl, or a mono- or bi-cycloalkyl ring; and wherein n is 0, Q is present and R₁ is absent; m is 0, 1, or 2; and any of the compounds designated as “optionally substituted” may be substituted with halogen, —C₀₋₆ alkyl, —C₁₋₆ alkenyl, —C₁₋₆ alkynyl, —C₁₋₆ alkoxy, —C₀₋₆ amine, —C₀₋₆ amide, —C₀₋₆—OH, —C₀₋₆—COOH, —C₀₋₆ CN, or C₁₋₆ halogen.
In one aspect, each X is S or O and R is:

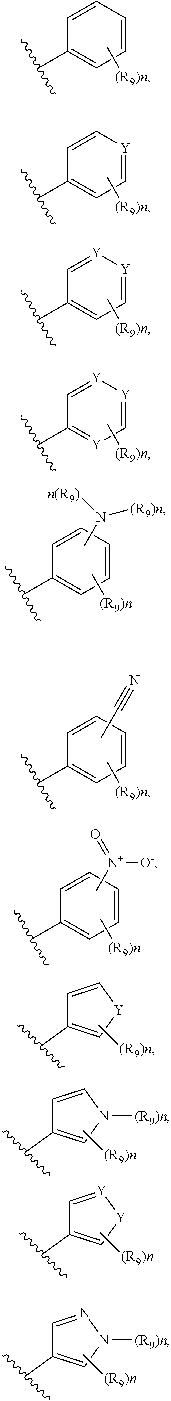

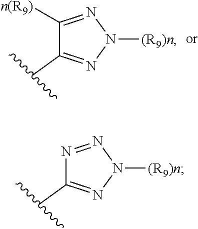

Y is independently O, S, or N; R₉ is independently H, halogen, C₁₋₆ alkyl, C₁₋₆ alkenyl, C₁₋₆ alkynyl, C₁₋₆ alkoxy, C₀₋₆ amine, C₀₋₆ amide, C₀₋₆—OH, C₀₋₆—COOH, C₀₋₆ CN, C₁₋₆ halogen, or —CF₃; and n is 0, 1, 2, 3, or 4. In another aspect, each X is S or O and R is:

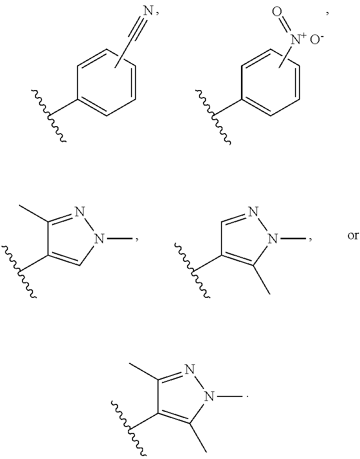

In another aspect, each X is S or O and R is:

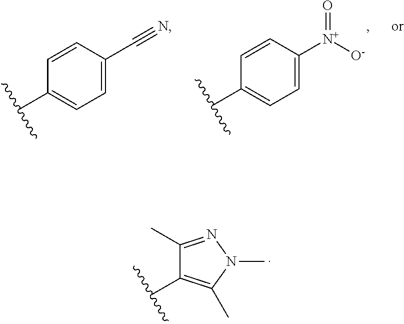

In another embodiment, the compound is selected from:

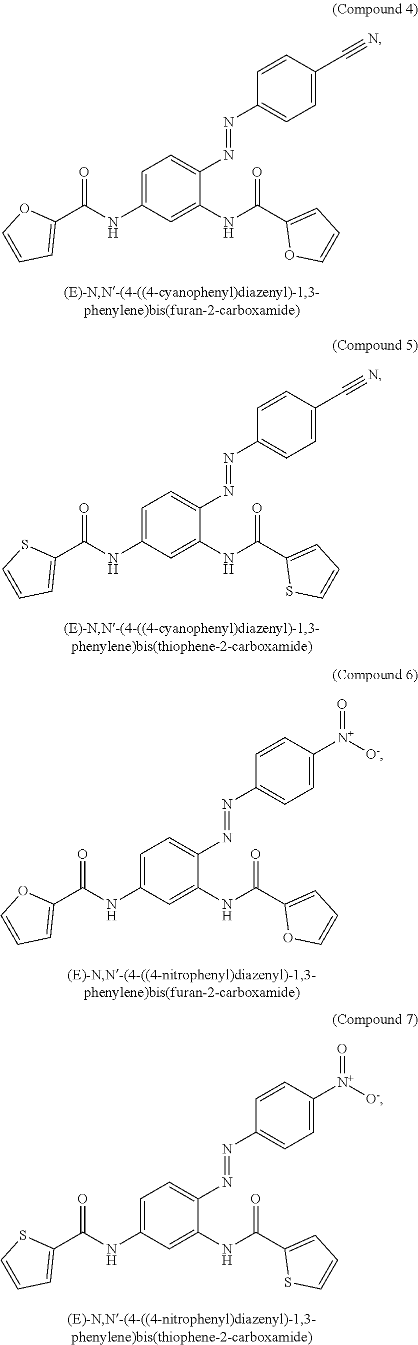

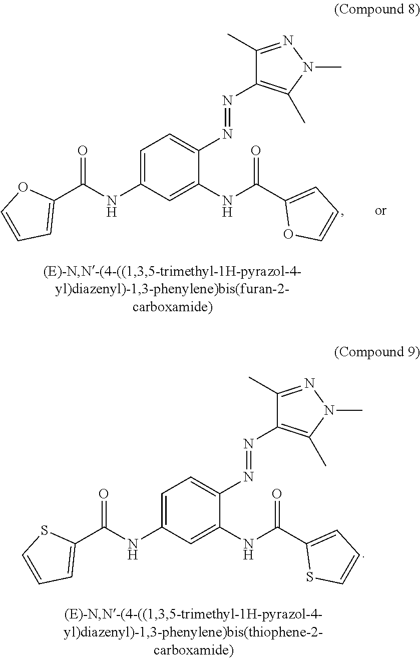 [SMILES: Cc1nn(C)c(C)c1/N=N/c1ccc(NC(=O)c2ccco2)cc1NC(=O)c1ccco1 ; Cc1nn(C)c(C)c1/N=N/c1ccc(NC(=O)c2cccs2)cc1NC(=O)c1cccs1]

In another aspect, each X is S or O, and R is a substituted or unsubstituted arylazopyrazole.
In another aspect, each X is S or O and R is

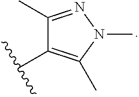

In another embodiment, the compound is:

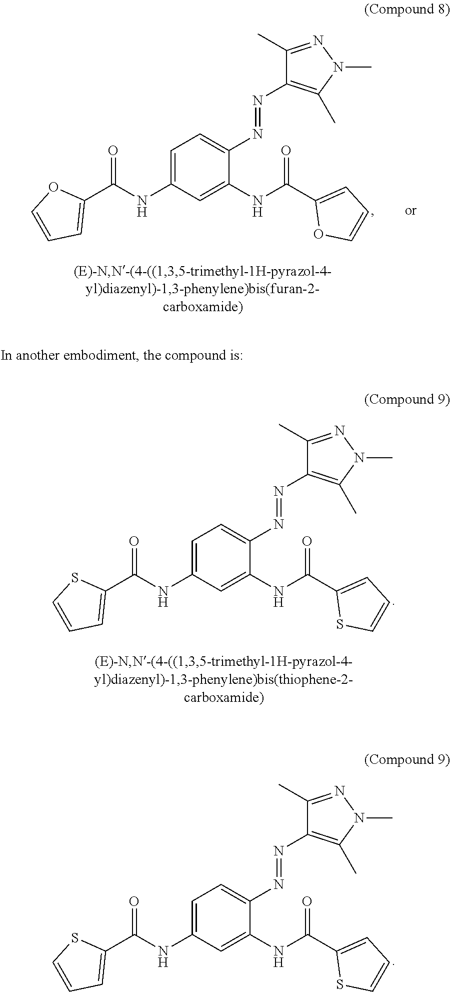 [SMILES: Cc1nn(C)c(C)c1/N=N/c1ccc(NC(=O)c2ccco2)cc1NC(=O)c1ccco1 ; Cc1nn(C)c(C)c1/N=N/c1ccc(NC(=O)c2cccs2)cc1NC(=O)c1cccs1 ; Cc1nn(C)c(C)c1/N=N/c1ccc(NC(=O)c2cccs2)cc1NC(=O)c1cccs1]

The disclosed compounds may exist as a pharmaceutically acceptable salt. The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compound or separately by reacting an amino group of the compound with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthalenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric and the like. The amino groups of the compound may also be quaternized with alkyl chlorides, bromides, and iodides such as methyl, ethyl, propyl, isopropyl, butyl, lauryl, myristyl, stearyl and the like.

Basic addition salts may be prepared during the final isolation and purification of the disclosed compound by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, N,N′-dibenzylethylenediamine, ethylenediamine, ethanolamine, diethanolamine, piperidine, piperazine, and the like.

Another embodiment described herein is a compound described herein that is a reversible photoswitch that acts on a TRPA1 channel.

Another embodiment described herein is a research tool comprising any of the compounds described herein.

Another embodiment described herein is a method for reversibly activating or deactivating a TRPA1 channel, the method comprising: contacting a TRPA1 channel with an E isomer of any of the compounds described herein; pulse illuminating the compound with violet light (˜350-405 nm) to induce an E→Z isomerization and activate the TRPA1 channel; and subsequently, pulse illuminating the compound with green light (˜500-600 nm) to induce a Z→E isomerization and deactivate the TRPA1 channel. In one aspect, the compound is:

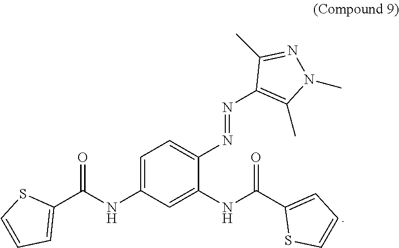 [SMILES: Cc1nn(C)c(C)c1/N=N/c1ccc(NC(=O)c2cccs2)cc1NC(=O)c1cccs1]

In another aspect, the compound has a concentration of about 10-20 μM. In another aspect, the compound is administered to an organism, part thereof, or cell culture, and the organism, part thereof, or cell culture is pulse illuminated with violet light to activate and subsequently green light to deactivate the TRPA1 channel. In another aspect, the TRPA1 channel is a Trpa1b channel. In another aspect, the TRPA1 channel is a vertebrate Trpa1b channel. In another aspect, the TRPA1 channel is a zebrafish (Danio rerio) Trpa1b channel. In another aspect, activation of the TRPA1 channel leads to an increase in current and deactivation leads to a decrease in current.

Another embodiment described herein is a means for the activation or deactivation of a TRPA1 channel comprising contacting a TRPA1 channel with of any of the compounds described herein and pulse illuminating the compound with violet light to activate the TRPA1 channel or subsequently green light to deactivate the Trap1b channel.

Another embodiment described herein is the use of any of the compounds described herein for the reversible activation or deactivation of a TRPA1 channel.

Another embodiment described herein is a method for synthesizing of any of the compounds described herein, the method comprising: (a) reacting a pyrazole amine or a phenyl amine with a diazotizing mixture comprising sodium nitrite and one or more of HCl, H₂SO₄, HBF₄, AcOH, or tosic acid and incubating for a period of time to produce a product; (b) adding benzene-1,3-diamine and sodium acetate in a methanol/water mixture to the product of (a); (c) performing an organic extraction and purifying the product of (b); (d) combining the purified product of (c) in pyridine with propylphosphonic anhydride (T3P) in ethyl acetate and heating for a period of time to produce a product; and (e) performing an organic extraction and purifying the product of (d); or (a1) reacting aniline with potassium peroxymonosulfate in a biphasic mixture of organic solvent and water under an oxygen free atmosphere and incubating for a period of time at room temperature to produce at product; (b1) performing an organic extraction of the product of (a1) to form an extracted product; (c1) reacting a nitrobenzene amine with either 2-furoyl chloride or 2-thiophenecarbonyl chloride and heating for a period of time to produce a product; (d1) purifying the product of (c1); (e1) combining the purified product of (d1) with a mixture of organic solvent, iron and an aqueous solution of ammonium chloride, and heating for a period of time to produce a product; (f1) purifying the product of (e1); (g1) reacting the purified product of (f1) with the extracted product of (b1) in an acid and an organic solvent and heating for a period of time to produce a product; and (h1) performing an organic extraction and purifying the product of (g1); or (a2) reacting an azobenzene amine with either 2-furoic acid or 2-thiophenecarboxylic acid in pyridine and propylphosphonic anhydride (T3P) in ethyl acetate and heating for a period of time to produce a product; and (b2) performing an organic extraction and purifying the product of (a2).

Another embodiment described herein is a reversible photoswitch compound synthesized by any of the methods described herein.

Another embodiment described herein is a kit comprising two or more of: Compound 9, a Trpa1b plasmid (pCMV-zTrpa1b-FLAG; SEQ ID NO:3); Tol2-ngn1-Trpa1b-2A-mCherry (partial vector sequence in SEQ ID NO:5); a zebrafish Trpa1b^{-/-} embryo; a HEK293T cell expressing zebrafish Trpa1b; transfection reagents; buffers and reagents; a light source capable of illuminating in the violet and green wavelengths; packaging, containers, and instructions for use.

These studies have identified TRPswitch A and B, two photoswitchable small molecules that enable optical control of currents in the Trpa1b expressing cells in vivo. These data suggest that the TRPswitches specifically target Trpa1b channel and enable repeatable optical control of both neuronal and non-neuronal cells. This is the first example of TRPA1 channel activation by a photoswitchable compound. Importantly, the TRPswitches allow for sustained channel activation after only a brief pulse of violet light illumination, but the channel can also be rapidly deactivated with green light illumination. As only short pulses of light are required to control the activity of the TRPA1 channel, cells subjected to the TRPA1/TRPswitch chemo-optogenetic system are less prone to phototoxicity. This requirement of a short photoactivation period for target activation is a unique and advantageous feature among current PCLs for unmodified ion channels [11-15] and receptors [44].

The TRPA1 channel's high conductance combined with the step function property of the TRPA1/TRPswitch chemo-optogenetic system offers certain new opportunities for basic research. As TRPswitch activity is specific to zebrafish Trpa1b, TRPswitch can be used in heterologous applications by expressing zTrpa1b in animals or cells with endogenous mammalian TRPA1 expression, without interference from the endogenous channel. This new tool will be particularly beneficial in applications where a large depolarization current is needed, such as in large primary motor neurons, or when sustained channel activation is desirable. In addition, tools for manipulating TRPA1 activity are relevant for medical research since TRPA1 is involved in inflammatory and neuropathic pain, itch, and respiratory diseases [20-21, 45]. TRPswitch may prove useful as a research tool to help dissect the mechanism of TRPA1-related disease, as well as to identify disease-modifying agents.

The TRPA1/TRPswitch chemo-optogenetic system offers several advantages over existing tools and was shown here to be robust and easy to use in cultured mammalian cells and in zebrafish. TRPswitch is a freely diffusible small molecule and exposing zebrafish larvae by incubating them in a solution containing TRPswitch is sufficient for robust activity. The uptake and distribution in rodents and larger mammals may be similar to that of zebrafish larvae. When the zebrafish larvae were incubated with TRPswitch for several hours, no obvious adverse physiological phenotypes were observed. The TRPswitches show minimal activity on TRPA1 before photoactivation, highlighting the specificity of the light-induced effect of TRPswitch. On the other hand, as the TRPswitches display a wide range of spectral activity, it may be difficult to combine a TRPswitch with other optogenetic tools or fluorescence-based biosensors due to potential spectral overlap. The use of violet light for TRPswitch activation may also limit the tissue depth of its activation; however, multi-photon [46] or longer near infrared (NIR) light excitation [47] may be used.

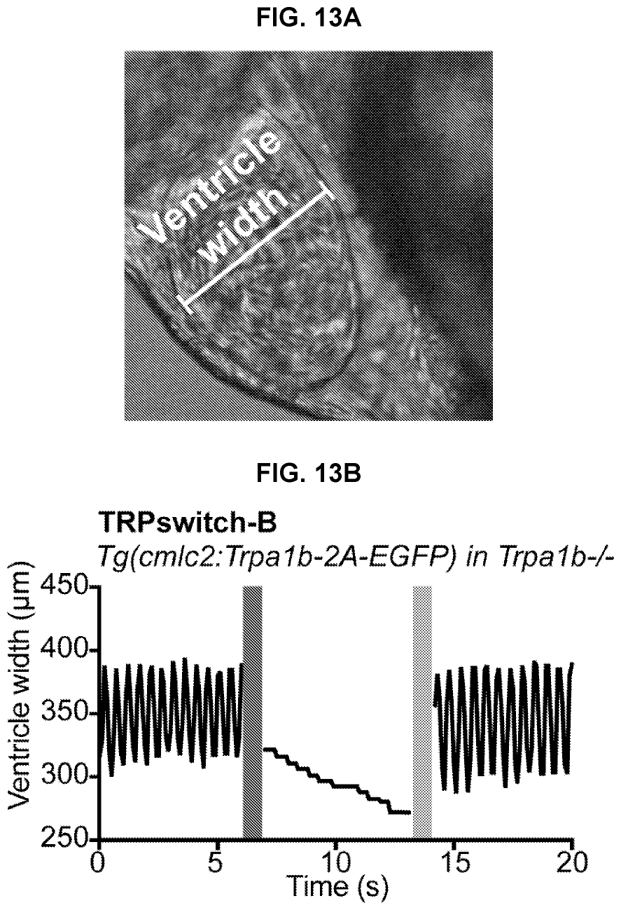

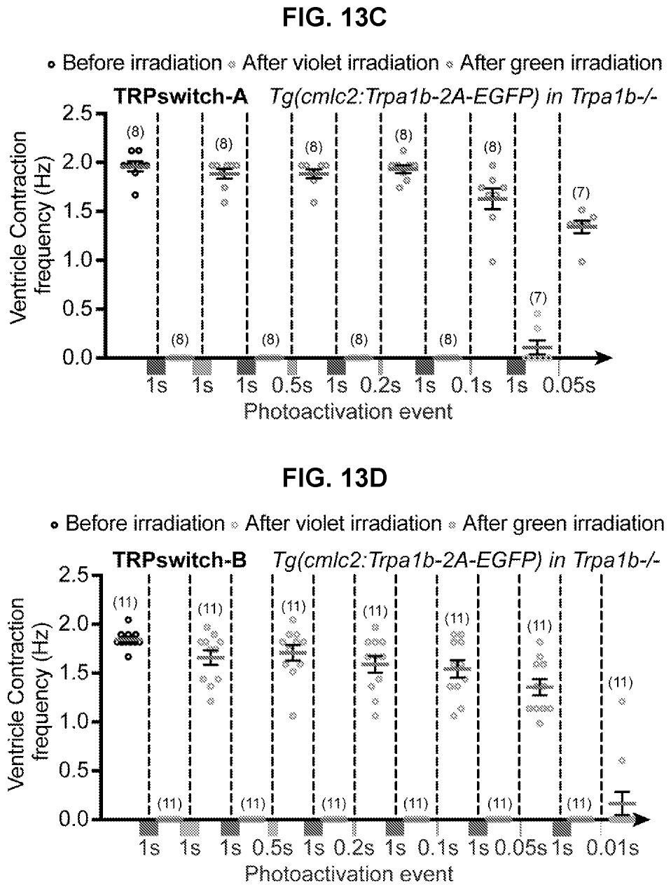

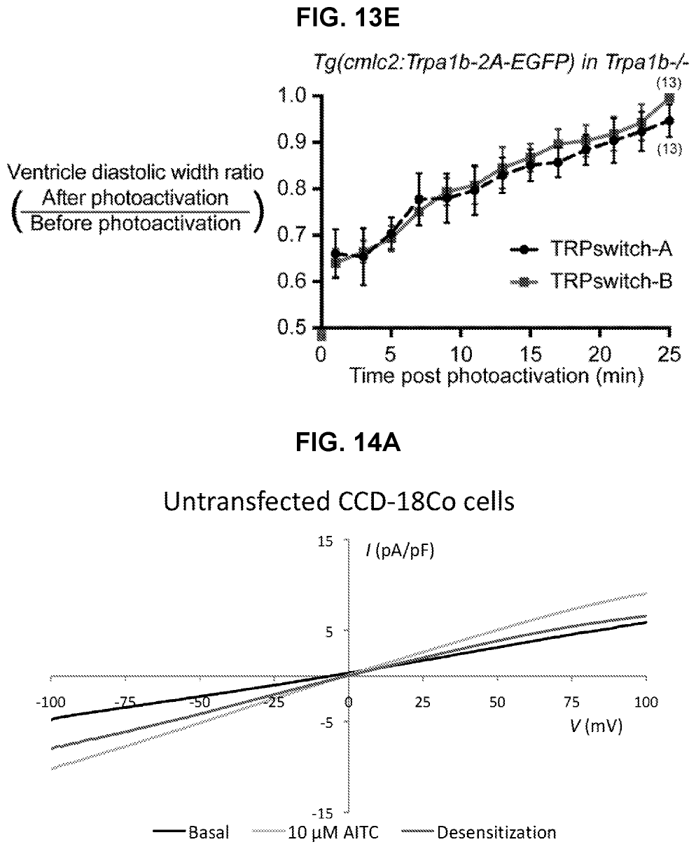

This analysis suggests that both TRPswitch-A and TRPswitch-B have thermal half-lives in the scale of hours in DMSO. However, the half-life of the biological response to these compounds was measured to be in the timescale of minutes during in vivo cardiac experiments (FIG. 13E). Several factors may account for the shorter half-life observed in vivo. Firstly, the thermal half-life of TRPswitches would be different under the conditions of the biological assays, for example in aqueous solution with only 1% DMSO. In fact, the thermal half-life of TRPswitches become shorter when measured in 30% water:DMSO (43 min and 1 hour for TRPswitch-A and —B, respectively). Secondly, although TRPA1/TRPswitch light-induced activity is reversible and repeatable, TRPswitch does not covalently bind to TRPA1 and is therefore free to diffuse away from the channel overtime. Thirdly, there might be biological adaptation during TRPA1 activation, such as receptor internalization, which leads to a shorter time of biological effect compared to the half-life measured by UV-Vis in vitro.

Most of the known TRPA1 ligands are electrophiles that activate TRPA1 via the covalent modification of cysteine residues present in the channel's cytoplasmic ankyrin repeat domain [48]. This includes the previously identified TRPA1 photoactivatable ligand, optovin [23-24]. Although both optovin and TRPswitch target TRPA1 and are activated by violet light, they are unique in their mechanism of action, reversibility, and kinetics. Unlike optovin and its derivatives, which act though light-induced covalent modification, TRPswitch's photoreversible TRPA1 activity depends on differential activity of its E and Z isomers. How isomerization triggers opening and closing of the TRPA1 channel remains unknown. Mechanistically, the fifth transmembrane (TM5) domain of TRPA1 has been shown to determine channel sensitivity to non-electrophilic agonists such as menthol and anethole [49-50]. Considering that menthol and anethole are structurally distinct molecules, TRPA1's TM5 may act as a general binding site for non-electrophilic agonists and may therefore be a candidate site for the binding of TRPswitch. Alternatively, a peptidergic scorpion toxin (WaTx) was recently discovered that activates TRPA1 by binding to the same allosteric nexus that is covalently modified by electrophilic irritants [51]. It is possible that the TRPswitches bind to this allosteric nexus on TRPA1 channel as well.

The development of PCLs for endogenous targets offers theoretical advantages in clinical applications since the introduction of exogenous gene products is not needed. The common approach for PCL discovery is to modify a known biologically active molecule with photoswitchable functionality, such as “azologization” [52]. Although this approach has had some success [11-15], it is limited by the need for already identified active molecules for specific targets, which have a defined mechanism of activation and that contain a chemical structure amenable to incorporation of a photoswitch. The screening strategy described here offers an alternative, phenotypic approach for the discovery of photoswitchable ligands of novel endogenous targets [53]. Since screening is performed using intact animals, hit compounds identified are, by definition, biologically active and likely to have minimal general toxicity. It should also be noted that despite having excellent and tunable switching properties, heteroaryl azo motifs are still significantly under-explored compared to their azobenzene counterparts in photoaddressible applications including as PCLs [54]. The discovery that the azopyrazole-containing TRPswitch-B exhibits a longer half-life and more efficient photoswitching than the azobenzene-containing TRPswitch-A is a good indication that heteroaryl azo photoswitches are good alternatives for photoswitch optimization. Indeed, this is the first application of the high performance arylazopyrazoles [32, 40] for in vivo photopharmacology, showcasing the potential of this scaffold for future studies.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All the various embodiments, aspects, and options disclosed herein can be combined in any variations or iterations. The scope of the compositions, formulations, methods, and processes described herein include all actual or potential combinations of embodiments, aspects, options, examples, and preferences herein described. The compositions, formulations, or methods described herein may omit any component or step, substitute any component or step disclosed herein, or include any component or step disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the specification discloses and describes merely exemplary embodiments. All patents and publications cited herein are incorporated by reference herein for the specific teachings thereof.

### REFERENCES

1. Sugano et al., “Immune responses to adeno-associated virus type 2 encoding channelrhodopsin-2 in a genetically blind rat model for gene therapy.” Gene Ther. 18(3): 266-174 (2011).
2. Li et al., “Electrophysiological Properties and Viability of Neonatal Rat Ventricular Myocyte Cultures with Inducible ChR2 Expression.” Sci. Rep. 7(1): 1531 (2017).
3. Tochitsky et al., “Restoring visual function to the blind retina with a potent, safe and long-lasting photoswitch.” Sci. Rep. 7: 45487 (2017).
4. Tochitsky et al., “How Azobenzene Photoswitches Restore Visual Responses to the Blind Retina.” Neuron 92(1): 100-113 (2016).
5. Mourot et al., “Tuning photochromic ion channel blockers.” ACS Chem. Neurosci. 2(9): 536-543 (2011).
6. Berndt et al., “Bi-stable neural state switches.” Nat. Neurosci. 12 (2): 229-234 (2009).
7. Yizhar et al., “Neocortical excitation/inhibition balance in information processing and social dysfunction.” Nature 477(7363): 171-178 (2011).
8. Bamann et al., “Structural guidance of the photocycle of channelrhodopsin-2 by an interhelical hydrogen bond.” Biochemistry 49(2): 267-278 (2010).
9. Guru et al., “Making Sense of Optogenetics.”Int. J. Neuropsychopharmacol. 18(11): pyv079 (2015).
10. Yizhar et al., “Optogenetics in neural systems.” Neuron 71(1): 9-34 (2011).
11. Frank et al., “Photoswitchable fatty acids enable optical control of TRPV1.” Nat Commun. 6: 7118 (2015).
12. Stein et al., “Optical control of TRPV1 channels.” Angew Chem. Int. Ed. Engl. 52(37): 9845-9848 (2013).
13. Schonberger et al., “Controlling epithelial sodium channels with light using photoswitchable amilorides.” Nat. Chem. 6(8): 712-719 (2014).
14. Banghart et al., “Photochromic blockers of voltage-gated potassium channels.” Angew Chem. Int. Ed. Engl. 48(48): 9097-9101 (2009).
15. Mourot et al., “Rapid optical control of nociception with an ion-channel photoswitch.” Nat. Methods 9(4): 396-402 (2012).
16. Laprell et al., “Optical control of NMDA receptors with a diffusible photoswitch.” Nat. Commun. 6: 8076 (2015).
17. Thapaliya et al., “Locked-Azobenzene: Testing the Scope of a Unique Photoswitchable Scaffold for Cell Physiology.” ACS Chem. Neurosci. 10(5): 2481-2488 (2019).
18. Izquierdo-Serra et al., “Optical control of endogenous receptors and cellular excitability using targeted covalent photoswitches.” Nat. Commun. 7: 12221 (2016).
19. Cabre et al., “Synthetic Photoswitchable Neurotransmitters Based on Bridged Azobenzenes.” Org. Lett. 21(10): 3780-3784 (2019).
20. Chen and Hackos, “TRPA1 as a drug target—promise and challenges.” Naunyn Schmiedebergs Arch. Pharmacol. 388 (4): 451-463 (2015).
21. Nassini et al., “The TRPA1 channel in inflammatory and neuropathic pain and migraine.” Rev. Physiol. Biochem. Pharmacol. 167: 1-43 (2014).
22. Meseguer et al., “TRPA1 channels mediate acute neurogenic inflammation and pain produced by bacterial endotoxins.” Nat. Commun. 5: 3125 (2014).
23. Kokel et al., “Photochemical activation of TRPA1 channels in neurons and animals.” Nat. Chem. Biol. 9(4): 257-263 (2013).
24. Lam et al., “A high-conductance chemo-optogenetic system based on the vertebrate channel Trpa1b.” Sci. Rep. 7(1): 11839 (2017).
25. Nagata et al., “Nociceptor and hair cell transducer properties of TRPA1, a channel for pain and hearing.” J. Neurosci. 25(16): 4052-4061 (2005).
26. Klan et al., “Photoremovable protecting groups in chemistry and biology: reaction mechanisms and efficacy.” Chem. Rev. 113(1): 119-191 (2013).
27. van Dijken et al., “Acylhydrazones as Widely Tunable Photoswitches.” J. Am. Chem. Soc. 137(47): 14982-14991 (2015).
28. Bandara and Burdette, “Photoisomerization in different classes of azobenzene.” Chem. Soc. Rev. 41(5): 1809-1825 (2012).
29. Beharry and Woolley, “Azobenzene photoswitches for biomolecules.” Chem. Soc. Rev. 40(8): 4422-4437 (2011).
30. Dong et al., “Red-Shifting Azobenzene Photoswitches for In Vivo Use.” Acc. Chem. Res. 48(10): 2662-2670 (2015).
31. Szymanski et al., “Reversible photocontrol of biological systems by the incorporation of molecular photoswitches.” Chem. Rev. 113(8): 6114-6178 (2013).
32. Calbo et al., “Tuning Azoheteroarene Photoswitch Performance through Heteroaryl Design.” J. Am. Chem. Soc. 139(3): 1261-1274 (2017).
33. Crespi et al., “Unraveling the Thermal Isomerization Mechanisms of Heteroaryl Azoswitches: Phenylazoindoles as Case Study.” J. Phys. Chem. A 123(9): 1814-1823 (2019).
34. Waldeck, “Photoisomerization Dynamics of Stilbenes.” Chem. Rev. 91(3): 415-436 (1991).
35. Prober et al., “Zebrafish TRPA1 channels are required for chemosensation but not for thermosensation or mechanosensory hair cell function.” J. Neurosci. 28(40): 10102-10110 (2008).
36. Douglass et al., “Escape behavior elicited by single, channelrhodopsin-2-evoked spikes in zebrafish somatosensory neurons.” Curr. Biol. 18(15): 1133-1137 (2008).
37. Garcia-Amoros and Velasco, “Recent advances towards azobenzene-based light-driven real-time information-transmitting materials.” Beilstein J. Org. Chem. 8: 1003-1017 (2012).
38. Bandara and Burdette, “Photoisomerization in different classes of azobenzene.” Chem. Soc. Rev. 41(5): 1809-1825 (2012).
39. Weston et al., “Arylazopyrazoles: azoheteroarene photoswitches offering quantitative isomerization and long thermal half-lives.” J. Am. Chem. Soc. 136(34): 11878-11881 (2014).
40. Calbo et al., “A combinatorial approach to improving the performance of azoarene photoswitches.” Beilstein J. Org. Chem. 15: 2753-2764 (2019).
41. Weston et al., “Toward Photopharmacological Antimicrobial Chemotherapy Using Photoswitchable Amidohydrolase Inhibitors.” ACS Infect. Dis. 3(2): 152-161 (2017).
42. Hinman et al., “TRP channel activation by reversible covalent modification.” Proc. Natl. Acad. Sci. USA 103(51): 19564-19568 (2006).
43. Yang et al., “TRPA1-expressing lamina propria mesenchymal cells regulate colonic motility.” JCI Insight 4(9) (2019).
44. Riefolo et al., “Optical Control of Cardiac Function with a Photoswitchable Muscarinic Agonist.” J. Am. Chem. Soc. 141(18): 7628-7636 (2019).
45. Koivisto et al, “TRPA1 Antagonists for Pain Relief.” Pharmaceuticals (Basel)11(4):117 (2018).
46. Cabre et al., Rationally designed azobenzene photoswitches for efficient two-photon neuronal excitation.” Nat. Commun. 10(1): 907 (2019).
47. Dong et al., Near-Infrared Photoswitching of Azobenzenes under Physiological Conditions.” J. Am. Chem. Soc. 139(38): 13483-13486 (2017).
48. Macpherson et al., Noxious compounds activate TRPA1 ion channels through covalent modification of cysteines.” Nature 445(7127): 541-545 (2007).
49. Xiao et al., “Identification of transmembrane domain 5 as a critical molecular determinant of menthol sensitivity in mammalian TRPA1 channels.” J. Neurosci. 28(39): 9640-9651 (2008).
50. Memon et al., “trans-Anethole of Fennel Oil is a Selective and Nonelectrophilic Agonist of the TRPA1 Ion Channel.” Mol. Pharmacol. 95(4): 433-441 (2019).
51. Lin King et al., A Cell-Penetrating Scorpion Toxin Enables Mode-Specific Modulation of TRPA1 and Pain.” Cell 178(6): 1362-1374 e16 (2019).
52. Morstein et al., “Mapping the Azolog Space Enables the Optical Control of New Biological Targets.” ACS Cent. Sci. 5(4): 607-618 (2019).
53. Fuchter, “On the promise of photopharmacology using photoswitches: a medicinal chemist's perspective.” J. Med. Chem. 10.1021/acs.jmedchem.0c00629 (2020).
54. Crespi et al., “Heteroaryl azo dyes as molecular photoswitches.” Nat. Rev. Chem. 3(3): 133-146 (2019).
55. SHELXTL v5.1, Bruker AXS, Madison, Wis., 1998.
56. Sheldrick, “SHELX-2013.” Acta Cryst., C71, 3-8 (2015).
57. Spek “PLATON, A Multipurpose Crystallographic Tool, Utrecht University, Utrecht, The Netherlands (2003, 2009). See also Spek, Acta. Cryst. C71: 9-18 (2015).
58. Priewisch and Ruck-Braun, “Efficient Preparation of Nitrosoarenes for the Synthesis of Azobenzenes,” J. Org. Chem. 70(6): 2350-2352 (2005).

### EXAMPLES

### Example 1

### General Methods

All reagents and solvents were purchased from commercial sources and used as supplied unless otherwise indicated. All reactions were carried out under an inert atmosphere and using anhydrous solvents. All reactions were monitored by thin-layer chromatography (TLC) using Merck silica gel 60 F254 plates (0.25 mm). TLC plates were visualised using UV light (254 nm) and/or by using the appropriate TLC stain. Silica column chromatography was performed using Merck Silica Gel 60 (230-400 mesh) treated with a solvent system specified in the individual procedures. Solvents were removed by rotary evaporator at 40° C. or below and the compounds further dried using high vacuum pumps. Infrared spectra were recorded neat on an Agilent Cary 630 FTIR. Reported absorptions are in wavenumbers (cm⁻¹). ¹H and ¹³C NMR were recorded on a Bruker Avance 400 spectrometer at 400 MHz and 100 MHz, respectively. Chemical shifts (6) are quoted in ppm (parts per million) downfield from tetramethylsilane, referenced to residual solvent signals: ¹H δ=7.26 (CHCl₃), 2.50 (DMSO-d₅), ¹³C δ=77.16 (CDCl₃), 39.52 (DMSO-d₆). The following abbreviations are used to designate multiplicity within ¹H NMR analysis; s=singlet, d=doublet, t=triplet, q=quartet, m=multiplet, br.=broad signal. High-resolution mass spectra (ESI, APCI) were recorded by the Imperial College London Department of Chemistry Mass Spectroscopy Service using a Micromass Autospec Premier and Micromass LCT Premier spectrometer. LCMS analysis of compounds were conducted using a reverse phase LCMS Waters 2767 system equipped with a photodiode array and an ESI mass spectrometer using an XBridge C18 (5 μm, 100 mm×4.6 mm) column, equipped with an XBridge C18 guard column (5 μm, 4.6 mm×20 mm). An eluent of MeCN and H₂O was used: 0-10 min 50-98% MeCN, 10-12 min 98% MeCN, 12-13 min 98 to 50% MeCN, 13-17 min 50% MeCN (Method A). Flow rate: 1.2 mL/min. Alternatively, a PerkinElmer Series 200 HPLC equipped with a Supelco LC-SI (5 μm, 300 mm×4.0 mm) column was used. An eluent of isopropanol and hexane was used: 0-15 min 90% IPA (Method B) or 0-30 min 90% IPA (Method C). Flow rate: 0.3 mL/min.

### Synthetic Route and Procedures

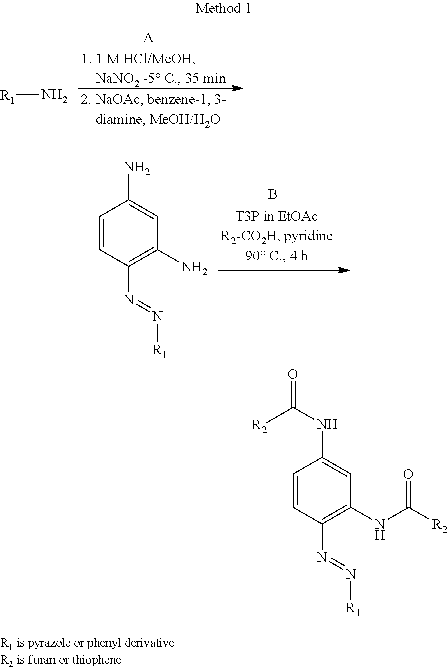

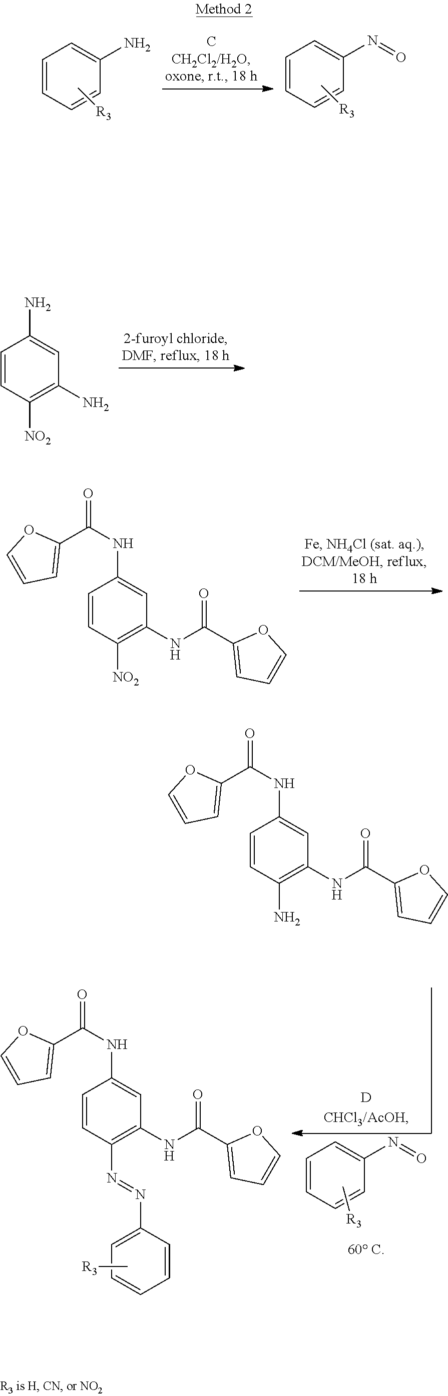

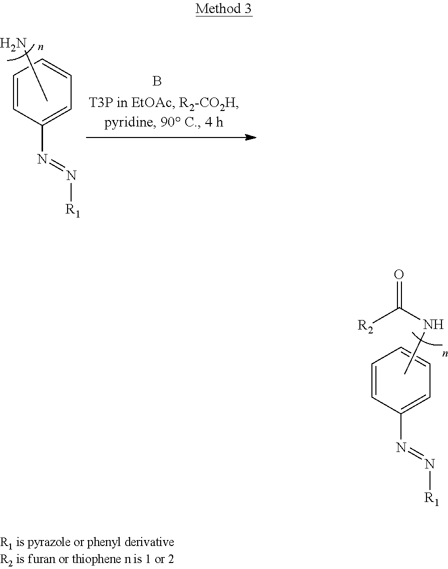

### General Procedure A

A suspension of the relevant amine/aniline (1.0 eq.) in 1 M HCl (4 mL/mmol) was prepared and, if necessary, MeOH was added to aid solubility. This solution was cooled to −5° C. using an ice-brine bath before NaNO₂ (1.1 eq.) in H₂O (1 mL/mmol) was added dropwise. The resulting solution was stirred for 35 min before a solution of benzene-1,3-diamine (1.0 eq.) and NaOAc (3 eq.) in MeOH/H₂O was added gradually. The pH of the resulting suspension was checked and, if necessary, made to pH 9-10 using 2 M NaOH. EtOAc was added, separated off and an extraction carried out with EtOAc three more times. The combined organic phases were washed with brine, dried over anhydrous Mg₂SO₄, concentrated in vacuo, and subjected to purification by silica column chromatography using solvent systems as described in the individual protocols.

### General Procedure B

To a solution of the relevant aniline (1.0 eq.) and acid (2.0 eq.) in pyridine (5 mL/mmol) propylphosphonic anhydride (T3P) 50 wt. % in EtOAc (3.0 eq.) was added. The reaction was heated to 90° C. for 4 hr. EtOAc/H₂O were added, and the two layers separated. The aqueous layer was extracted with EtOAc three more times. The combined organic phases were washed with 1 M HCl, saturated NaHCO₃, brine, dried over anhydrous Mg₂SO₄, concentrated in vacuo, and subjected to purification by silica column chromatography using solvent systems as described in the individual protocols.

### General Procedure C

The synthesis of nitrosobenzene derivatives followed a literature procedure described by Priewisch and RQck-Braun, J. Org. Chem. 70(6): 2350-2352 (2005) [58].

### General Procedure D

To a solution of N,N′-(4-amino-1,3-phenylene)bis(furan-2-carboxamide) (1.0 eq.) in AcOH:CHCl₃ (1:1) was added the relevant nitrosobenzene derivative (1.2 eq.). The reaction was heated to 60° C. and monitored by thin layer chromatography. Upon completion of the reaction, the mixture was cooled room temperature, EtOAc/H₂O was added, and the two layers separated. The aqueous layer was extracted with EtOAc three additional times. The combined organic phases were washed with 1 M HCl, saturated NaHCO₃, brine, dried over anhydrous Mg₂SO₄, concentrated in vacuo, and subjected to purification by silica column chromatography.

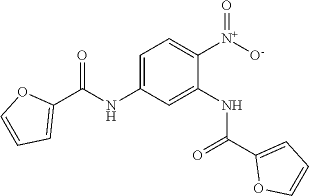 [SMILES: O=C(Nc1ccc([N+](=O)[O-])c(NC(=O)c2ccco2)c1)c1ccco1]

### N,N′-(4-amino-1,3-phenylene)bis(furan-2-carboxamide)

4-nitrobenzene-1,3-diamine (1.5 g, 9.80 mmol) was added to neat 2-furoyl chloride (50 mL). The reaction was then heated to 100° C. and DMF (10 mL) was added in one portion. The reaction was held at this temperature for 18 h. Subsequently, the reaction was cooled and poured into ice-water to precipitate the crude product. This was further purified by recrystallisation from CHCl₃/MeOH. The title compound was obtained as a yellow solid (2.3 g, 6.68 mmol, 68% yield). ¹H NMR (400 MHz, DMSO-d₆) δ 11.09 (s, 1H), 10.79 (s, 1H), 8.77 (d, J=2.4 Hz, 1H), 8.17 (d, J=9.3 Hz, 1H), 8.00 (dd, J=13.0, 2.1 Hz, 2H), 7.77 (dd, J=9.3, 2.4 Hz, 1H), 7.46 (d, J=3.8 Hz, 1H), 7.37 (d, J=3.9 Hz, 1H), 6.75 (ddd, J=9.0, 3.4, 1.7 Hz, 2H); ¹³C NMR (101 MHz, DMSO-d₆) δ 156.7, 155.9, 146.9, 146.8, 146.7, 144.8, 134.8, 133.7, 126.9, 116.4, 116.3, 115.7, 113.6, 112.9, 112.5; HRMS m/z calculated for C₁₆H₁₂N₃O₆ [M+H]⁺ 342.0726; found 342.0726.

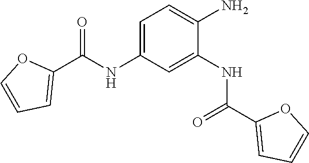 [SMILES: Nc1ccc(NC(=O)c2ccco2)cc1NC(=O)c1ccco1]

### N,N′-(4-amino-1,3-phenylene)bis(furan-2-carboxamide)

To a suspension of N,N′-(4-nitro-1,3-phenylene)bis(furan-2-carboxamide) (1.0 g, 2.93 mmol) in DCM:MeOH (2:1, 50 mL) was added saturated aqueous NH₄Cl (1.5 mL) and Fe (8.5 g, 151 mmol). The reaction was refluxed for 18 h. Subsequently, the mixture was filtered through Celite, concentrated in vacuo and the crude material purified by passing it through a silica plug. The title compound was obtained as a brown solid (800 mg, 2.57 mmol, 88% yield). ¹H NMR (400 MHz, CDCl₃) δ 8.42 (s, 1H), 8.31 (s, 1H), 7.65 (d, J=2.4 Hz, 1H), 7.42 (ddd, J=7.5, 1.8, 0.9 Hz, 2H), 7.27 (dd, J=8.8, 2.6 Hz, 1H), 7.19-7.09 (m, 2H), 6.68 (d, J=8.6 Hz, 1H), 6.46 (dt, J=3.6, 1.8 Hz, 2H), 3.47 (s br., 2H); ¹³C NMR (101 MHz, CDCl₃) δ 156.8, 156.4, 147.9, 147.5, 144.7, 144.3, 137.6, 129.4, 124.1, 119.9, 118.5, 117.8, 115.4, 114.8, 112.4; HRMS m/z calculated for C16H14N3O4 [M+H]⁺ 312.0984; found 312.0979.

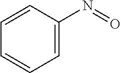

### Nitrosobenzene

Synthesized from aniline according to General Procedure C. The crude product was used immediately in the next step without further purification due to its perceived instability.

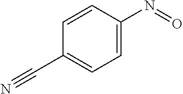

### 4-nitrosobenzonitrile

Synthesized from 4-aminobenzonitrile according to General Procedure C. The crude product was used immediately in the next step without further purification due to its perceived instability.

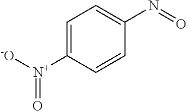

### 1-nitro-4-nitrosobenzene

Synthesized from 4-nitroaniline according to General Procedure C. The crude product was used immediately in the next step without further purification due to its perceived instability.

### Compound Synthesis

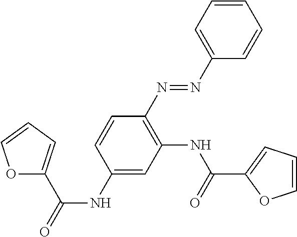 [SMILES: O=C(Nc1ccc(/N=N/c2ccccc2)c(NC(=O)c2ccco2)c1)c1ccco1]

### (E)-N,N′-(4-(phenyldiazenyl)-1,3-phenylene)bis(furan-2-carboxamide) (Compound 1)

General procedure B was followed using (E)-4-(phenyldiazenyl)benzene-1,3-diamine hydrochloride (200 mg, 0.80 mmol), furan-2-carboxylic acid (180 mg, 1.61 mmol) and T3P (1.4 mL, 2.4 mmol). Purified using a gradient of 0-20% EtOAc/CH₂Cl₂. The title compound was obtained as an orange solid (186 mg, 0.46 mmol, 58% yield). LCMS Method A (9.19 min); ¹H NMR (400 MHz, DMSO-d₆) δ 11.49 (s, 1H), 10.66 (s, 1H), 9.00 (d, J=2.3 Hz, 1H), 8.11 (dd, J=1.8, 0.8 Hz, 1H), 8.02-7.82 (m, 5H), 7.69-7.52 (m, 3H), 7.48 (dd, J=3.6, 0.8 Hz, 1H), 7.39 (dd, J=3.5, 0.8 Hz, 1H), 6.77 (ddd, J=19.3, 3.5, 1.7 Hz, 2H); ¹³C NMR (101 MHz, DMSO-d₆) δ 156.5, 155.8, 151.9, 147.4, 147.1, 146.4, 146.3, 142.9, 135.8, 135.3, 131.3, 129.7, 123.6, 122.3, 115.7, 115.7, 115.5, 112.8, 112.3, 111.5; HRMS m/z calculated for C₂₂H₁₆N₄O₄Na [M+H]⁺ 423.1060; found 423.1069; IR: 3332, 3131, 1672, 1657.

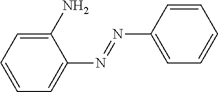 [SMILES: Nc1ccccc1/N=N/c1ccccc1]

### (E)-2-(phenyldiazenyl)aniline

To a stirred solution of 1,2-diaminobenzene (820 mg, 7.59 mmol) and AcOH (1.7 mL, 30 mmol) in CHCl₃ (20 mL) was added nitrosobenzene (812 mg, 7.59 mmol). The mixture was refluxed for 24 h. The reaction was then concentrated in vacuo, with any AcOH subjected to azeotropic removal with toluene. The crude was purified by silica column chromatography on a gradient of 5-7% EtOAc/pentane. The title compound was obtained as a maroon solid (608 mg, 3.08 mmol, 41% yield). LCMS Method A (6.79 min); ¹H NMR (400 MHz, CDCl₃) δ 7.89 (d, J=7.7 Hz, 2H), 7.61-7.42 (m, 2H), 7.32-7.20 (m, 1H), 6.87 (t, J=7.6 Hz, 1H), 6.80 (d, J=8.2 Hz, 1H), 5.93 (br. s, 2H); ¹³C NMR (101 MHz, CDCl₃) δ 153.1, 143.1, 137.2, 132.4, 130.1, 129.2, 127.8, 122.3, 117.5, 117.1; HRMS m/z calculated for C₁₂H₁₂N₃[M+H]⁺ 198.1031; found 198.1025; IR: 3463, 3364, 3058, 1603, 1570.

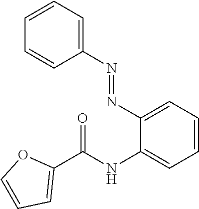 [SMILES: O=C(Nc1ccccc1/N=N/c1ccccc1)c1ccco1]

### (E)-N-(2-(phenyldiazenyl)phenyl)furan-2-carboxamide (Compound 2)

General procedure B was followed using (E)-2-(phenyldiazenyl)aniline (100 mg, 0.51 mmol), furan-2-carboxylic acid (114 mg, 1.02 mmol) and T3P (0.9 mL, 1.5 mmol). Purified using a gradient of 0-10% EtOAc/CH₂Cl₂. The title compound was obtained as a yellow solid (82 mg, 0.28 mmol, 55% yield). LCMS Method A (10.29 min); ¹H NMR (400 MHz, DMSO-d₆) δ 11.15 (s, 1H), 8.46 (dd, J=8.3, 1.3 Hz, 1H), 8.10-8.06 (m, 1H), 8.00-7.94 (m, 2H), 7.87 (dd, J=8.1, 1.5 Hz, 1H), 7.70-7.56 (m, 4H), 7.40-7.31 (m, 2H), 6.77 (dd, J=3.5, 1.8 Hz, 1H); 156.0, 151.9, 147.3, 146.4, 140.1, 135.0, 132.8, 131.9, 129.7, 124.5, 122.6, 121.6, 121.3, 115.7, 112.8; HRMS m/z calculated for C₁₇H₁₄N₃O₂ [M+H]⁺ 292.1086; found 292.1093; IR: 3147, 3101, 3060, 1660, 1577.

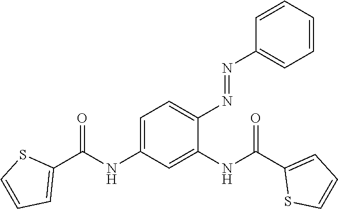 [SMILES: O=C(Nc1ccc(/N=N/c2ccccc2)c(NC(=O)c2cccs2)c1)c1cccs1]

### (E)-N,N′-(4-(phenyldiazenyl)-1,3-phenylene)bis(thiophene-2-carboxamide) (Compound 3)

General procedure B was followed using (E)-4-(phenyldiazenyl)benzene-1,3-diamine hydrochloride (500 mg, 2.01 mmol), thiophene-2-carboxylic acid (515 mg, 4.02 mmol) and T3P (3.6 mL, 6.0 mmol). Purified using a gradient of 0-5% EtOAc/CH₂Cl₂. The title compound was obtained as an orange solid (592 mg, 1.37 mmol, 68% yield). LCMS Method A (10.72 min); ¹H NMR (400 MHz, DMSO-d₆) δ 10.94 (s, 1H), 10.66 (s, 1H), 8.73 (d, J=2.0 Hz, 1H), 8.14 (dd, J=3.7, 1.2 Hz, 1H), 8.03 (dd, J=3.8, 1.2 Hz, 1H), 7.97-7.89 (m, 4H), 7.90-7.82 (m, 2H), 7.65-7.50 (m, 3H), 7.28 (ddd, J=14.8, 5.0, 3.8 Hz, 2H); ¹³C NMR (101 MHz, DMSO-d₆) δ 160.3, 159.8, 152.3, 142.8, 139.6, 139.4, 137.5, 136.4, 132.7, 132.5, 131.2, 129.9, 129.5, 128.5, 128.3, 122.5, 120.3, 116.3, 113.6; HRMS m/z calculated for C₂₂H₁₇N₄O₂S₂[M+H]⁺ 433.0793; found 433.0806; IR: 3387, 3100, 1664, 1641, 1592.

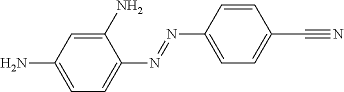 [SMILES: N#Cc1ccc(/N=N/c2ccc(N)cc2N)cc1]

### (E)-4-((2,4-diaminophenyl)diazenyl)benzonitrile

General procedure A was followed using 4-aminobenzonitrile (500 mg, 4.23 mmol), NaNO₂ (321 mg, 4.66 mmol), benzene-1,3-diamine (457 mg, 4.23 mmol) and NaOAc (1.04 g, 12.7 mmol). Purified using a gradient of 20-80% EtOAc/CH₂Cl₂. The title compound was obtained as a black solid (391 mg, 1.65 mmol, 39% yield). LCMS Method A (0.98 min); ¹H NMR (400 MHz, DMSO-d₆) δ 7.87-7.75 (m, 4H), 7.38 (d, J=8.9 Hz, 1H), 6.30 (br. s, 2H), 6.05 (dd, J=8.9, 2.3 Hz, 1H), 5.85 (d, J=2.1 Hz, 1H); 13C NMR (101 MHz, DMSO-d₆) δ 156.1, 154.7, 133.2, 130.1, 121.3, 119.4, 108.0, 107.1, 95.6; HRMS m/z calculated for C₁₃H₁₂N₅[M+H]⁺ 238.1088; found 238.1093; IR: 3454, 3425, 3357, 3233, 2214, 1577.

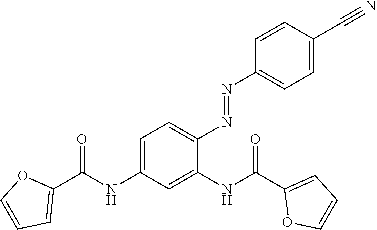 [SMILES: N#Cc1ccc(/N=N/c2ccc(NC(=O)c3ccco3)cc2NC(=O)c2ccco2)cc1]

### (E)-N,N′-(4-((4-cyanophenyl)diazenyl)-1,3-phenylene)bis(furan-2-carboxamide) (Compound 4)

General procedure B was followed using (E)-4-((2,4-diaminophenyl)diazenyl)benzonitrile (318 mg, 1.34 mmol), furan-2-carboxylic acid (301 mg, 2.68 mmol) and T3P (2.4 mL, 4.0 mmol). Purified using a gradient of 0-10% EtOAc/CH₂Cl₂. The title compound was obtained as a red solid (467 mg, 1.10 mmol, 82% yield). LCMS Method B (10.38 min); ¹H NMR (400 MHz, DMSO-d₆) δ 11.40 (s, 1H), 10.68 (s, 1H), 8.98 (d, J=2.2 Hz, 1H), 8.10-7.95 (m, 6H), 7.92-7.79 (m, 2H), 7.47 (dd, J=3.6, 0.8 Hz, 1H), 7.37 (dd, J=3.5, 0.8 Hz, 1H), 6.75 (ddd, J=15.2, 3.5, 1.7 Hz, 2H); ¹³C NMR (101 MHz, DMSO-d6) δ 156.5, 155.9, 154.1, 147.3, 147.1, 146.4, 144.1, 136.1, 136.0, 134.0, 123.8, 122.9, 118.6, 115.8, 115.8, 112.9, 112.8, 112.4, 111.4; HRMS m/z calculated for C₂₃H₁₆N₅O₄[M+H]⁺ 426.1202; found 426.1206; IR: 3133, 3112, 2223, 1653, 1579.

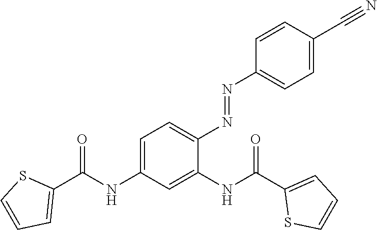 [SMILES: N#Cc1ccc(/N=N/c2ccc(NC(=O)c3cccs3)cc2NC(=O)c2cccs2)cc1]

### (E)-N,N′-(4-((4-cyanophenyl)diazenyl)-1,3-phenylene)bis(thiophene-2-carboxamide) (Compound 5)

General procedure B was followed using (E)-4-((2,4-diaminophenyl)diazenyl)benzonitrile (102 mg, 0.43 mmol), thiophene-2-carboxylic acid (110 mg, 0.86 mmol) and T3P (0.8 mL, 1.3 mmol). Purified using a gradient of 0-10% EtOAc/CH²Cl². The title compound was obtained as a red solid (151 mg, 0.33 mmol, 77% yield). LCMS Method B (9.85 min); ¹H NMR (400 MHz, DMSO-d₆) 1H NMR (400 MHz, DMSO-d₆) δ 10.88 (s, 1H), 10.71 (s, 1H), 8.74 (d, J=2.2 Hz, 1H), 8.14 (dd, J=3.8, 1.2 Hz, 1H), 8.11-8.00 (m, 5H), 7.93 (ddd, J=6.0, 4.9, 1.1 Hz, 2H), 7.90-7.82 (m, 2H), 7.28 (ddd, J=10.1, 5.0, 3.8 Hz, 2H); ¹³C NMR (101 MHz, DMSO-d₆) δ 160.3, 159.8, 154.4, 143.9, 139.5, 139.2, 137.6, 137.3, 133.8, 132.8, 132.6, 130.0, 129.7, 128.5, 128.2, 123.1, 120.2, 118.5, 116.2, 113.4, 112.7; HRMS m/z calculated for C₂₃H₁₄N₅O₂S₂[M−H]⁻ 456.0589; found 456.0593; IR: 3286, 3100, 2223, 1646, 1580.

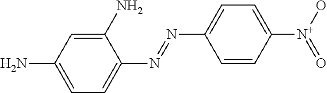

### (E)-4-((4-nitrophenyl)diazenyl)benzene-1,3-diamine

General procedure A was followed using 4-nitroaniline (500 mg, 3.62 mmol), NaNO₂ (275 mg, 3.98 mmol), benzene-1,3-diamine (391 mg, 3.62 mmol) and NaOAc (891 mg, 10.9 mmol). Purified using a gradient of 20-80% EtOAc/CH₂Cl₂. The title compound was obtained as a black solid (289 mg, 1.12 mmol, 31% yield). LCMS Method A (1.03 min); ¹H NMR (400 MHz, DMSO-d₆) δ 8.23 (dd, J=9.2, 5.2 Hz, 2H), 7.81 (dd, J=9.0, 5.4 Hz, 2H), 7.40 (d, J=9.0 Hz, 1H), 6.44 (br. s, 2H), 6.10 (d, J=9.0 Hz, 1H), 5.87 (d, J=2.2 Hz, 1H); ¹³C NMR (101 MHz, DMSO-d₆) δ 157.9, 155.2, 144.7, 130.8, 124.9, 121.0, 107.9, 95.3; HRMS m/z calculated for C₁₂H₁₂N₅O₂ [M+H]⁺ 258.0991; found 258.1000; IR: 3480, 3373, 3236, 1636, 1617.

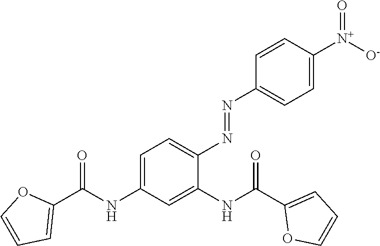 [SMILES: O=C(Nc1ccc(/N=N/c2ccc([N+](=O)[O-])cc2)c(NC(=O)c2ccco2)c1)c1ccco1]

### (E)-N,N′-(4-((4-nitrophenyl)diazenyl)-1,3-phenylene)bis(furan-2-carboxamide) (Compound 6)

General procedure B was followed using (E)-4-((4-nitrophenyl)diazenyl)benzene-1,3-diamine (161 mg, 0.63 mmol), furan-2-carboxylic acid (140 mg, 1.25 mmol) and T3P (1.1 mL, 1.9 mmol). Purified using a gradient of 0-20% EtOAc/CH₂Cl₂. The title compound was obtained as a red solid (203 mg, 0.46 mmol, 73% yield). ¹H NMR (400 MHz, DMSO-d₆) δ 11.54 (s, 1H), 10.76 (s, 1H), 9.05 (d, J=2.3 Hz, 1H), 8.50 (d, J=9.1 Hz, 2H), 8.18-8.12 (m, 3H), 8.02-7.96 (m, 2H), 7.89 (dd, J=9.0, 2.2 Hz, 1H), 7.50 (dd, J=3.5, 0.8 Hz, 1H), 7.41 (dd, J=3.5, 0.8 Hz, 1H), 6.78 (ddd, J=20.2, 3.5, 1.7 Hz, 2H); ¹³C NMR (101 MHz, DMSO-d₆) δ 156.5, 155.9, 155.3, 148.1, 147.2, 147.0, 146.6, 146.5, 144.4, 136.2, 136.0, 125.3, 124.4, 123.2, 115.9, 115.8, 112.8, 112.3, 111.3; HRMS m/z calculated for C₂₂H₁₄N₅O₆ [M−H]⁻ 444.0944; found 444.0942; IR: 3412, 3357, 3114, 1684, 1581, 1506.

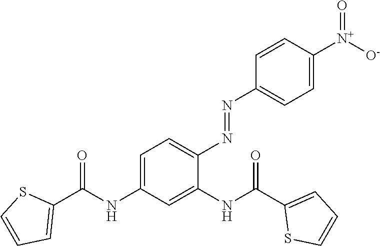 [SMILES: O=C(Nc1ccc(/N=N/c2ccc([N+](=O)[O-])cc2)c(NC(=O)c2cccs2)c1)c1cccs1]

### (E)-N,N′-(4-((4-nitrophenyl)diazenyl)-1,3-phenylene)bis(thiophene-2-carboxamide) (Compound 7)

General procedure B was followed using (E)-4-((4-nitrophenyl)diazenyl)benzene-1,3-diamine (96 mg, 0.37 mmol), thiophene-2-carboxylic acid (95 mg, 0.74 mmol) and T3P (0.7 mL, 1.2 mmol). Purified using a gradient of 0-20% EtOAc/CH₂Cl₂. The title compound was obtained as a maroon solid (114 mg, 0.24 mmol, 64% yield). ¹H NMR (400 MHz, DMSO-d₆) δ 10.91 (s, 1H), 10.73 (s, 1H), 8.77 (d, J=2.2 Hz, 1H), 8.45 (dd, J=9.0, 5.0 Hz, 2H), 8.18-8.09 (m, 3H), 8.04 (dd, J=3.8, 1.2 Hz, 1H), 7.98-7.82 (m, 4H), 7.29 (ddd, J=12.5, 4.9, 3.7 Hz, 2H); ¹³C NMR (101 MHz, DMSO-d₆) δ 160.4, 159.9, 155.7, 148.1, 144.2, 139.5, 139.2, 137.8, 137.6, 132.9, 132.7, 130.1, 129.9, 128.6, 128.3, 125.2, 123.4, 120.2, 116.3, 113.4; HRMS m/z calculated for C₂₂H₁₄N₅O₄S₂[M−H]⁻ 476.0487; found 476.0485; IR: 3392, 3097, 1670, 1640, 1508.

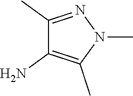

### 1,3,5-trimethyl-1H-pyrazol-4-amine

Synthesised according to literature precedent1 from 3,5-dimethyl-4-nitro-1H-pyrazole. ¹H NMR (400 MHz, CDCl₃) δ 3.64 (s, 3H), 2.45 (br. s, 2H), 2.13 (s, 3H), 2.11 (s, 3H); ¹³C NMR (101 MHz, CDCl₃) δ 138.8, 128.1, 122.8, 36.1, 10.8, 8.9; HRMS m/z calculated for C₆H₁₂N₃[M+H]⁺ 126.1026; found 126.1028.

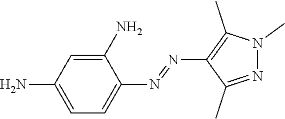 [SMILES: Cc1nn(C)c(C)c1/N=N/c1ccc(N)cc1N]

### (E)-4-((1,3,5-trimethyl-1H-pyrazol-4-yl)diazenyl)benzene-1,3-diamine

General procedure A was followed using 1,3,5-trimethyl-1H-pyrazol-4-amine (1.56 g, 12.5 mmol), NaNO₂ (947 mg, 13.7 mmol), benzene-1,3-diamine (1.35 g, 12.5 mmol) and NaOAc (3.07 g, 37.4 mmol). Purified using a gradient of 80-87% EtOAc/pentane (0.2% Et₃N). The title compound was obtained as a reddish-brown solid (1.38 g, 5.66 mmol, 45% yield). LCMS Method A (1.17 min), ¹H NMR (400 MHz, DMSO-d₆) δ 7.27 (d, J=8.7 Hz, 1H), 6.23 (br. s, 2H), 5.93 (dd, J=8.5, 2.3 Hz, 1H), 5.89 (d, J=2.3 Hz, 1H), 5.51 (br. s, 2H), 3.68 (s, 3H), 2.43 (s, 3H), 2.28 (s, 3H); ¹³C NMR (101 MHz, DMSO-d₆) δ 151.5, 145.8, 138.9, 134.7, 134.2, 129.9, 125.0, 104.6, 97.9, 35.7, 13.7, 9.5; HRMS m/z calculated for C₁₂H₁₇N₆ [M+H]⁺ 245.1515; found 245.1512; IR: 3421, 3430, 3314, 3379, 3198, 2918, 1617, 1497.

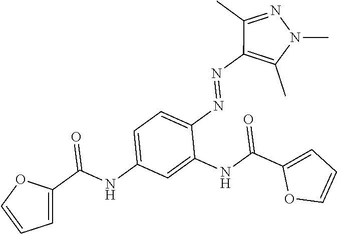 [SMILES: Cc1nn(C)c(C)c1/N=N/c1ccc(NC(=O)c2ccco2)cc1NC(=O)c1ccco1]

### (E)-N,N′-(4-((1,3,5-trimethyl-1H-pyrazol-4-yl)diazenyl)-1,3-phenylene)bis(furan-2-carboxamide) (Compound 8)

General procedure B was followed using (E)-4-((1,3,5-trimethyl-1H-pyrazol-4-yl)diazenyl)benzene-1,3-diamine (100 mg, 0.41 mmol), furan-2-carboxylic acid (91 mg, 0.82 mmol) and T3P (0.7 mL, 1.2 mmol). Purified using a gradient of 0-2% MeOH/CH₂Cl₂. The title compound was obtained as a yellow solid (109 mg, 0.25 mmol, 62% yield). LCMS Method C (15.49 min)¹H NMR (400 MHz, DMSO-d₆) δ 10.50 (s, 1H), 10.20 (s, 1H), 8.92 (d, J=2.1 Hz, 1H), 8.00-7.90 (m, 2H), 7.79-7.68 (m, 2H), 7.43 (dd, J=3.5, 0.9 Hz, 1H), 7.34 (d, J=3.5 Hz, 1H), 6.74 (ddd, J=11.6, 3.5, 1.7 Hz, 2H), 3.76 (s, 3H), 2.60 (s, 3H), 2.48 (s, 3H); ¹³C NMR (101 MHz, DMSO-d₆) δ 156.3, 155.2, 147.3, 146.1, 145.9, 140.7, 140.3, 140.1, 137.3, 135.3, 135.0, 116.0, 115.4, 115.1, 112.8, 112.2, 111.6, 69.8, 36.1, 14.2, 9.5; HRMS m/z calculated for C₂₂H₂₁N₆O₄ [M+H]⁺ 433.1621; found 433.1624; IR: 3421, 3340, 3307, 1680, 1647

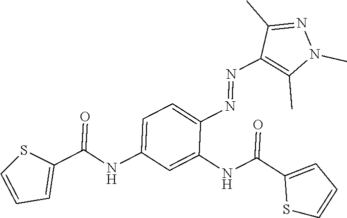 [SMILES: Cc1nn(C)c(C)c1/N=N/c1ccc(NC(=O)c2cccs2)cc1NC(=O)c1cccs1]

### (E)-N,N′-(4-((1,3,5-trimethyl-1H-pyrazol-4-yl)diazenyl)-1,3-phenylene)bis(thiophene-2-carboxamide) (Compound 9)

General procedure B was followed using (E)-4-((1,3,5-trimethyl-1H-pyrazol-4-yl)diazenyl)benzene-1,3-diamine (110 mg, 0.45 mmol), thiophene-2-carboxylic acid (115 mg, 0.90 mmol) and T3P (0.8 mL, 1.4 mmol). Purified using a gradient of 0-2% MeOH/CH₂Cl₂. The title compound was obtained as a yellow solid (150 mg, 0.32 mmol, 71% yield). ¹H NMR (400 MHz, DMSO-d₆) δ 10.52 (s, 1H), 10.16 (s, 1H), 8.52 (t, J=1.3 Hz, 1H), 8.11 (dd, J=3.8, 1.2 Hz, 1H), 7.97 (dd, J=3.7, 1.2 Hz, 1H), 7.90 (dt, J=5.1, 1.0 Hz, 2H), 7.75 (d, J=1.3 Hz, 2H), 7.25 (ddd, J=5.0, 3.8, 2.8 Hz, 2H), 3.74 (s, 3H), 2.55 (s, 3H), 2.35 (s, 3H); ¹³C NMR (101 MHz, DMSO-d₆) δ 160.0, 159.5, 140.5, 140.3, 139.8, 139.7, 139.7, 139.6, 135.4, 134.9, 132.3, 132.1, 129.5, 129.1, 128.3, 128.2, 116.8, 115.9, 114.7, 36.0, 14.0, 9.6; HRMS m/z calculated for C₂₂H₂₁N₆O₂S₂ [M+H]⁺ 465.1160; found 465.1167; IR: 3358, 3248, 1653, 1597.

### X-Ray Crystallography of TRPswitch-B (Compound 9)

Crystallography data were obtained for TRPswitch-B: C₂₂H₂₀N₆O₂S₂.CH₄O, M=496.60, orthorhombic, P2₁2₁2₁ (no. 19), a=6.3624(3), b=14.1164(8), c=27.4354(13) Å, V=2464.1(2) Å³, Z=4, Dc=1.339 g·cm⁻³, μ(Cu-Kα)=2.268 mm⁻¹, T=173 K, yellow plates, Agilent Xcalibur PX Ultra A diffractometer; 4089 independent measured reflections (Rᵢₙₜ=0.0588), F² refinement [55, 56] R1(obs)=0.0534, wR²₍ₐₗₗ₎=0.1334, 3286 independent observed absorption-corrected reflections [|Fₒ|>4σ(|Fₒ|), completeness to θ_{full} (67.7°)=98.9%], 300 parameters. The absolute structure of TRPswitch-B was determined by use of the Flack parameter [x=0.01(3)]. CCDC 1962764.

The included solvent was found to be highly disordered, and the best approach to handling this diffuse electron density was found to be the SQUEEZE routine of PLATON [57]. This suggested a total of 90 electrons per unit cell, equivalent to 22.5 electrons per asymmetric unit. Before the use of SQUEEZE, the solvent most resembled methanol (CH₄O, 18 electrons), and one methanol molecule corresponds to 18 electrons, so this was used as the solvent present. As a result, the atom list for the asymmetric unit is low by CH₄O (and that for the unit cell low by C₄H₁₆O₄) compared to what is actually presumed to be present.

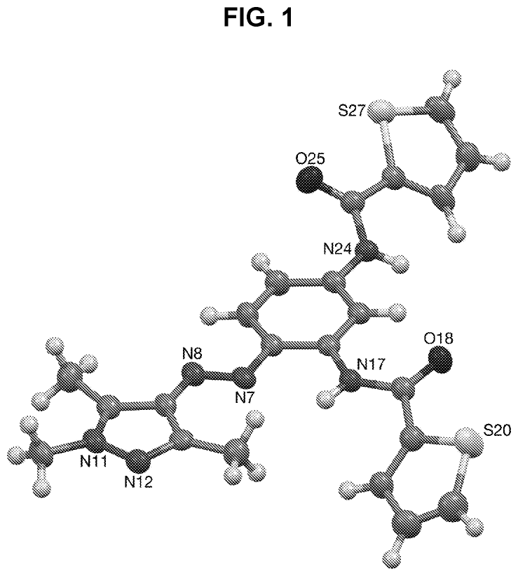

The N—H hydrogen atoms on N17 and N24 were located from ΔF maps and refined freely subject to an N—H distance constraint of 0.90 Å. The structure is shown in FIG. 1.

### Example 2

### Experimental Methods

### Thermal Isomerisation Kinetics

In order to determine the Z→E thermal isomerisation kinetics in DMSO-d₆ a ˜20 mM solution of the relevant compound was irradiated using a Luzchem LZC-4V photoreactor fitted with 12×8 W LZC-420 lamps (420 nm peak emission) until no further change was observable by ¹H NMR. For 30% water:DMSO samples, a 15 μM solution of the relevant compound was irradiated in a quartz cuvette using 365 nm LEDs (3×800 mW Nichia NCSU276A) until no further change was observed by UV-Vis spectroscopy. Compounds (˜25 μM solutions in DMSO) were treated analogously. In the case of compounds studied by NMR, the change in Z-isomer percentage was followed as a function of time through integration of the relevant peaks. In the case of compounds studied by UV-Vis, absorption spectra were collected over time while the sample was held at the stated temperature.

For data collected via ¹H NMR, the natural log of the Z isomer concentration was plotted against time to yield a straight line implying first order kinetics. The gradient of this line was used to determine the rate constant for Z→E conversion from which the thermal half-life could be calculated:

$${t}_{1 / 2} = \frac{ln ( 2 )}{k}$$

For data collected via UV-Vis Spectroscopy, absorbance data at a fixed wavelength was fitted using the following, first order equation:

$$ln \frac{{A}_{∞} - {A}_{0}}{{A}_{∞} - {A}_{t}} = kt$$

where k represents the rate constant; A_{∞}, A₀ and Aₜ are the absorbances of the pure E-state, Z-rich state, and time t, respectively. The gradient of this line was used to determine the rate constant for Z→E conversion from which the thermal half-life could be calculated, as per above.

### Zebrafish

Zebrafish (Danio rerio) wild-type TuAB or trpa1b mutant [35] larvae were used for all experiments. Zebrafish embryos were produced using group mating of adult zebrafish. Larvae were raised in E3 media 1×E3 media (0.68 mM NaCl, 0.18 mM KCl, 0.33 mM CaCl₂, 0.4 mM MgCl₂) and maintained in an incubator with a 14 h light/10 h dark cycle at 28.5° C. until experiments. The maintenance of adult animals, obtaining of embryos and larvae, and all experimental procedures were carried out according to protocols approved by the University of Utah's Institutional Animal Care and Use Committee (IACUC).

### Chemical Libraries and Treatments

A total of 1,000 compounds were selected from the ChemBridge Corp. catalog. Compounds were dissolved in DMSO at a stock concentration of 1 μg/μL (˜1.5 mM). The library was screened at a 1:100 dilution in E3 solution for a final concentration of ˜15 μM. Negative controls were treated with an equal volume of DMSO. Groups of 3 larvae at 3 days post fertilization (dpf) were distributed into the wells of 96-well clear bottom black microplates (07-200-567; Fisher Scientific) in 150 μL E3 before the addition of small molecules. Stock solutions of compounds were added directly to zebrafish in the wells of a 96 well plate (Corning 3631), mixed, and allowed to incubate for 1 h in the dark at 28.5° C. prior to behavioral evaluation in the behavioral assay. Ordering information: 1 (5533696; ChemBridge); 3 (5538408; ChemBridge).

### Behavioral Assay

Larvae in each of the wells were exposed to four 1 second flashes of light stimulus in the following order: 450-500 nm (WL1), 415-455 nm (WL2), 352-402 nm (WL3), white light (WL4), with a 5 second inter-stimulus interval. Digital video recording was performed for 5 seconds before the first light stimuli and continued throughout the stimulation sequence. Band pass filters (Semrock FF02-475/50, FF02-435/40, FF01-377/50) were used to restrict the excitation light to the indicated wavelengths. Light-induced motion response was used as the assay readout as DMSO treated control larvae do not respond to light stimulus. 290 frames of digital video were recorded per well at 10 FPS using an EMCCD camera (C9100; Hamamatsu) mounted on an inverted compound microscope (AxioObserver A1; Zeiss) with a NA 0.03 1.25× objective and a barrier with a 0.7 mm diameter opening to restrict light scattering to the sample. MetaMorph software (Molecular Devices) was used to control the execution of TTL signals and camera capture using the built-in stream acquisition with trigger function. Each video was saved for review. Light stimuli were generated with an ozone free 300-Watt xenon bulb housed in a Lambda LS illuminator (Sutter Instruments). A dichroic mirror (T510LPXRXT; Chroma) was used for appropriate excitation of samples and bright field acquisition. Schott longpass absorption glass (RG610; Chroma) was added in the transmitted light path to reduce unwanted excitation to the well and to provide sufficient light for video recording. All behavioral experiments were conducted at room temperature.

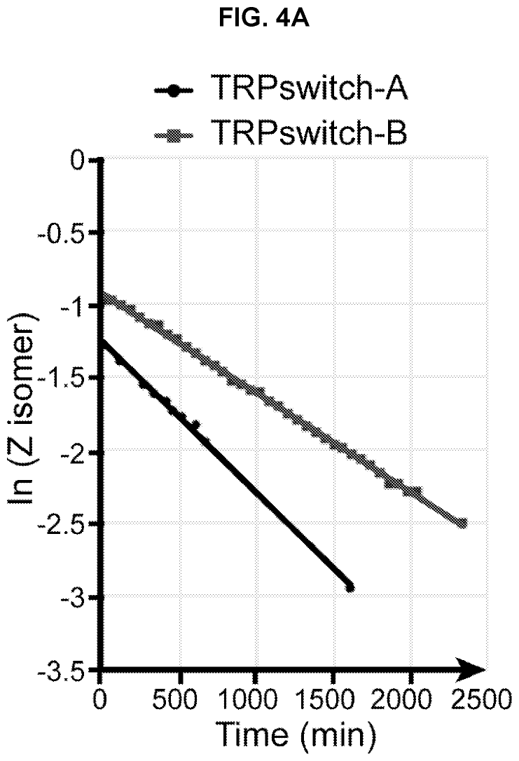

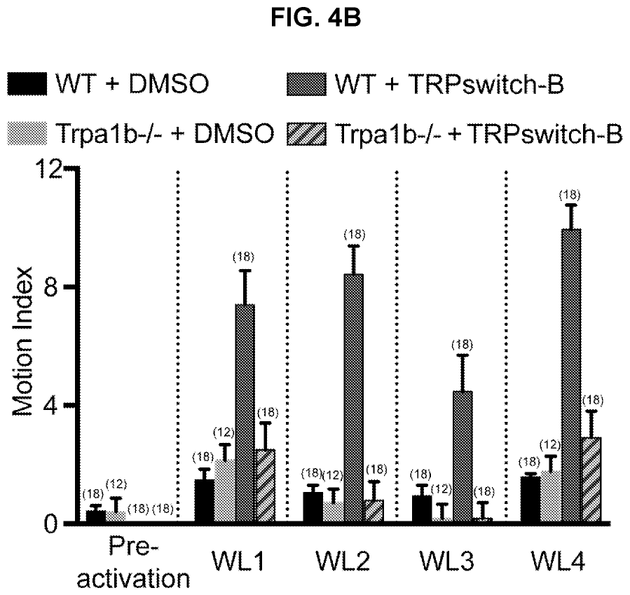

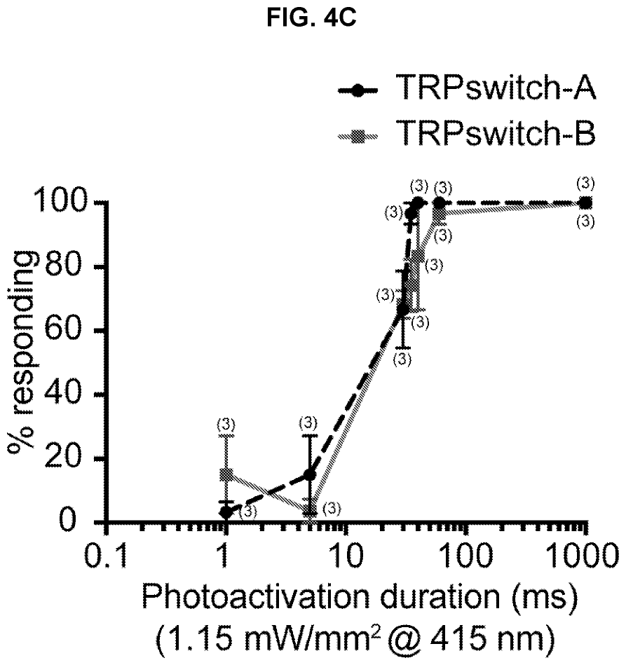

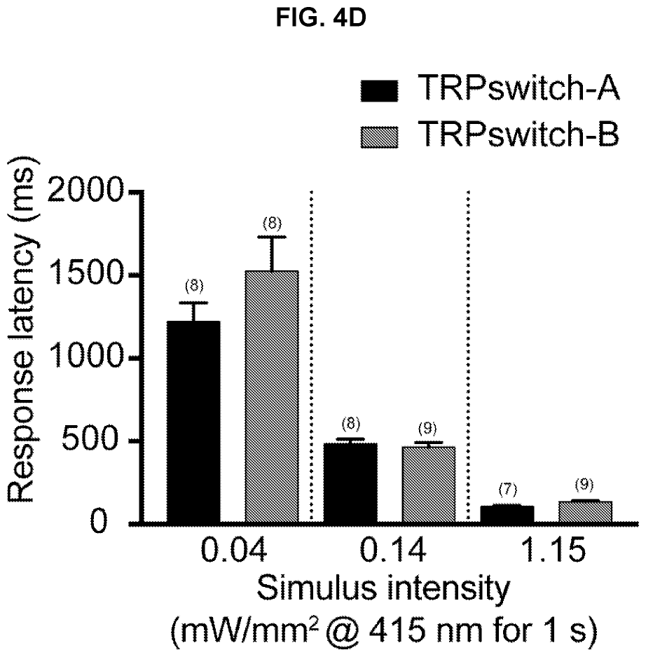

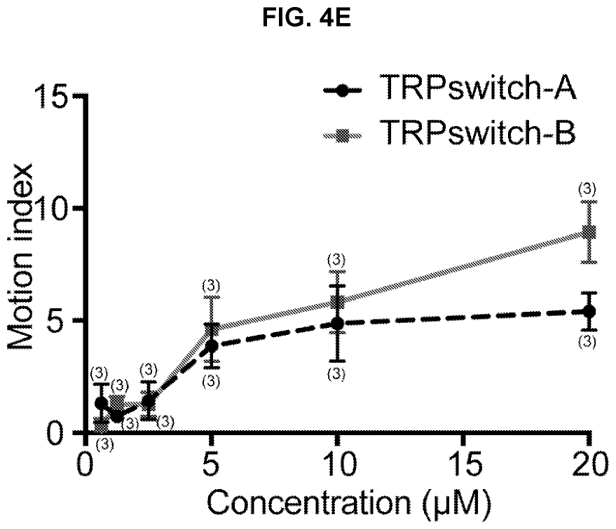

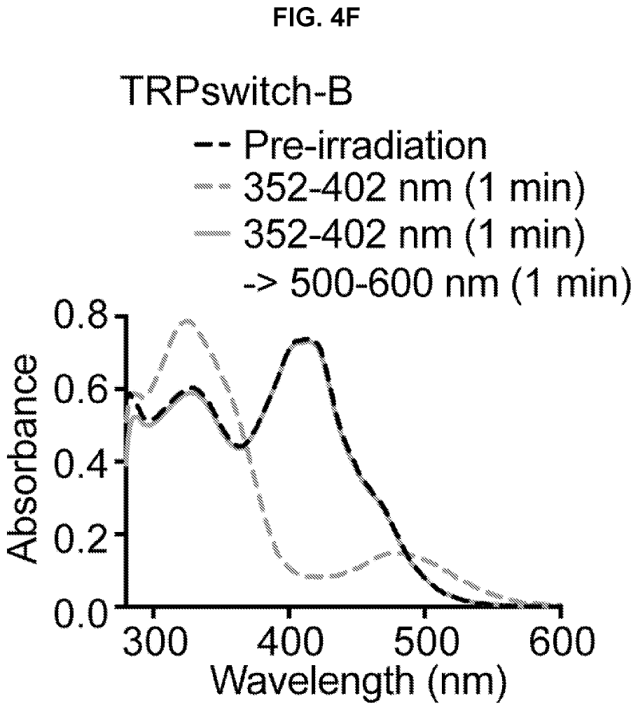

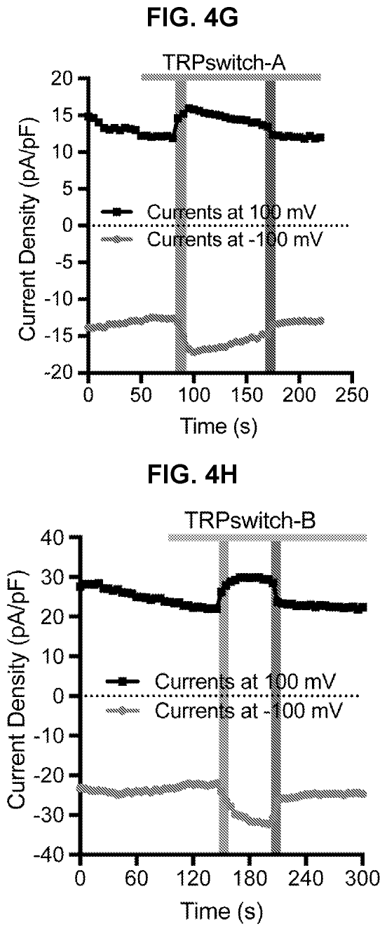

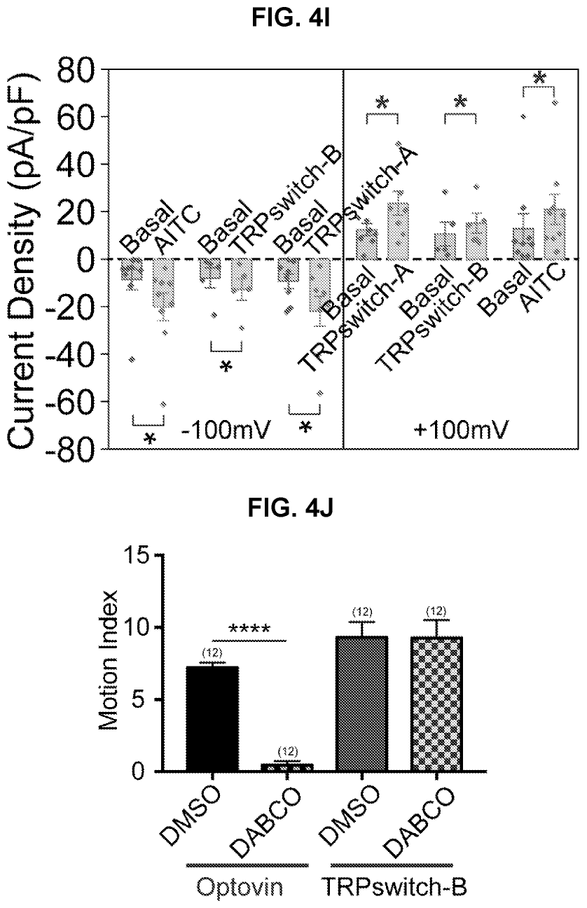

For FIGS. 4C and 4D, experiments were performed using an inverted compound microscope (AxioObserver A1; Zeiss) equipped with an EMCCD camera (C9100; Hamamatsu), a violet LED light source (415 nm) with a CW 310 mW maximum output power source (BLS-LCS-0415-03-22; Mightex) which was controlled by a BioLED light source control module (BLS-SA02-US; Mightex), and a pulse master multi-channel stimulator (A30; World Precision Instruments). Bright field time-lapse was captured for 500 frames at ˜103 FPS. A light pulse was applied at frame 20. For FIG. 4C, percentage responding was quantified based on whether there was any motion in the entire acquisition period. For FIG. 4D, response time is the duration from the beginning of the light pulse to the first movement of the larvae. Light intensity was measured using a hand-held laser meter (LaserCheck, Coherent 1098293).

For Trpa1b ohnolog and paralog rescue experiments, Trpa1b^{-/-} embryos were injected with ngn1:zTrpa1b-2A-mCherry (partial plasmid sequence in SEQ ID NO:5), ngn1:zTrpa1a-2A-mCherry, ngn1:hTRPA1-2A-EGFP or ngn1:mTRPA1-2A-mCherry at the 1-cell stage for mosaic Rohon-beard neuron expression. Embryos were screened at 2 dpf for fluorescent expression in Rohon-beard neurons, incubated with 20 μM TRPswitch-B for 1 hr and decapitated posterior to the eyes right before experiments. NA 0.25 5× objective and 1 s WL3 light illumination was used.

### Behavioral Analysis

To analyze digital video recordings, custom MetaMorph software scripts were used to automatically threshold the video to identify the area of larvae in each frame. Threshold images of the frames after each light activation event were overlaid to calculate a new combined threshold area. The motion index was calculated as the percentage change between the threshold area in the frame right before light activation and the new combined threshold area. This motion index correlates with the overall amount of motion in the well.

### Electrophysiology Experiments

Immortalized HEK293T cells or human colonic fibroblast cells (CCD-18Co; ATCC) were used. HEK293T cells were plated on 12 mm² cover slips and transiently transfected with 10 μg of Trpa1b plasmid (pCMV-zTrpa1b-FLAG) (SEQ ID NO:3) and 5 μg of mNeonGreen cDNA (Allele Biotechnology), then grown in a 6 cm plate for 24-48 h. CCD-18Co were cultured in Eagle's minimal essential medium with 10% FBS, 1× penicillin and streptomycin. CCD-18Co cells were plated on 12 mm cover slips and transiently transfected with 5 μg of zTrpa1b plasmid (pCMV-zTrpa1b-FLAG) together with 5 μg of mNeonGreen cDNA (Allele Biotechnology), then grown in a 10 cm³ plate for 48-72 h. After this time, the cover slips were transferred to a recording chamber containing an extracellular solution composed of 145 mM sodium gluconate, 4 mM KCl, 3 mM MgCl₂, 10 mM D-glucose, 10 mM HEPES; pH 7.4 adjusted with NaOH. The internal solution contained 122 mM cesium methane sulfonate, 1.8 mM MgCl₂, 9 mM EGTA, 14 mM creatine phosphate (sodium salt), 4 mM Mg-ATP, 0.3 mM Na-GTP, 10 mM HEPES, pH 7.2 adjusted with CsOH. Borosilicate glass pipettes with a resistance of 3-5 MO were used. Whole cell currents were measured on an Axopatch 200B amplifier (Molecular De-vices) under the control of pClamp software. Signals were digitized through a Digidata1550B interface (Molecular Devices). Currents were filtered at 5 kHz (lowpass, Bessel) and sampled at 10 kHz prior to analysis with Clampfit software (Molecular Devices). All the plots and statistical tests were performed on Excel (Microsoft corp.). Basal Trpa1b whole cell currents were measured with a ramp protocol (˜100 to +100 mV, at a holding potential of 0 mV). Photoactive compounds were perfused into the recording chamber and a fluorescent light generated by mercury vapor short arc (U-HGLGPS, OLYMPUS) filtered through an ET-ECFP 434/17 nm filter and through a ET-mCherry 560 nm filter (Chroma Technology) was switched “on” and “off” for 10 s. All experiments were performed at room temperature. The p values were calculated using a two-tailed, paired Student's t-test.

### Heart Experiments

Heartbeat interruption experiments were performed in vivo on 2 dpf Tg(cm/c2:Trpa1b-2A-EGFP) [24] larvae in a Trpa1b^{-/-} background. Larvae were pretreated with 20 μM of TRPswitch-A or TRPswitch-B in E3 at a final concentration of 1% DMSO for 1 h in the dark at 28.5° C. before experimental manipulation. Immediately prior to the onset of experiments, E3 solution was replaced with 0.2 mg/mL tricaine (Sigma, A5040) in E3 for anesthetizing the larvae. Violet light (352-402 nm) and green light (500-600 nm) were used for E to Z and Z to E isomerization, respectively. Violet and green light excitation were achieved using a band pass filter FF01-377/50 and an et500lp long pass barrier filter, respectively, together with a t600plxxr dichroic mirror and a 300-Watt xenon bulb light source (Sutter Instrument). Experiments were performed using a NA 0.6 40× air objective at room temperature. The zebrafish heart in the whole field of view was illuminated with 1 s violet light followed by various lengths of green light as indicated. Ventricle width measurement was performed using the ImageJ ‘measure’ function on the widest outer ventricle width every 100 ms.

### Example 3

### UV-Vis Measurements

Compounds were dissolved in DMSO at 250 μM and 260 μM for TRPswitch-A and TRPswitch-B, respectively and UV-Vis absorbance was measured using a NanoDrop 1000 (Thermo Scientific).

### Photostationary State (PSS) Determination

The PSS composition at 420 nm (LZC-420), 365 nm (3×800 mW Nichia NCSU276A LEDs) and 495 nm (3×750 mW Nichia NCSE119B-V1 LEDs), of the relevant compounds, was determined in both DMSO-d₆ (via ¹H NMR) and a 30% water:DMSO mixture using either ¹H NMR or UV-Vis spectroscopy). In the latter case, the pure Z isomer spectrum was estimated using methods described in previous publications [32, 39, 41].

### Statistical Analyses

All results are expressed as means±SEM. Unless otherwise indicated, a two-tailed un-paired student's t test with Mann-Whitney post-test was used to determine p values. The criterion for statistical significance was p<0.05. Statistical analysis was performed using Prism (GraphPad Software).

### Example 4

### Zebrafish Behavior-Based Chemical Screening Identifies TRPswitch-A

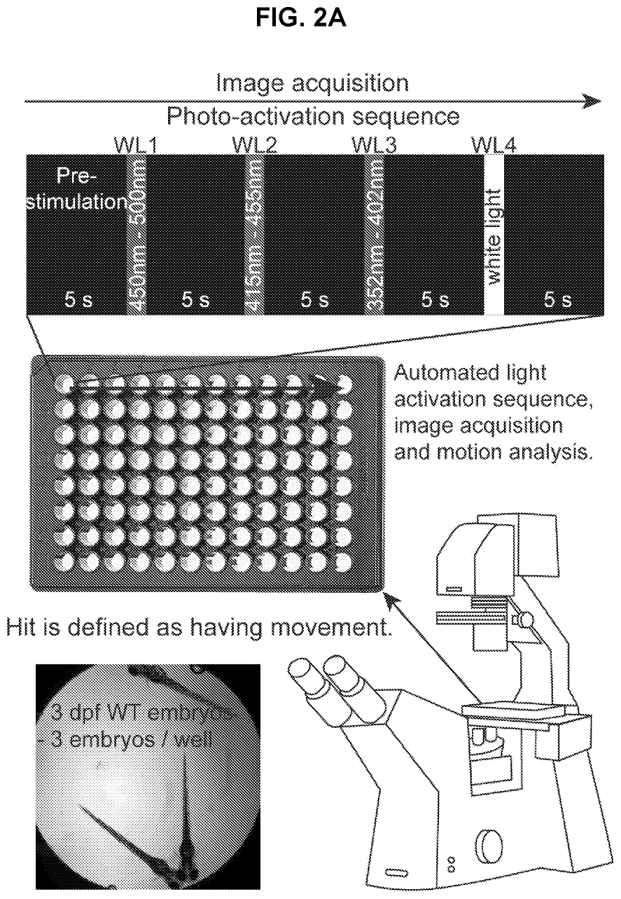

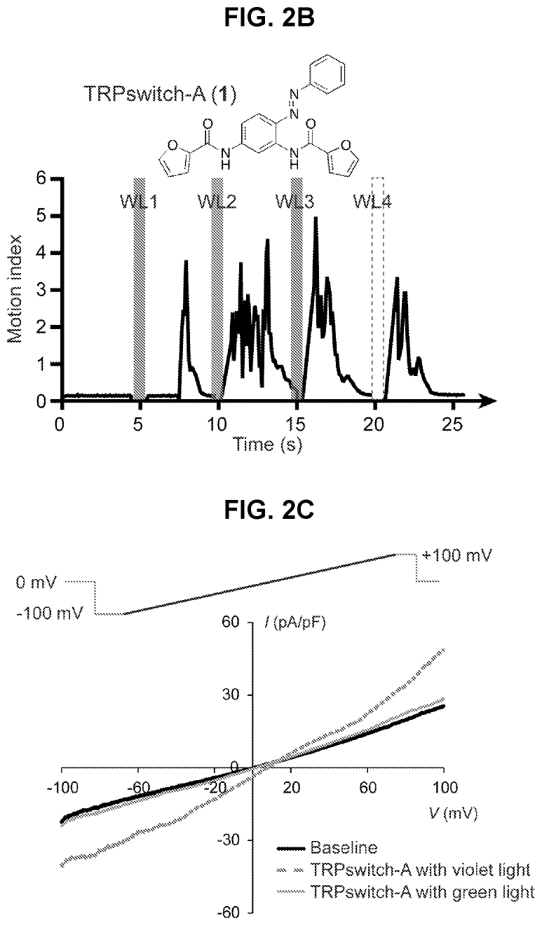

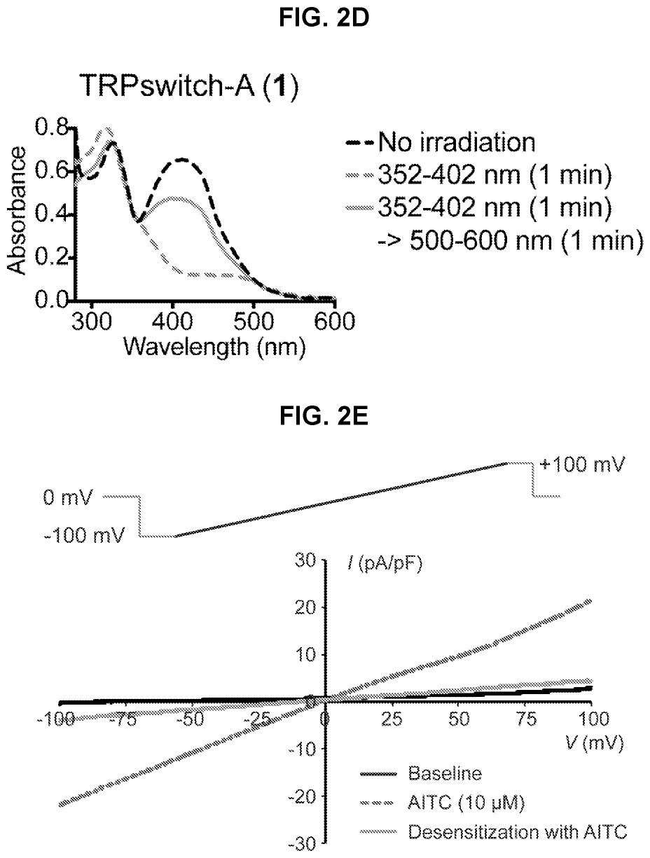

With the discovery of photochromic soluble ligands (PCLs) as a means to control ion channel function using light [11-15] it was reasoned that molecular photoswitches for TRPA1 could be discovered using small molecule screening. To achieve this goal, a modified version of the behavioral assay was developed that was previously utilized to identify photoactivable, but non-photoreversible, ligands of TRPA1 [23]. This medium-throughput, semi-automated screening assay was performed using a 96-well plate format where three larvae per well were incubated with small molecules. A library of 1,000 structurally diverse small molecules enriched for molecular photoswitch moieties such as acylhydrazone [27] azobenzene [28-31], azoheteroaryl [32-33], and stilbene [31, 34] was screened. The particular focus was on molecular photoswitches that operate via a reversible E/Z isomerization using different wavelengths of light. In this assay, each well of a 96-well plate is illuminated with a series of different wavelengths of light (450-500 nm, 415-455 nm, 352-402 nm, and white light) for one second to induce isomerization of the photoswitchable compounds (FIG. 2A). Each illumination event is separated by a dark period of 5 s and the motion of larvae during this light illumination sequence is recorded and analyzed (FIG. 2A).

Wild-type 3-days-post-fertilization larvae (dpf) were used, as they have a relatively developed central nervous system and show no motion response to light exposure at this stage of development. Zebrafish larvae express Trpa1b in a subset of trigeminal and Rohon-Beard sensory neurons [35]. Activation of Trpa1b induces a reproducible and robust motion response [23-24, 35-36]. A light-induced motion response, due to the presence of a photoactivated ligand for ion channels such as Trpa1b, is used as the readout for the assay. DMSO treated larvae on each screening plate served as negative controls. Using this screening assay, TRPswitch-A, an azobenzene containing small molecule was identified with no previously annotated biological activity.

The presence of TRPswitch-A led to a light-induced motion response across multiple wavelengths, with the most activity in a bandwidth between 415-455 nm (WL2 in FIG. 2B). Using whole-cell patch-clamp recordings of HEK293T cells expressing zebrafish Trpa1b, the photocurrents elicited by the Trpa1b/TRPswitch-A pair upon light stimulation were characterized. Violet light stimulation of TRPswitch-A-primed Trpa1b channels generated high amplitude currents when compared to baseline measurements (FIG. 2C). When the same cell was subsequently stimulated with green light, Trpa1b photocurrent recovered to its baseline current magnitude (FIG. 2C). This reversible light response corresponds to the reversible E/Z isomerization of TRPswitch-A observed upon illumination with violet and green light, as judged by UV-Vis absorbance measurements (FIG. 2D). Expression of functional Trpa1b channels in HEK293T cells was confirmed by the observance of allyl isothiocyanate (AITC)-activated currents (FIG. 2E), as AITC is a potent TRPA1 agonist. Desensitization of AITC-induced current over time was observed, further confirming the activity of zTrpa1b channels (FIG. 2E). Overall, these data indicate that TRPswitch-A is a light-dependent and light-reversible activator of Trpa1b.

### Example 5

### TRPswitch-A Structure Activity Relationship Analysis

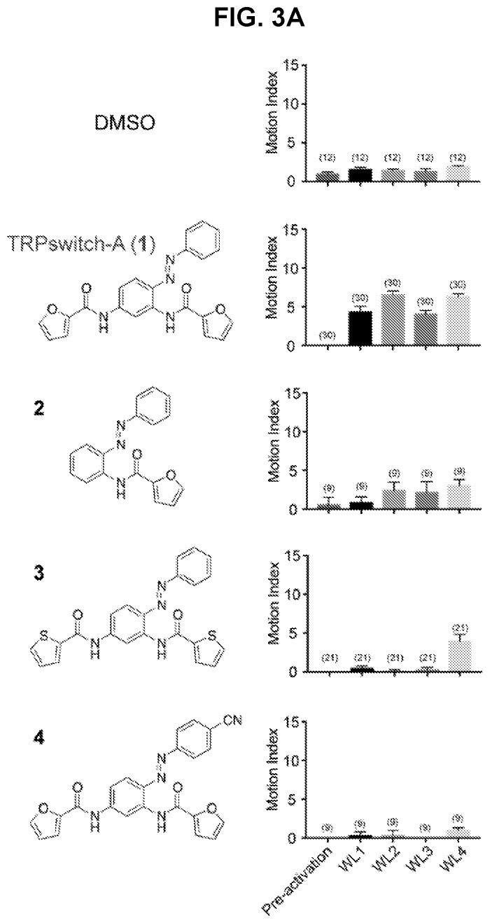

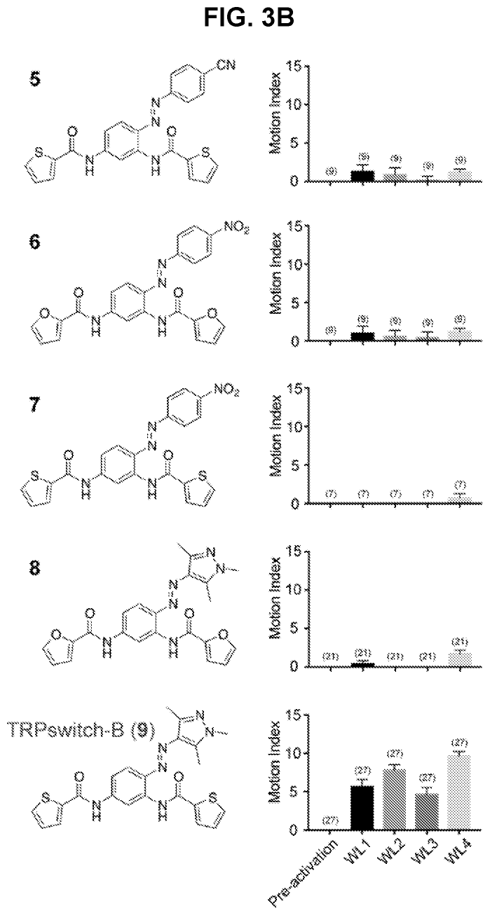

The TRPswitch-A chemical structure contains an azobenzene and a 2-furamide group in both the ortho and para positions of one of the benzene rings (Table 1, Compound 1; FIG. 3A). It is clear that while TRPswitch-A undergoes reversible E/Z isomerization upon illumination with violet and green light, the Z→E photoswitching event is incomplete (FIG. 2D). The chemical features responsible for TRPswitch-A's biological activity and how photoswitch performance correlates to the biological effects observed were investigated. Derivatives of TRPswitch-A were designed, synthesized, and the structure activity relationships was analyzed. The para amide is needed for Trpa1b activation as suggested by the reduced activity of Compound 2 (FIG. 3A). Substituting the furan for thiophene was tolerated (FIG. 3A, Compound 3), albeit with a slightly lower photoresponse which might be due to the overall chemical structural change. Derivatives bearing para electron-withdrawing groups—to increase the “push-pull” character [37-38]-were prepared (Compounds 4, 5, 6, 7) and found to be far less active in the assay (FIG. 3A-FIG. 3B). This poor activity may, in part, be ascribed to the incomplete E-Z photoswitching of these compounds (Table 2). Azoarene performance can be improved/tuned by substituting one of the benzene rings in a conventional azo-benzene for a 5-membered heteroaromatic ring. Specifically, the azopyrazoles show near quantitative photoswitching in both directions and exhibit long Z-isomer thermal half-lives. Replacement of the phenyl ring in Compound 1 (TRPswitch-A) and Compound 3 with a trimethylpyrazole generates Compounds 8 and 9 that undergo quantitative photoswitching in both directions (FIG. 4F) and that have long thermal half-lives. The thermal Z-isomer half-life of Compound 9 (TRPswitch-B), in DMSO-d₆, is 17 hours (FIG. 4A). While some aqueous solubility issues for Compound 8 were observed, which likely limits its biological response, Compound 9 (TRPswitch-B) has a comparable biological response to TRPswitch-A (Compound 1).

TABLE 1
Compounds 1-9

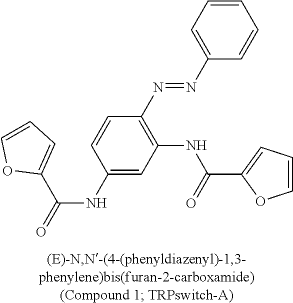 [SMILES: O=C(Nc1ccc(/N=N/c2ccccc2)c(NC(=O)c2ccco2)c1)c1ccco1]

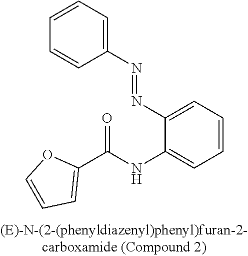 [SMILES: O=C(Nc1ccccc1/N=N/c1ccccc1)c1ccco1]

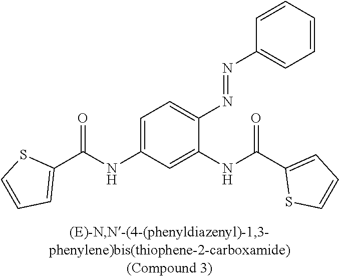 [SMILES: O=C(Nc1ccc(/N=N/c2ccccc2)c(NC(=O)c2cccs2)c1)c1cccs1]

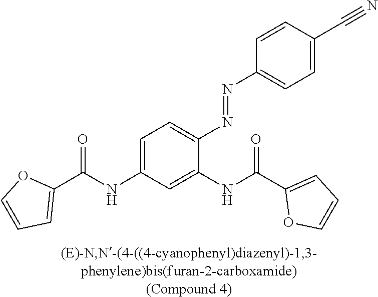 [SMILES: N#Cc1ccc(/N=N/c2ccc(NC(=O)c3ccco3)cc2NC(=O)c2ccco2)cc1]

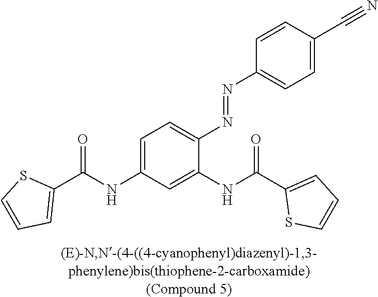 [SMILES: N#Cc1ccc(/N=N/c2ccc(NC(=O)c3cccs3)cc2NC(=O)c2cccs2)cc1]

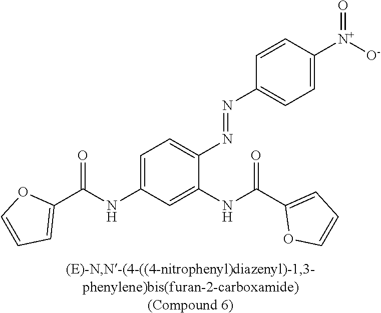 [SMILES: O=C(Nc1ccc(/N=N/c2ccc([N+](=O)[O-])cc2)c(NC(=O)c2ccco2)c1)c1ccco1]

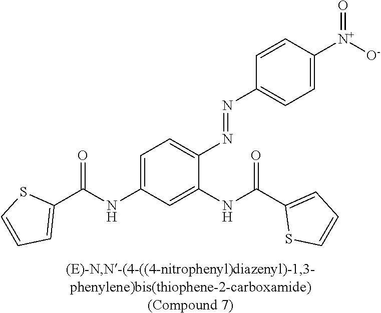 [SMILES: O=C(Nc1ccc(/N=N/c2ccc([N+](=O)[O-])cc2)c(NC(=O)c2cccs2)c1)c1cccs1]

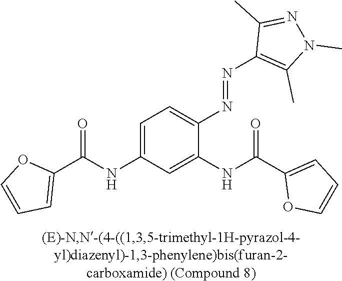 [SMILES: Cc1nn(C)c(C)c1/N=N/c1ccc(NC(=O)c2ccco2)cc1NC(=O)c1ccco1]

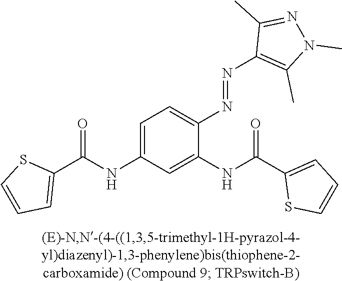 [SMILES: Cc1nn(C)c(C)c1/N=N/c1ccc(NC(=O)c2cccs2)cc1NC(=O)c1cccs1]

TABLE 2
Estimated photostationary states (PSS)
determined by UV/Vis spectroscopy in DMSO

| Compound | 365 nm PSS | 420 nm PSS | 495 nm PSS |
|---|---|---|---|
| 2 | 13% E | 56% E | 79% E |
| 3 | 22% E | 38% E | 76% E |
| 4 | 47% E | 32% E | 61% E |
| 5 | 41% E | 30% E | 61% E |
| 6 | 66% E | 42% E | 64% E |
| 7 | 69% E | 46% E | 70% E |

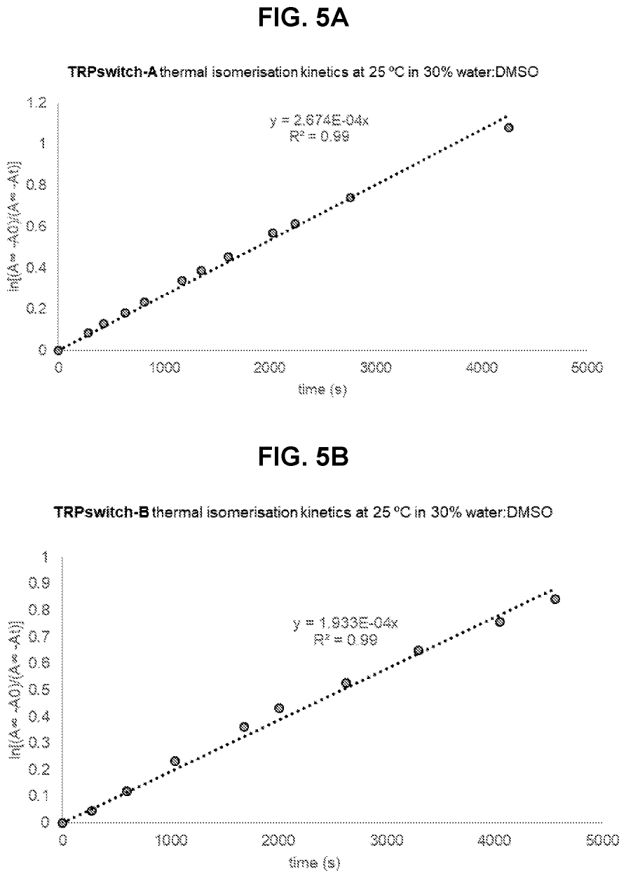

To further characterize the thermal half-lives of TRPswitches in conditions that more closely resemble those used in biological experiments, the thermal half-lives at higher water contents (30% water:DMSO solutions) were measured. The thermal half-lives of TRPswitch-A and -B were 43 min and 1 hour, respectively. When measurements were made in DMSO-d₆, the half-lives for TRPswitch-A and -B were 11 and 17 hours, respectively. The thermal isomerization kinetics were also measured at 25° C. in 30% water:DMSO solutions (FIG. 5A-FIG. 5B).

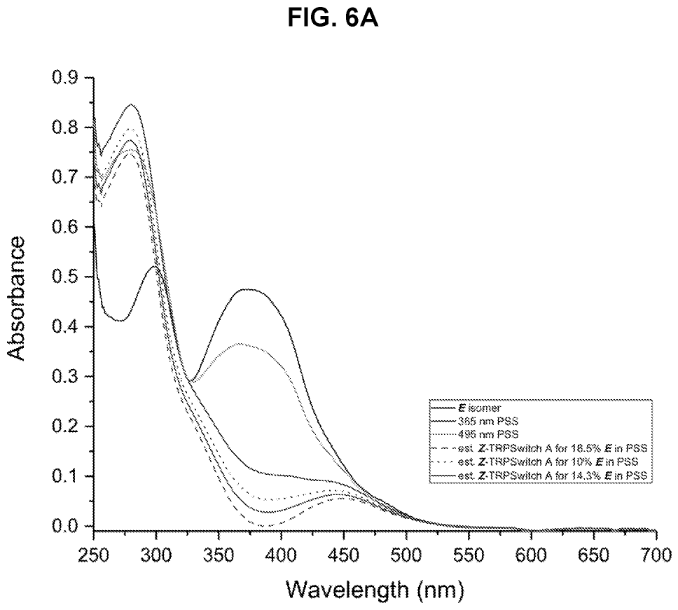

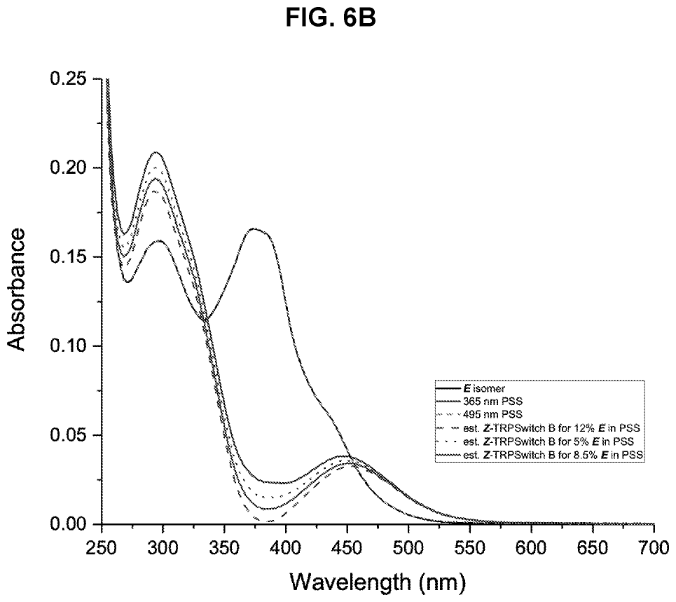

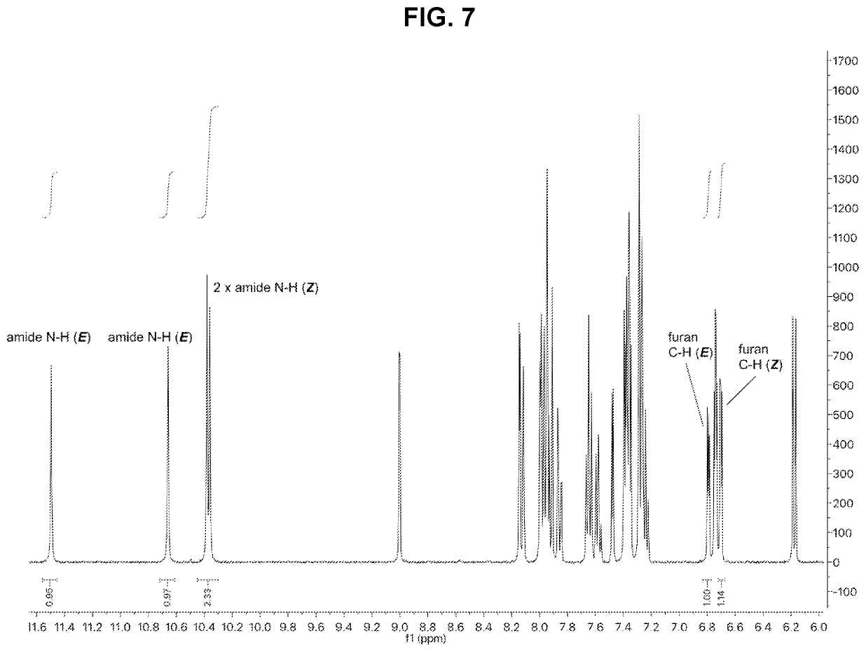

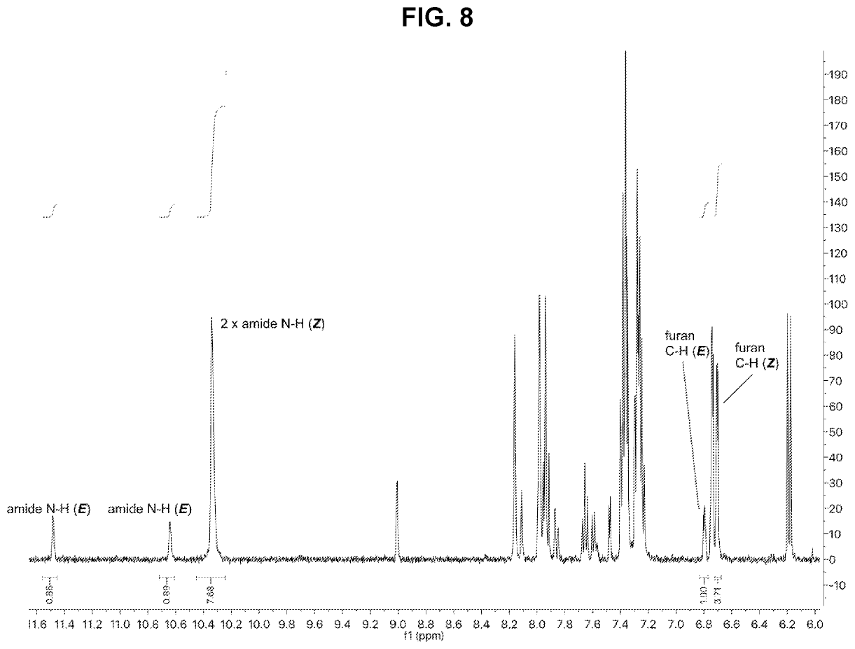

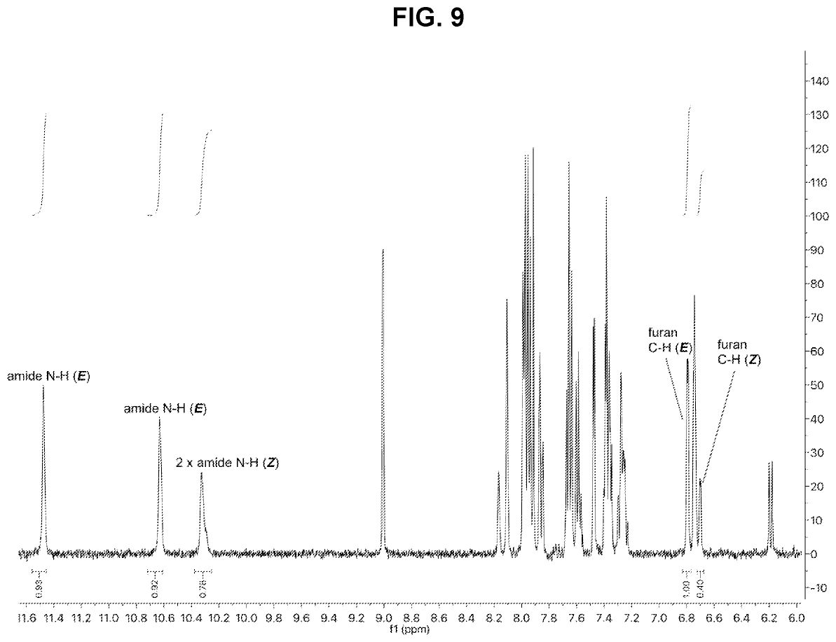

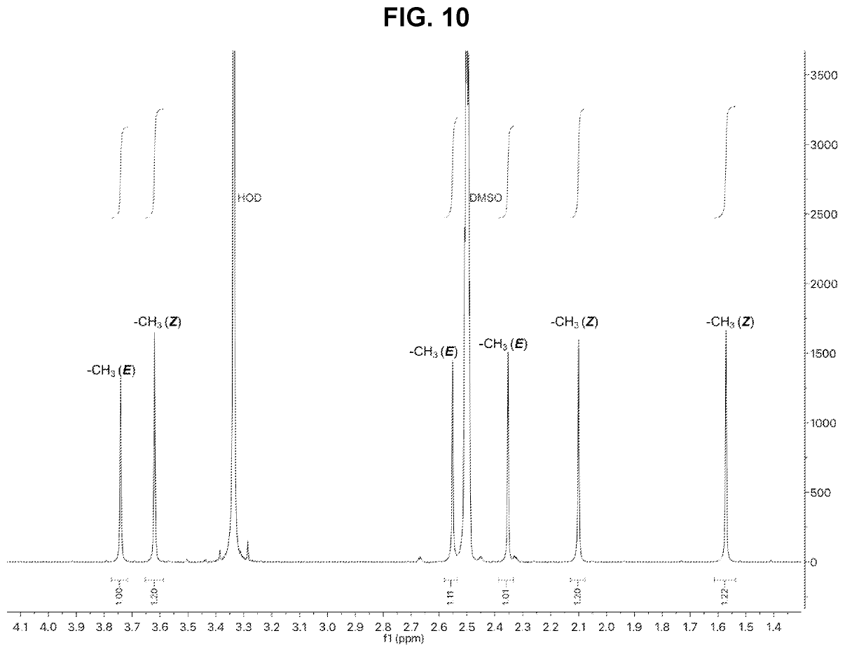

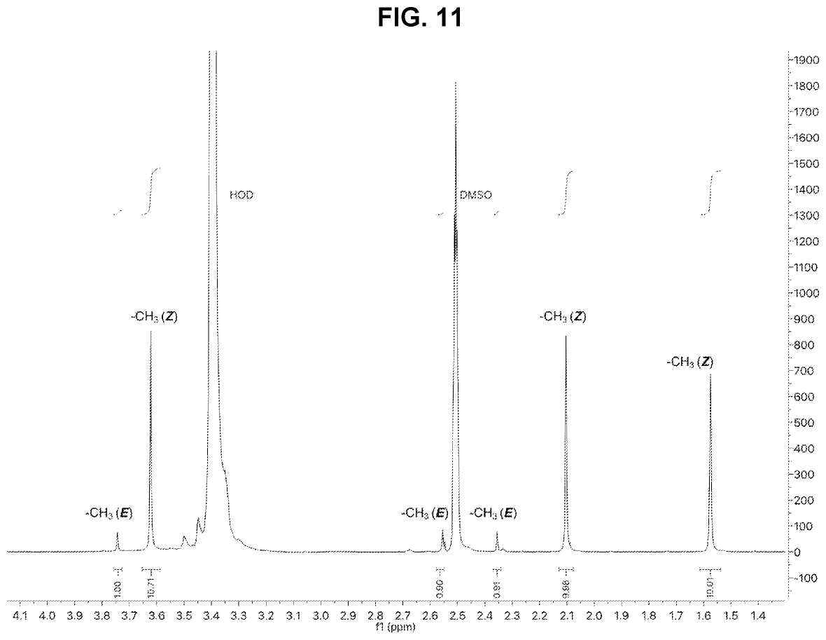

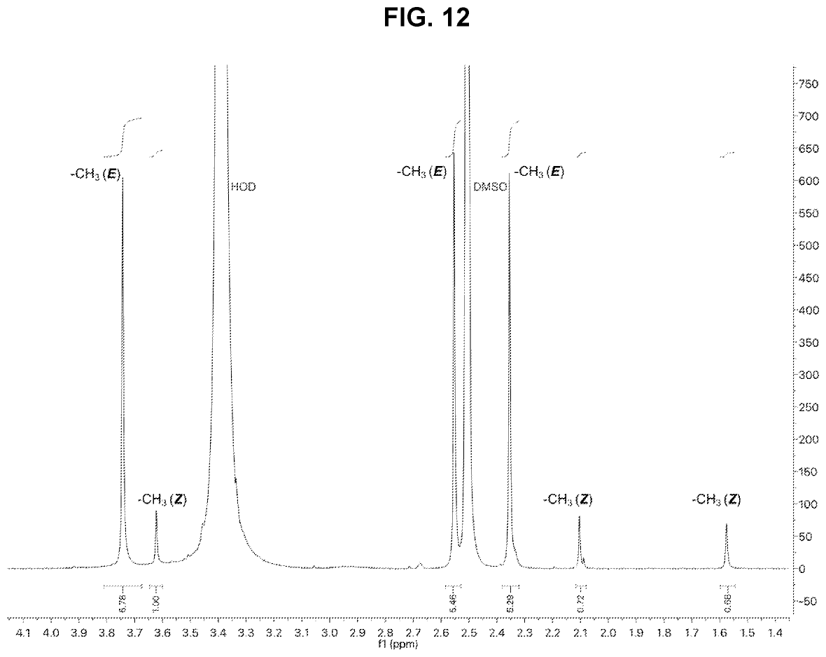

To further characterize the photochemical properties of TRPswitches, the photostationary state (PSS) of TRPswitches in 30% water:DMSO solutions were examined via UV-Vis. By extrapolating a pure Z spectrum, using methodology outlined in previous publications [32, 39, 41], the PSS composition is approximated to be 86% Z at 365 nm and 73% E at 495 nm for TRPswitch-A; and 92% Z at 365 nm and 100% E at 495 nm for TRPswitch-B (FIG. 6A-FIG. 6B, Table 3). In addition, the PSS of TRPswitches was examined in DMSO-d6 at 365, 420, and 495 nm (FIG. 7-FIG. 12, Table 4). From this analysis, TRPswitch-B demonstrates superior, near quantitative photoswitching compared to the azobenzene analogue. These data suggest that photoswitch performance, particularly Z-isomer half-life, contributes to the activity and that both azobenzene (TRPswitch-A) and azoheteroaryl (TRPswitch-B) moieties can be used as photochromic soluble ligands of Trpa1b, with TRPswitch-B an improved intrinsic photoswitch performance. Additionally, the X-ray crystal structure of TRPswitch-B has been obtained (FIG. 1). Notably, this compound crystallizes in a chiral space group, with the azo bridge orientated to take advantage of a favorable hydrogen bonding interaction from the ortho amide.

TABLE 3
Photostationary state (PSS) of TRPswitches determined
by UV-Vis spectroscopy in 30% water:DMSO

| | Estimated 365 nm | Estimated 495 nm |
|---|---|---|
| Compound | PSS (E-Z) | PSS (Z-E) |
| TRPswitch-A | 86% Z | 73% E |
| TRPswitch-B | 92% Z | 100% E |

TABLE 4
Photostationary states (PSS) of TRPswitches
determined by ¹H NMR in DMSO-d₆

| | 365 nm | 420 nm | 495 nm |
|---|---|---|---|
| Compound | PSS (E-Z) | PSS (E-Z) | PSS (Z-E) |
| TRPswitch-A | 79% Z | 53% Z | 71% E |
| TRPswitch-B | 92% Z | 55% Z | 86% E |

### Example 6

### TRPswitch is a Reversible Photoswitch Ligand for Trpa1b

Optovin was previously identified as a photoactivable ligand for the Trpa1b channel and confirmed that its activity is abolished in Trpa1b mutant zebrafish. To determine if Trpa1b is necessary for TRPswitch's biological activity, a behavioral assay using Trpa1b mutant larvae was performed [35]. When Trpa1b mutant larvae were used, the TRPswitch light-induced motion response was abolished (FIG. 4B). This suggests that Trpa1b is required for the activity of TRPswitch in vivo, consistent with the electrophysiology analysis (FIG. 2C). Both TRPswitch-A- and TRPswitch-B-treated larvae showed a higher probability of light-induced motion response as the light stimulation duration increased (FIG. 4C). The probability for larvae to respond to light reached its maximum with a <1 s light-pulse length. The biological response triggered by TRPswitch/Trpa1b activation was very rapid. The latency to motion response from the introduction of light was in the range of milliseconds and decreased with increasing light intensity (FIG. 4D). A positive correlation was observed between the activity of TRPswitch and an increase in compound concentration, with maximum activity achieved at concentrations between 10-20 μM (FIG. 4E). Similar to TRPswitch-A, TRPswitch-B undergoes reversible E/Z isomerization upon illumination with violet and green light. However, unlike TRPswitch-A, the Z-to-E conversion by green light leads to complete return to the pre-illumination “off” state (FIG. 4F).

To characterize the stability and kinetics of Trpa1b/TRPswitch-dependent photocurrents, zebrafish Trpa1b activity was recorded in HEK293T cells. The light-induced activation and deactivation of Trpa1b/TRPswitch was triggered with violet and green light pulses, respectively (FIG. 4G and FIG. 4H). Importantly, the photocurrents were sustained after the initial short pulse of violet light illumination and did not require continuous illumination. The photocurrents could be converted back to baseline using a subsequent pulse of green light. The current density fold increments of TRPswitch-A and TRPswitch-B upon violet light illumination were 2.36±0.50 and 2.12±0.47 at +100 mV and 4.35±1.44 and 2.27±0.38 at −100 mV, respectively. A subsequent pulse of green light reduced the current density fold increments of TRPswitch-A and TRPswitch-B to 1.15±0.17 and 1.26±0.33 at +100 mV and 3.71±1.31 and 1.41±0.38 at −100 mV, respectively. Together, these data indicate that TRPswitch-A and TRPswitch-B are novel, reversible photoswitches that act on the Trpa1b channel. Overall, the light-activated currents with TRPswitches are comparable to those induced by the canonical TRPA1 agonist AITC (FIG. 4I).

To test the potential cross activity of TRPswitch-B on the zebrafish ohnolog Trpa1a and on mammalian orthologs, zebrafish Trpa1a, mouse TRPA1 or human TRPA1 were transiently re-expressed in the Rohon-beard neurons of mutant Trpa1b^{-/-} zebrafish and performed light-induced motion response experiments in the presence of TRPswitch-B. Rescue experiments were performed in Trpa1b^{-/-} zebrafish with transient mosaic re-expression of zebrafish Trpa1b (zTrpa1b), zebrafish Trpa1a (zTrpa1a), mouse TRPA1 (mTRPA1) or human TRPA1 (hTRAP1) in Rohon-beard neurons. Embryos were pre-treated with 20 μM TRPswitch-B and decapitated right before light stimulation to eliminate the light response mediated by the retina (Table 5). Percent response is the percentage of embryos that exhibited a light-induced motion response within 5 s after the 1 s WL3 illumination. Only the re-expression of zTrpa1b (SEQ ID NO:6) in Rohon-beard neurons in Trpa1b^{-/-} embryos rescued the TRPswitch-B-mediated light-induced motion response. N is the number of embryos tested. Only re-expression of Trpa1b rescued the light-induced motion response of Trpa1b^{-/-} mutants (Table 5). These results suggest that TRPswitch-B is specific to the zebrafish Trpa1b channel.

TABLE 5
Activity of TRPswitch-B (Compound 9)
on Trpa1b ohnolog and orthologs

| | Trpa1b^{-/-} with transient expression of | | | | | |
|---|---|---|---|---|---|---|
| | zTrpa1b | zTrpa1a | mTRPA1 | hTRPA1 | Trpa1b^{-/-} | WT |
| % response | 38.5 | 0 | 0 | 0 | 0 | 65 |
| N | 26 | 16 | 14 | 42 | 16 | 20 |

### TRPswitch's Mechanism of Action

Most known activators of the TRPA1 channel are electrophilic ligands that covalently modify cysteines in TRPA1's cytoplasmic domain. Optovin, the previously identified photoactivable TRPA1 ligand, reacts with those cysteines through a photochemical reaction involving the generation of singlet oxygen species. DABCO, a singlet oxygen quencher and triplet energy acceptor can completely suppress the optovin response [23]. To determine whether or not TRPswitch is also photoactivated in a singlet oxygen-based mechanism, the ability of DABCO to suppress TRPswitch activity was tested and determined that it did not (FIG. 4J). These data suggest that light-induced generation of singlet oxygen is not necessary for TRPswitch's behavioral effect. As the TRPswitches contain either azobenzene or azopyrazole groups, it is likely that their activity is due to the E/Z isomerization of the compounds upon light illumination (FIG. 2D and FIG. 4F), without the production of radicals. These observations suggest that both TRPswitch-A and —B are reversible photoswitches for the Trpa1b channel and have a distinct mechanism of activation as compared to electrophilic ligands.

### Example 7

### Trpa1b/TRPswitch Allows Cellular Activation in Non-Neuronal Cells

To demonstrate the practical utility of TRPswitch in vivo, a heartbeat interruption experiment was performed using a zebrafish transgenic line expressing Trpa1b in cardiomyocytes, Tg(cm/c2:Trpa1b-2A-EGFP), and by applying pulses of violet and green light to the zebrafish heart. Since proper calcium handling is important for normal heartbeat, heartbeat was used as the biological readout for the activation and deactivation of Trpa1b channels. During regular heartbeat cycles, an increase in intracellular calcium is required for cardiomyocyte contraction as calcium binding to troponin C leads to a conformational change that displaces tropomyosin from the actin binding sites. Calcium levels must then decrease for cardiomyocyte relaxation as calcium prevents tropomyosin from returning to its original conformation. It is hypothesized that a sustained elevation of intracellular calcium, such as with TRPA1 activation, will result in a sustained tetanic contraction of the heart and interrupt rhythmic beating.

In transgenic larvae treated with either TRPswitch-A or TRPswitch-B, the ventricle heartbeat could be stopped and would persist in a sustained systolic state after a brief 1 s illumination with violet light (FIG. 13A-FIG. 13D). Normal ventricular rhythm was re-stored after briefly illuminating the heart with 1 s of green light (FIG. 13A-FIG. 13D). These data suggest that the reversible E/Z isomerization of the TRPswitches by violet and green light induced in vivo activation and deactivation of Trpa1b channels, respectively. There was no significant difference in heart rate among transgenic larvae treated with DMSO control or either TRPswitch in the dark, suggesting that the TRPswitches have no effect on Trpa1b channels without light illumination. Treating larvae that lack exogenous expression of Trpa1b in cardiomyocytes with TRPswitch-B and light illumination resulted in similar negative results, indicating that the switching off and on of heartbeat with violet and green light illumination was due to TRPswitch isomerization and the subsequent effect on Trpa1b channel activity.

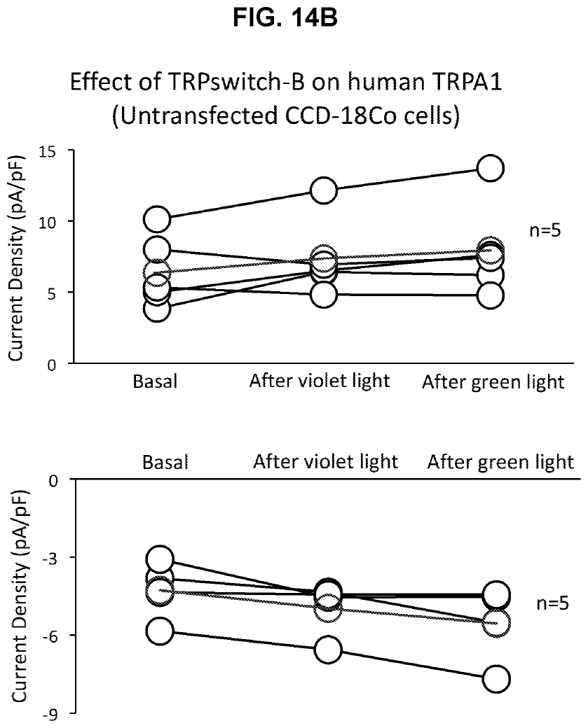

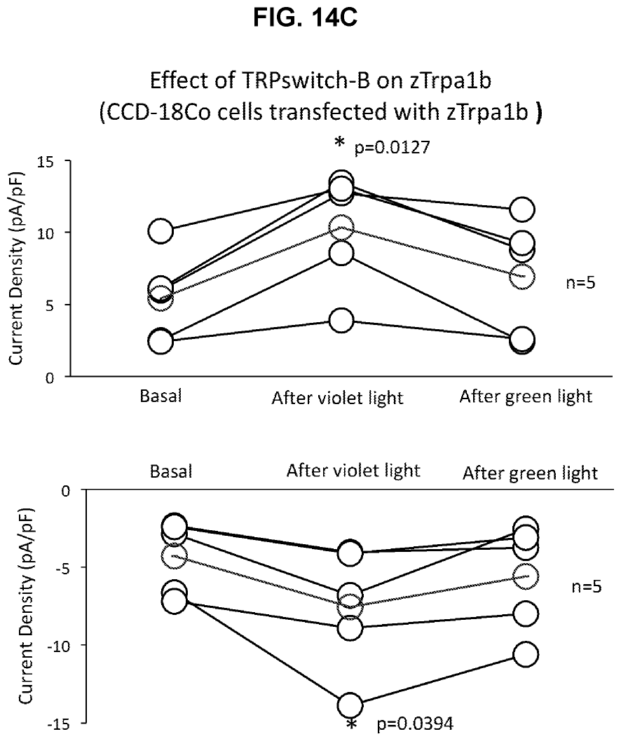

Photoswitchable control of Trpa1b activity by the TRPswitches was repeatable. Cyclical rounds of illumination using violet and green light induced the stopping and restarting of the ventricle heartbeat (FIG. 13C and FIG. 13D). Additionally, channel activation after a brief pulse of violet light was sustained on a timescale of minutes, as measured in the gradual relaxation of ventricle width over time (FIG. 13E). As a further demonstration of the utility of the heterologously expressed zTrpa1b/TRPswitch system, zTrpa1b was expressed in human colonic fibroblast cells, CCD-18Co, which express human TRPA1 endogenously [43]. The expression of human TRPA1 channel in CCD-18Co cells was verified with RT-PCR. CCD-18Co cells responded to AITC, confirming the functional expression of the human TRPA1 channel (FIG. 14A). As expected, TRPswitch showed no light induced photocurrent in CCD-18Co cells without the expression of zTrpa1b (FIG. 14B). When CCD-18Co cells were transfected with zTrpa1b and incubated with TRPswitch-B, they exhibited violet light-induced photocurrent and green light-induced decrease in photocurrent (FIG. 14C). These data provide further evidence that TRPswitch-B has specific activity on zTrpa1b and does not cross react with endogenously expressed human TRPA1 channel. Taken together, these data suggest that the TRPA1/TRPswitch pair constitutes a reversible and repeatable chemo-optogenetic system that is compatible with use in zebrafish and mammalian cells.

1. A photoactive compound of Formula (I): 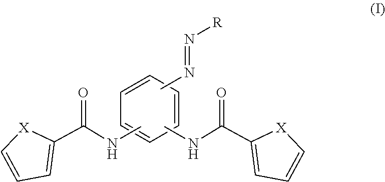
wherein each X is independently O or S and R is a substituted or unsubstituted heteroaryl moiety or a substituted phenyl moiety.

2. The compound of claim 1, wherein the compound is one of Formulae (II), (III), or (IV): 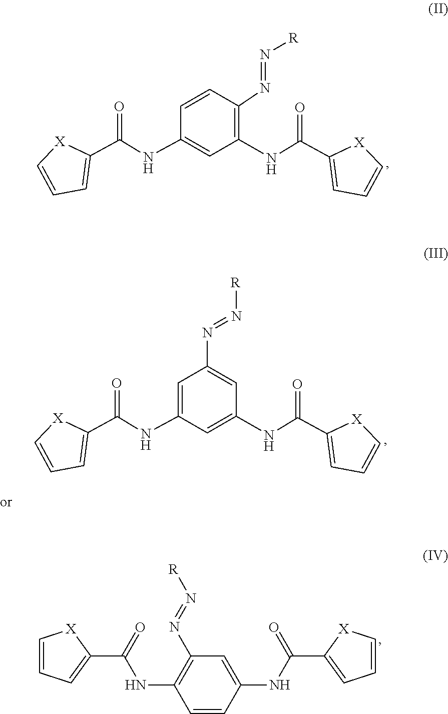
wherein each X is independently O or S and R is a substituted or unsubstituted heteroaryl moiety or a substituted phenyl moiety.

3. The compound of claim 1, wherein each X is independently O or S and
R is a mono- or bi-cyclic aryl ring or a 5-10 membered mono or bi-cyclic heteroaryl ring optionally substituted with one or more of Q₁-(R₁)ₙ;
Q₁ is a covalent bond, H, O, halogen, cyano, —NR₃—, —CONR₂—, —NR₂CO—, oxo, nitro, —S(O)ₘ, —C₁₋₆ haloalkyl, —C₁₋₆ alkoxy, —C₁₋₆ haloalkoxy, —C₁₋₆ hydroxyalkyl, —C₁₋₆ cyanoalkyl, —CO—, —SO₂R₃, —NR₃R₄, —NR₃COR₄, —NR₂CONR₃R₄, —CONR₃R₄, —CO₂R₃, —NR₃CO₂R₄, —SO₂NR₃R₄, —CONR₃, —C(O)R₃, —NR₃SO₂R₄, —NR₂SO₂NR₃R₄, —SO₂NR₃, optionally substituted —C₁₋₆ alkylene, optionally substituted —C₂₋₆ alkenylene, or optionally substituted —C₁₋₆ alkyl;
R₁ is halogen, oxo, cyano, nitro, optionally substituted —C₁₋₆ haloalkyl, optionally substituted —C₁₋₆ alkoxy, optionally substituted —C₁₋₆ haloalkoxy, optionally substituted —C₁₋₆ alkyl, optionally substituted —C₂₋₆ alkenyl, optionally substituted —C₂₋₆ alkynyl, C₁-C₆ hydroxyalkyl, optionally substituted heterocyclyl, optionally substituted cycloalkyl, optionally substituted heteroaryl, optionally substituted aryl, -Q₂-NR₅CONR₆R₇, -Q₂-NR₅R₆, -Q₂-NR₅COR₆, -Q₂-COR₅, -Q₂-SO₂R₅, -Q₂-CONR₅, -Q₂-CONR₅R₆, -Q₂-CO₂R₅, -Q₂-SO₂NR₅R₆, -Q₂-NR₅SO₂R₆, or -Q₂-NR₅SO₂NR₆R₇;
Q₂ is a covalent bond, —C₁₋₆ alkyl, —C₁₋₆ alkylene, or —C₂₋₆ alkenylene;
R₂, R₃, and R₄ are each independently hydrogen, optionally substituted —C₁₋₆ alkyl, or optionally substituted —C₁₋₆ alkylene;
R₅, R₆, and R₇ are each independently H, optionally substituted —C₁₋₆ alkyl, optionally substituted heterocyclyl, optionally substituted heteroaryl, optionally substituted aryl, or optionally substituted cycloalkyl;
n is 0, 1, 2, 3, or 4; when n is 1, 2, 3, or 4, R₁ is an optionally substituted 3-10 membered heterocyclyl, heteroaryl, aryl, or a mono- or bi-cycloalkyl ring; and wherein n is 0, Q is present and R₁ is absent;
m is 0, 1, or 2; and
any of the compounds designated as “optionally substituted” may be substituted with halogen, —C₁₋₆ alkyl, —C₁₋₆ alkenyl, —C₁₋₆ alkynyl, —C₁₋₆ alkoxy, —C₀₋₆ amine, —C₀₋₆ amide, —C₀₋₆—OH, —C₀₋₆—COOH, —C₀₋₆ CN, or C₁₋₆ halogen.

4. The compound of claim 1, wherein each X is S.

5. The compound of claim 1, wherein each X is O.

6. The compound of claim 1, wherein the compound is Formula (V): 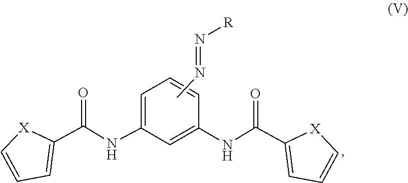
wherein
each X is S or O;
R is 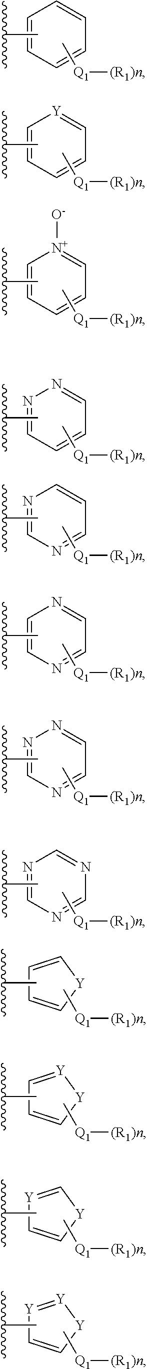 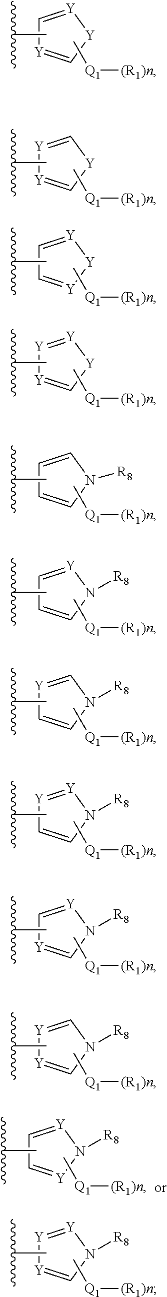
Y is independently O, S, or N;
Q₁ is a covalent bond, H, O, halogen, cyano, —NR₃—, —CONR₂—, —NR₂CO—, oxo, nitro, —S(O)ₘ—, C₁₋₆ haloalkyl, C₁₋₆ alkoxy, C₁₋₆ haloalkoxy, C₁₋₆ hydroxyalkyl, C₁₋₆ cyanoalkyl, —CO—, —SO₂R₃, —NR₃R₄, —NR₃COR₄, —NR₂CONR₃R₄, —CONR₃R₄, —CO₂R₃, —NR₃CO₂R₄, —SO₂NR₃R₄, —CONR₃, —C(O)R₃, —NR₃SO₂R₄, —NR₂SO₂NR₃R₄, —SO₂NR₃, optionally substituted —C₁₋₆ alkylene, optionally substituted —C₂₋₆ alkenylene, or optionally substituted —C₁₋₆ alkyl;
R₁ is halogen, oxo, cyano, nitro, optionally substituted —C₁₋₆ haloalkyl, optionally substituted —C₁₋₆ alkoxy, optionally substituted —C₁₋₆ haloalkoxy, optionally substituted —C₁₋₆ alkyl, optionally substituted —C₂₋₆ alkenyl, optionally substituted —C₂₋₆ alkynyl, —C₁-C₆ hydroxyalkyl, optionally substituted heterocyclyl, optionally substituted cycloalkyl, optionally substituted heteroaryl, optionally substituted aryl, -Q₂-NR₅CONR₆R₇, -Q₂-NR₅R₆, -Q₂-NR₅COR₆, -Q₂-COR₅, -Q₂-SO₂R₅, -Q₂-CONR₅, -Q₂-CONR₅R₆, -Q₂-CO₂R₅, -Q₂-SO₂NR₅R₆, -Q₂-NR₅SO₂R₆, or -Q₂-NR₅SO₂NR₆R₇;
Q₂ is a covalent bond, —C₁₋₆ alkyl, —C₁₋₆ alkylene, or —C₂₋₆ alkenylene;
R₂, R₃, and R₄ are each independently hydrogen, optionally substituted —C₁₋₆ alkyl, or optionally substituted —C₁₋₆ alkylene;
R₅, R₆, and R₇ are each independently H, optionally substituted —C₁₋₆ alkyl, optionally substituted heterocyclyl, optionally substituted heteroaryl, optionally substituted aryl, or optionally substituted cycloalkyl;
R₈ is a covalent bond, hydrogen, halogen, oxygen, oxo, nitro, cyano, —NR₃—, —CONR₃—, —NR₃CO—, —S(O)ₘ—, C₁-C₆ haloalkyl, —C₁-C₆ alkoxy, —C₁-C₆ haloalkoxy, —C₁-C₆ hydroxyalkyl, —C₁-C₆ cyanoalkyl, —CO—, —SO₂R₄, —NR₃R₄, —NR₃COR₄, —NR₂CONR₃R₄, —CONR₃R₄, —C₀₂R₃, —NR₃CO₂R₄, —SO₂NR₃R₄, —CONR₃, —C(O)R₃, —NR₃SO₂R₄, —NR₂SO₂NR₃R₄, —SO₂NR₃, optionally substituted C₁-C₆ alkylene, optionally substituted —C₂-C₆ alkenylene, or optionally substituted —C₁-C₆ alkyl;
n is 0, 1, 2, 3, or 4; when n is 1, 2, 3, or 4, R₁ is an optionally substituted 3-10 membered heterocyclyl, heteroaryl, aryl, or a mono- or bi-cycloalkyl ring; and wherein n is 0, Q is present and R₁ is absent;
m is 0, 1, or 2; and
any of the compounds designated as “optionally substituted” may be substituted with halogen, —C₁₋₆ alkyl, —C₁₋₆ alkenyl, —C₁₋₆ alkynyl, —C₁₋₆ alkoxy, —C₀₋₆ amine, —C₀₋₆ amide, —C₀₋₆—OH, —C₀₋₆—COOH, —C₀₋₆ CN, or C₁₋₆ halogen.

7. The compound of claim 1, wherein each X is S or O and R is: 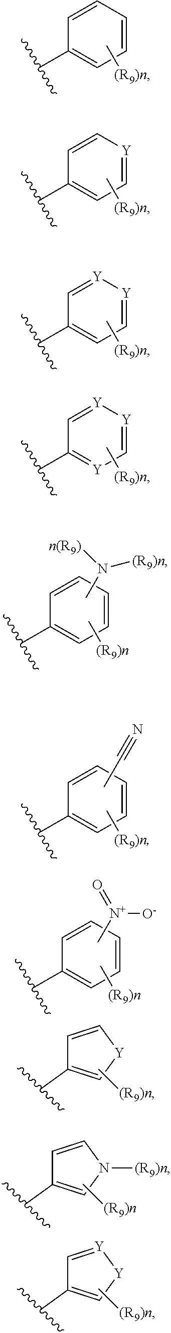 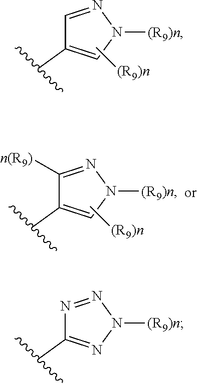
Y is independently O, S, or N;
R₉ is independently H, halogen, C₁₋₆ alkyl, C₁₋₆ alkenyl, C₁₋₆ alkynyl, C₁₋₆ alkoxy, C₀₋₆ amine, C₀₋₆ amide, C₀₋₆—OH, C₀₋₆—COOH, C₀₋₆ CN, C₁₋₆ halogen, or —CF₃; and
n is 0, 1, 2, 3, or 4.

8. The compound of claim 1, wherein each X is S or O and R is:

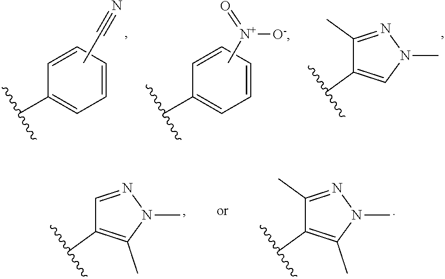

9. The compound of claim 1, wherein each X is S or O and R is:

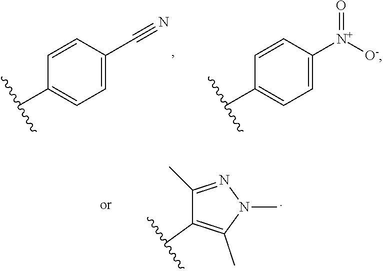

10. The compound of claim 1, wherein the compound is selected from:

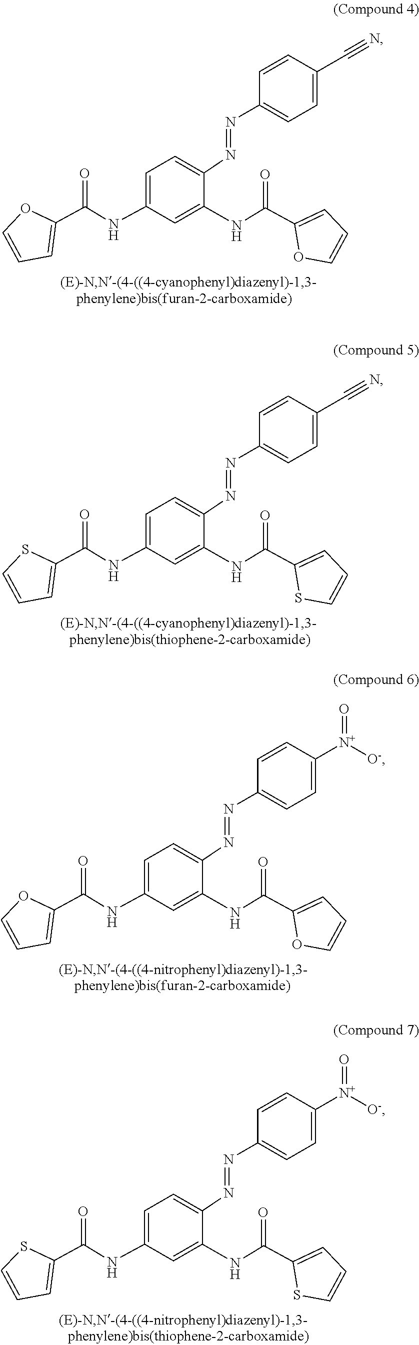

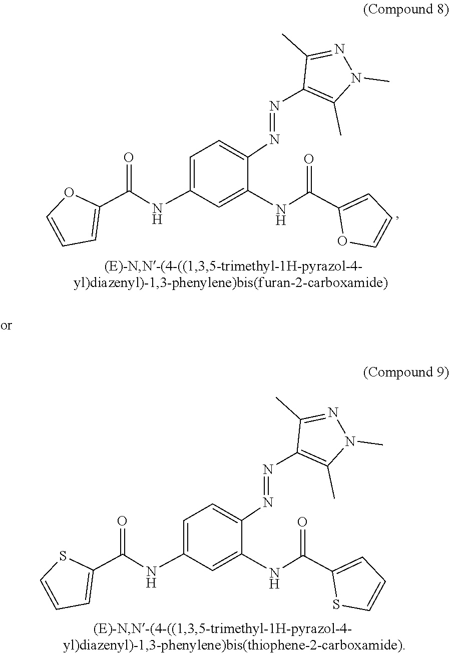 [SMILES: Cc1nn(C)c(C)c1/N=N/c1ccc(NC(=O)c2ccco2)cc1NC(=O)c1ccco1 ; Cc1nn(C)c(C)c1/N=N/c1ccc(NC(=O)c2cccs2)cc1NC(=O)c1cccs1]

11. The compound of claim 1, wherein each X is S or O, and R is a substituted or unsubstituted arylazopyrazole.

12. The compound of claim 1, wherein each X is S or O and R is

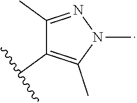

13. The compound of claim 1, wherein the compound is:

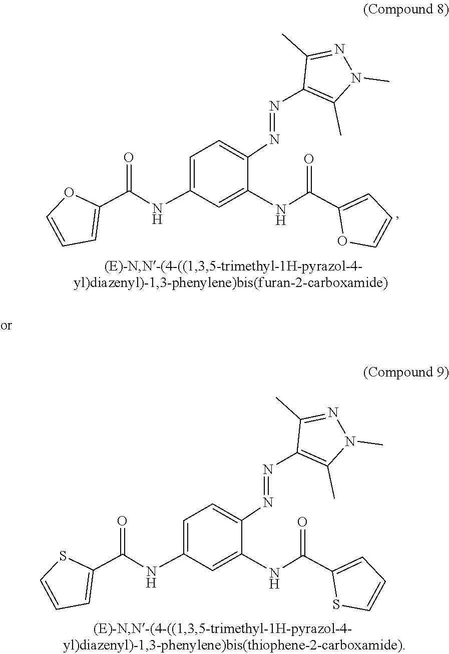 [SMILES: Cc1nn(C)c(C)c1/N=N/c1ccc(NC(=O)c2ccco2)cc1NC(=O)c1ccco1 ; Cc1nn(C)c(C)c1/N=N/c1ccc(NC(=O)c2cccs2)cc1NC(=O)c1cccs1]

14. The compound of claim 1, wherein the compound is:

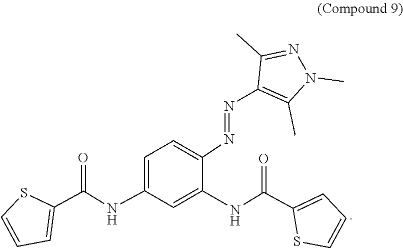 [SMILES: Cc1nn(C)c(C)c1/N=N/c1ccc(NC(=O)c2cccs2)cc1NC(=O)c1cccs1]

15. The compound of claim 1, wherein the compound is a reversible photoswitch that acts on a TRPA1 channel.

16. (canceled)

17. A method for reversibly activating or deactivating a TRPA1 channel, the method comprising:
contacting a TRPA1 channel with an E isomer of a compound of claim 1;
pulse illuminating the compound with violet light (˜350-405 nm) to induce an E→Z isomerization and activate the TRPA1 channel; and
subsequently, pulse illuminating the compound with green light (˜500-600 nm) to induce a Z→E isomerization and deactivate the TRPA1 channel.

18. The method of claim 17, wherein the compound is:

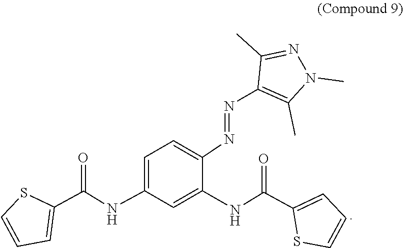 [SMILES: Cc1nn(C)c(C)c1/N=N/c1ccc(NC(=O)c2cccs2)cc1NC(=O)c1cccs1]

19. The method of claim 17, wherein the compound has a concentration of about 10-20 μM.

20. The method of claim 17, wherein the compound is administered to an organism, part thereof, or cell culture, and the organism, part thereof, or cell culture is pulse illuminated with violet light to activate and subsequently green light to deactivate the TRPA1 channel.

21. The method of claim 17, wherein the TRPA1 channel is a Trpa1b channel.

22. The method of claim 17, wherein the TRPA1 channel is a vertebrate Trpa1b channel.

23. The method of claim 17, wherein the TRPA1 channel is a zebrafish (Danio rerio) Trpa1b channel.

24. The method of claim 17, wherein activation of the TRPA1 channel leads to an increase in current and deactivation leads to a decrease in current.

25. (canceled)

26. (canceled)

27. (canceled)

28. (canceled)

29. A kit comprising two or more of: Compound 9, a Trpa1b plasmid (pCMV-zTrpa1b-FLAG; SEQ ID NO:3); Tol2-ngn1-Trpa1b-2A-mCherry (partial vector sequence in SEQ ID NO:5); a zebrafish Trpa1b^{-/-} embryo; a HEK293T cell expressing zebrafish Trpa1b; transfection reagents; buffers and reagents; a light source capable of illuminating in the violet and green wavelengths; packaging, containers, and instructions for use.
